(19) United States
(12) Patent Application Publication
(10) Pub. No.: US 2026/0176280 A1
(43) Pub. Date: Jun. 25, 2026

(54) COMPOUND, COMPOSITION, SURFACE TREATMENT AGENT, ARTICLE, AND METHOD FOR MANUFACTURING ARTICLE

(71) Applicant: AGC Inc., Tokyo (JP)

(72) Inventors: Hironobu SAKAGUCHI, Tokyo (JP); Eiichiro ANRAKU, Tokyo (JP); Shiori KAWAKAMI, Tokyo (JP); Yasuhiro HIRATA, Tokyo (JP)

(73) Assignee: AGC Inc., Tokyo (JP)

(21) Appl. No.: 19/531,873

(22) Filed: Feb. 6, 2026

Related U.S. Application Data
(63) Continuation of application No. PCT/JP2024/028185, filed on Aug. 7, 2024.

(30) Foreign Application Priority Data
Aug. 8, 2023 (JP) 2023-129135

(51) Int. Cl. C07D 487/04; C07D 233/64; C07D 471/04

(52) U.S. Cl. CPC C07D 487/04; C07D 233/64; C07D 471/04

(57) ABSTRACT

A novel compound and the like that are useful as a surface treatment agent capable of forming, on a substrate, a surface treatment layer with excellent water repellency and abrasion resistance are provided. The compound includes heavy hydrogen and the following Group 1, in which a ratio of the heavy hydrogen to light hydrogen is 0.1 mol % or more. Group 1: —Si(R¹)ₙL₃₋ₙ. R¹ is each independently a hydrocarbon group, L is each independently a hydrolyzable group or a hydroxyl group and n is an integer from 0 to 2.

### INCORPORATION BY REFERENCE

This application is based upon and claims the benefit of priority from Japanese patent application No. 2023-129135, filed on Aug. 8, 2023, and PCT application No. PCT/JP2024/028185 filed on Aug. 7, 2024, the disclosure of which is incorporated herein in its entirety by reference.

### BACKGROUND

The present disclosure relates to a compound, a composition, a surface treatment agent, an article, and a method for manufacturing an article.

In recent years, in order to improve performance such as appearance and visibility, a technique for reducing fingerprint adhesion to a surface of an article and a technique for facilitating contaminant removal are desired. As a specific method, a method of performing a surface treatment on a surface of an article using a surface treatment agent is known.

For example, Patent Literature 1 describes a composition comprising an organosilicon compound having at least one trialkylsilyl group and two or more hydrolyzable silyl groups, and a metal compound in which at least one hydrolyzable group is bonded to a metal atom. Patent Literature 2 discloses a specific siloxane group-comprising silane compound including a divalent linear organopolysiloxane group and a hydrolyzable silyl group.
[Patent Literature 1] Japanese Unexamined Patent Application Publication No. 2017-119849
[Patent Literature 2] International Patent Publication No. WO2023/017830

### SUMMARY

Further improvement of surface treatment agents has been required in terms of liquid repellency and the like.

The present disclosure has been made in view of such circumstances, and an object of the present disclosure is to provide a novel compound capable of forming a surface treatment layer with excellent liquid repellency, a composition and a surface treatment agent comprising the novel compound, an article including a surface treatment layer with excellent liquid repellency, and a method for manufacturing the same.

The present disclosure includes the following aspects.

[1]

A compound comprising heavy hydrogen and the following Group 1,
wherein a ratio of the heavy hydrogen to light hydrogen is 0.1 mol % or more

—Si(R¹)ₙL₃₋ₙ Group 1:
where
R¹ is each independently a hydrocarbon group,
L is each independently a hydrolyzable group, a group having a hydrolyzable group, or a hydroxyl group, and
n is an integer from 0 to 2.
   [2]
the heavy hydrogen as a structure of the following Group 2 or Group 3:

—Si(CE_{b1}H_{3-b1})ₐ₁(R²)₂₋ₐ₁—O— Group 2:

-M¹(CE_{b2}H_{3-b2})ₐ₂(R²)₂₋ₐ₂-(Q)ᵣ₁-R¹⁰ Group 3:
where
E is each independently D or T,
R² is each independently a hydrocarbon group,
R¹⁰ is a hydrocarbon group, a trialkylsilyl group, a linear organopolysiloxane group, a cyclic organopolysiloxane group, or a cage-like organopolysiloxane group, and hydrogen atoms in these groups may be heavy hydrogen,
Q is —O— or —OC(═O)—,
M¹ is Si, Sn, or Ge,
r1 is 0 or 1,
a1 is 1 or 2,
b1 is 1, 2, or 3,
a2 is 1 or 2, and
b2 is 1, 2, or 3.
   [3]

The compound according to Item [2], wherein the structure of the Group 2 or Group 3 is represented by the following Group 2a or Group 3a:

—Si(CD₃)₂-O— Group 2a:

-M¹(CD₃)₃ Group 3a:
where M¹ is Si, Sn, or Ge.
   [4]

A compound represented by the following Formula (1a) or the following Formula (2a):

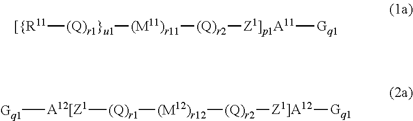

where
R¹¹ is each independently the following Group 3, or a hydrocarbon group, a trialkylsilyl group, a linear organopolysiloxane group, a cyclic organopolysiloxane group, or a cage-like organopolysiloxane group, and hydrogen atoms in these groups may be heavy hydrogen,
M¹¹ is each independently Si, Sn, Ge, or a group including a partial structure having 2 to 5 atoms selected from Si, Sn, and Ge linked together,
M¹² is each independently a group including a partial structure having 2 to 5 atoms selected from Si, Sn, and Ge linked together,
Q is each independently —O— or —OC(═O)—,
u1 is 3 when M¹¹ is Si, Sn, or Ge,
u1 is 1 when M¹¹ includes a partial structure having 2 to 5 atoms selected from Si, Sn, and Ge linked together, or when r11 is 0,
r1 is each independently 0 or 1,
r2 is each independently 0 or 1,
r11 is each independently 0 or 1,
r12 is each independently 0 or 1,
when r11 is 0, r1+r2 is 0 or 1,
when r12 is 0, r1+r2 is 0 or 1,
Z¹ is each independently an alkylene group, a polyalkylene oxide group, an organopolysiloxane group, an organopolysiloxane group including the following Group 2, a combination of an alkylene group with an organopolysiloxane group, or a combination of an alkylene group and an organopolysiloxane group including the following Group 2,
A¹¹ is a single bond or a (p1+q1)-valent linking group,
A¹² is each independently a single bond or a (1+q1)-valent linking group.

G is the following Group 1,
p1 and q1 are each independently an integer of 1 or more,
Group 1 is —Si(R¹)ₙL₃₋ₙ,
Group 2 is —Si(CE_{b1}H_{3-b1})ₐ₁(R²)₂₋ₐ₁—O—,
Group 3 is -M¹(CE_{b2}H_{3-b2})ₐ₂(R²)₂₋ₐ₂-(Q)ᵣ₁-R¹⁰,
R¹ is each independently a hydrocarbon group,
L is each independently a hydrolyzable group, a group having a hydrolyzable group, or a hydroxyl group,
n is an integer from 0 to 2,
E is each independently D or T,
R² is each independently a hydrocarbon group,
R¹⁰ is a hydrocarbon group, a trialkylsilyl group, a linear organopolysiloxane group, a cyclic organopolysiloxane group, or a cage-like organopolysiloxane group, and hydrogen atoms in these groups may be heavy hydrogen,
M¹ is Si, Sn, or Ge,
a1 is 1 or 2,
b1 is 1, 2, or 3,
a2 is 1 or 2, and
b2 is 1, 2, or 3,
where in Formula (1a), at least one of R¹¹ is Group 3, or at least one of R¹¹ and Z¹ includes an organopolysiloxane group including Group 2, and
in Formula (2a), at least one of Z¹ includes an organopolysiloxane group including Group 2.
   [5]

A compound represented by the following Formula (1b) or the following Formula (2b):

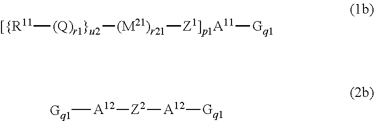

where
R¹¹ is each independently the following Group 3, or a hydrocarbon group, a trialkylsilyl group, a linear organopolysiloxane group, a cyclic organopolysiloxane group, or a cage-like organopolysiloxane group, and hydrogen atoms in these groups may be heavy hydrogen,
Q is each independently —O— or —OC(═O)—,
r1 is each independently 0 or 1,
M²¹ is Si, Sn, or Ge,
r21 is 0 or 1,
u2 is 1 when r21 is 0 and is 3 when r21 is 1,
Z¹ is each independently an alkylene group, a polyalkylene oxide group, an organopolysiloxane group, an organopolysiloxane group including the following Group 2, a combination of an alkylene group with an organopolysiloxane group, or a combination of an alkylene group and an organopolysiloxane group including the following Group 2,
Z² is an organopolysiloxane group including the following Group 2, or a combination of an alkylene group and an organopolysiloxane group including the following Group 2,
A¹¹ is a single bond or a (p1+q1)-valent linking group,
A¹² is each independently a single bond or a (1+q1)-valent linking group, G is the following Group 1,
p1 and q1 are each independently an integer of 1 or more,
Group 1 is —Si(R¹)ₙL₃₋ₙ,
Group 2 is —Si(CE_{b1}H_{3-b1})ₐ₁(R²)₂₋ₐ₁—O—,
Group 3 is -M¹(CE_{b2}H_{3-b2})ₐ₂(R²)₂₋ₐ₂-(Q)ᵣ₁-R¹⁰,
R¹ is each independently a hydrocarbon group,
L is each independently a hydrolyzable group, a group having a hydrolyzable group, or a hydroxyl group,
n is an integer from 0 to 2,
E is each independently D or T,
R² is each independently a hydrocarbon group,
R¹⁰ is a hydrocarbon group, a trialkylsilyl group, a linear organopolysiloxane group, a cyclic organopolysiloxane group, or a cage-like organopolysiloxane group, and hydrogen atoms in these groups may be substituted with the E,
M¹ is Si, Sn, or Ge,
a1 is 1 or 2,
b1 is 1, 2, or 3,
a2 is 1 or 2, and
b2 is 1, 2, or 3,
where in Formula (1b), at least one of R¹¹ is Group 3, or at least one of R¹¹ and Z¹ includes an organopolysiloxane group including Group 2.
   [6]

The compound according to Item [4] or [5], wherein the Z¹ is each independently a group represented by the following Formula (3):

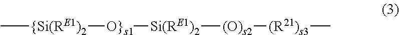

where
R^{E1} is each independently a hydrocarbon group in which a hydrogen atom may be heavy hydrogen,
R²¹ is an alkylene group,
s1 is an integer from 0 to 500,
s2 is 0 or 1, and
s3 is 0 or 1.

The compound according to Item [5], wherein the Z² is a group represented by the following Formula (4):

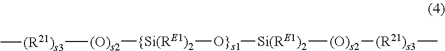

where
R^{E1} is each independently a hydrocarbon group in which a hydrogen atom may be heavy hydrogen,
R²¹ is each independently an alkylene group,
s1 is an integer from 0 to 500,
s2 is each independently 0 or 1, and
s3 is each independently 0 or 1.
   [8]

A compound represented by the following Formula (1c) or the following Formula (2c):

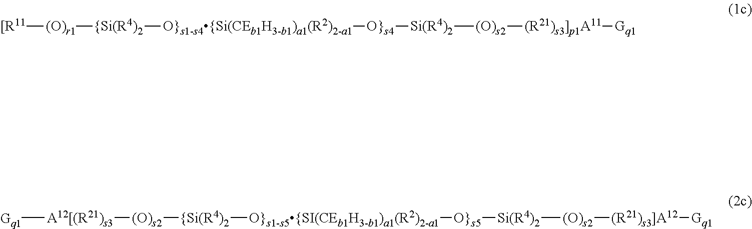

where
R¹¹ is each independently a hydrocarbon group, a trialkylsilyl group, a linear organopolysiloxane group, a cyclic organopolysiloxane group, a cage-like organopolysiloxane group, or the following Group 3,
R⁴ is each independently a hydrocarbon group,
E is each independently D or T,
R²¹ is each independently an alkylene group,
r1 is each independently 0 or 1,
a1 is 1 or 2,
b1 is 1, 2, or 3,
s1 is an integer from 0 to 500,
s2 is each independently 0 or 1,
s3 is each independently 0 or 1,
s4 is an integer from 0 to s1, and is an integer of 1 or more when R¹¹ is a group other than the Group 3,
s5 is an integer from 1 to s1,
A¹¹ is a single bond or a (p1+q1)-valent linking group,
A¹² is each independently a single bond or a (1+q1)-valent linking group.

G is the following Group 1,
p1 and q1 are each independently an integer of 1 or more,
Group 1 is —Si(R¹)ₙL₃₋ₙ,
Group 3 is -M¹(CE_{b2}H_{3-b2})ₐ₂(R²)₂₋ₐ₂-(Q)ᵣ₁-R¹⁰,
R¹ is each independently a hydrocarbon group,
L is each independently a hydrolyzable group, a group having a hydrolyzable group, or a hydroxyl group,
n is an integer from 0 to 2,
E is each independently D or T,
R² is each independently a hydrocarbon group,
R¹⁰ is a hydrocarbon group, a trialkylsilyl group, a linear organopolysiloxane group, a cyclic organopolysiloxane group, or a cage-like organopolysiloxane group, and hydrogen atoms in these groups may be substituted with the E,
M¹ is Si, Sn, or Ge,
a2 is 1 or 2, and
b2 is 1, 2, or 3.
   [9]

The compound according to Item [8], wherein s1 is an integer of 1 or more.

[10]

The compound according to Item[8] or [9], wherein s3 is 1.

[11]

The compound according to any one of Items [8] to [10], wherein the p1 is an integer from 1 to 4.

[12]

The compound according to any one of Items [8] to [11], wherein the q1 is each independently an integer from 1 to 4.

[13]

A composition comprising the compound according to any one of [1] to [12], and a liquid medium.

A surface treatment agent comprising the compound according to any one of [1] to [12].

[15]

A surface treatment agent comprising the compound according to any one of [1] to [12], and a liquid medium.

[16]

A method for manufacturing an article including a surface treatment layer formed on a substrate by performing surface treatment on the substrate by using a surface treatment agent comprising the compound according to any one of [1] to [12].

[17]

An article comprising a substrate, and a surface treatment layer disposed on the substrate, a surface of the surface treatment layer being treated with the surface treatment agent comprising the compound according to any one of [1] to [12].

[18]

The article according to Item [17], wherein the article is an optical member.

[19]

The article according to Item [17] or [18], wherein the article is a display or a touch panel.

According to an embodiment of the present disclosure, it is possible to provide a novel compound capable of forming a surface treatment layer with excellent liquid repellency, a composition and a surface treatment agent comprising the novel compound, an article including a surface treatment layer with excellent liquid repellency, and a method for manufacturing the same.

The above and other objects, features and advantages of the present disclosure will become more fully understood from the detailed description given hereinbelow.

### DESCRIPTION OF EMBODIMENTS

A numerical value range specified by using “to” in the specification of the present disclosure includes numerical values before and after “to” as a lower limit value and an upper limit value, respectively, of the range.

In numerical ranges according to a stepwise manner in the present specification, the upper or lower limit value of one numerical range may be replaced with the upper or lower limit value of another numerical range according to a stepwise manner. Further, in numerical ranges according to the present specification, the upper or lower limit value of a numerical range may be replaced with values shown in Examples.

In the specification of the present disclosure, the “surface treatment layer” refer to a layer that is formed on the surface of a substrate by a surface treatment.

In this specification, when a compound or a group is represented by a specific formula (X), the compound or the group represented by this formula (X) may be expressed as a compound (X) or a compound X, and a group (X) or a group X, respectively.

In this specification, “Heavy Hydrogen”, which is a general term for deuterium (D) and tritium (T), may be abbreviated as (E). “Light Hydrogen” indicates a hydrogen atom (¹H) with one proton in its nucleus.

In this specification, “Me” may refer to a methyl group, “Et” may refer to an ethyl group, and “n-Bu” may refer to an n-butyl group.

When the same symbol exists in a single chemical formula, these symbols may represent structures that are identical to each other, or may represent structures that are different from each other within a defined range.

The bonding direction of a divalent group shown in the specification of the present disclosure is not limited to any particular directions unless otherwise specified. For example, when Y is —COO— in a compound represented by a formula “X—Y—Z”, Y may be —CO—O— or —O—CO—. Further, the aforementioned compound may be “X—CO—O—Z” or “X—O—CO—Z”.

### [Compound]

A compound of the present disclosure includes heavy hydrogen and the following Group 1, and
a ratio of the heavy hydrogen to light hydrogen is 0.1 mol % or more:

—Si(R¹)ₙL₃₋ₙ Group 1:
where
R¹ is each independently a hydrocarbon group,
L is each independently a hydrolyzable group, a group having a hydrolyzable group, or a hydroxyl group, and
n is an integer from 0 to 2.

When a compound of the present disclosure is used as a surface treatment agent, a surface treatment layer with excellent liquid repellency can be formed. Although details of this reason are not clear, it is presumed as follows.

The compound of the present disclosure including the above Group 1 provides a high adhesion between the Group 1 and a substrate, and a surface treatment layer with excellent durability can be formed on the substrate. Further, the compound according to the present disclosure, which contains heavy hydrogen at a ratio exceeding its natural abundance ratio, is estimated to exhibit reduced surface tension due to the heavy hydrogen isotope effect. As a result, a surface treatment layer with excellent liquid repellency (water repellency/oil repellency) can be formed.

### <Group 1>

The compound of the present disclosure includes the above Group 1. In the compound, only one Group 1 may be contained, or two or more Groups 1 may be contained. The number of groups may be, for example, from 1 to 18, from 2 to 12, and from 2 to 8. The Group 1 is a monovalent group, and thus is located at the terminal of the compound.

—Si(R¹)ₙL₃₋ₙ Group 1:
R¹ is each independently a hydrocarbon group, L is each independently a hydrolyzable group, a group having a hydrolyzable group, or a hydroxyl group, and n is an integer from 0 to 2.

In a case where there are a plurality of Groups 1 in one molecule, the plurality of Groups 1 may be the same as each other or different from each other. The plurality of Groups 1 are preferably the same as each other from the viewpoint of availability of raw materials and ease of manufacturing of the compound.

R¹ is a hydrocarbon group. Examples of the hydrocarbon group include an alkyl group, a cycloalkyl group, an alkenyl group, an allyl group, and the like, and the hydrocarbon group is preferably a saturated hydrocarbon group, and more preferably an alkyl group from the viewpoint of ease of synthesis. The number of carbon atoms of R¹ is preferably from 1 to 6, more preferably from 1 to 3, and further preferably from 1 to 2.

L is each independently a hydrolyzable group, a group having a hydrolyzable group, or a hydroxyl group. The hydrolyzable group is a group that becomes a hydroxyl group by a hydrolysis reaction. That is, the hydrolyzable silyl group represented by Si-L undergoes a hydrolysis reaction to become a silanol group represented by Si—OH. The silanol group further reacts between the silanol groups to form a Si—O—Si bond. The silanol group undergoes dehydration condensation reaction with a silanol group derived from an oxide present on the surface of the substrate to allow forming a Si—O—Si bond.

Examples of the hydrolyzable group include an alkoxy group, an aryloxy group, a halogen atom, an acyl group, an acyloxy group, and an isocyanato group (—NCO).

The number of carbon atoms of the alkoxy group is preferably from 1 to 6, and more preferably from 1 to 4. The number of carbon atoms of the aryloxy group is preferably from 3 to 10. The aryl group of the aryloxy group may be a heteroaryl group. The halogen atom is preferably a chlorine atom. The acyl group is preferably an acyl group having a number of carbon atoms from 1 to 6. The acyloxy group is preferably an acyloxy group having a number of carbon atoms from 1 to 6. Further, the alkylene oxide-modified alkoxy group is preferably a group represented by —(O—R⁶¹)ₙ₁₁-L¹ (where R⁶¹ is an alkylene group having a number of carbon atoms from 1 to 10, L¹ is an alkoxy group having a number of carbon atoms from 1 to 6, and n11 is an integer from 1 to 6), and among them, R⁶¹ is preferably an alkylene group having a number of carbon atoms from 1 to 6, and n11 is preferably 1.

Examples of the group having the hydrolyzable group may be the group having the hydrolyzable group illustrated above. The group having the hydrolyzable group is preferably —O-L^{A}-L^{B}. L^{A} is an alkylene group, and L^{B} is a hydrolyzable group. The number of carbon atoms of the alkylene group is preferably from 1 to 10. The hydrolyzable group represented by L^{B} is the same as the hydrolyzable group represented by L described above, and its preferable form is also the same. Specific examples of the group having the hydrolyzable group include —O—CH₂CH₂—OCH₃.

Among them, L is preferably an alkoxy group having a number of carbon atoms from 1 to 4, an alkylene oxide-modified alkoxy group, or a halogen atom from the viewpoint of the ease of manufacturing of the compound. L is preferably an alkoxy group having a number of carbon atoms from 1 to 4, and more preferably an ethoxy group or a methoxy group from the viewpoint of the fact that outgassing during the application is small and more excellent storage stability of the compound.

n is an integer from 0 to 2, preferably 0 or 1, and more preferably 0. The presence of a plurality of L causes stronger adhesion of the surface treatment layer to a substrate.

When n is 1 or smaller, the plurality of L present in one molecule may be the same as each other or different from each other. The plurality of L are preferably the same as each other from the viewpoint of availability of raw materials and ease of manufacturing of the compound. When n is 2, the plurality of R¹ present in one molecule may be the same as each other or different from each other. The plurality of R¹ are preferably the same as each other from the viewpoint of availability of raw materials and ease of manufacturing of the compound.

Specific examples of the Group 1 include —Si(OCH₃)₃, —SiCH₃(OCH₃)₂, —Si(OCH₂CH₃)₃, —SiCl₃, —Si(OCOCH₃)₃, —Si(NCO)₃, and —Si(OCH₂CH₂OCH₃)₃.

### <Heavy Hydrogen>

A compound according to the present disclosure includes heavy hydrogen, and a ratio (E/H) of the heavy hydrogen (E) to light hydrogen (H) is 0.1 mol % or more. By performing surface treatment using the compound of the present disclosure including heavy hydrogen, a surface treatment layer with excellent liquid repellency can be obtained. From the viewpoint of liquid repellency, the E/H is preferably 0.5 mol % or more, and more preferably 0.8 mol % or more. On the other hand, the E/H is normally 50 mol % or less. However, even when the E/H is 20 mol % or less, or 10 mol % or less, it delivers sufficient effects.

Note that the heavy hydrogen rate can be calculated from a result of measuring ²H-NMR of a target compound.

Further, when the position of heavy hydrogen in the compound is specified, a method of Site-specific Natural Isotope Fractionation Nuclear Magnetic Resonance (SNIF-NMR) can be used.

From the viewpoint of forming of a surface treatment layer having more excellent liquid repellency, heavy hydrogen preferably includes a structure of the following Group 2 or Group 3.
Group 2 is —Si(CE_{b1}H_{3-b1})ₐ₁(R²)₂₋ₐ₁—O—
Group 3 is -M¹(CE_{b2}H_{3-b2})ₐ₂(R²)₂₋ₐ₂-(Q)ᵣ₁-R¹⁰
where
E is each independently D or T,
R² is each independently a hydrocarbon group,
R¹⁰ is a hydrocarbon group, a trialkylsilyl group, a linear organopolysiloxane group, a cyclic organopolysiloxane group, or a cage-like organopolysiloxane group, and hydrogen atoms in these groups may be substituted with the E,
Q is —O— or —OC(═O)—,
M¹ is Si, Sn, or Ge,
r1 is 0 or 1,
a1 is 1 or 2,
b1 is 1, 2, or 3,
a2 is 1 or 2, and
b2 is 1, 2, or 3.

### <Group 2>

—Si(CE_{b1}H_{3-b1})ₐ₁(R²)₂₋ₐ₁—O— Group 2:

Only one Group 2 may be contained, or two or more Groups 2 may be contained. When two or more Groups 2 are included, the plurality of Groups 2 may be linked to each other.

R² is a hydrocarbon group. Examples of this hydrocarbon group include an alkyl group, a cycloalkyl group, an alkenyl group, and an allyl group, and the hydrocarbon group is preferably a saturated hydrocarbon group, and more preferably an alkyl group from the viewpoint of ease of synthesis. The number of carbon atoms of R² is preferably from 1 to 6, more preferably from 1 to 3. Among them, —CH₃ or —CH₂CH₃ is preferable.

E is each independently D or T, and is preferably D from the viewpoint of stability of the compound. b1 represents the number of E, and from the viewpoint of liquid repellency, b1 is preferably from 2 to 3, and more preferably 3. Further, a1 represents the number of units of (CE_{b1}H_{3-b1}), and is preferably 2 from the viewpoint of more excellent liquid repellency.

Specific examples of CE_{b1}H_{3-b1} include CD₃, CD₂H, CDH₂, CT₃, CT₂H, CTH₂, CD₂T, CDT₂, and CDTH, preferably CD₃, CD₂H, and CDH₂, and more preferably CD₃.

Specific examples of the Group 2 include —Si(CD₃)₂-O—, —Si(CD₃)(CH₃)—O—, and —Si(CD₃)(CH₂CH₃)—O—.

Further, a bond of Group 2 on the side of Si may be bonded to CE_{b1}H_{3-b1}. In this case, the Group 2 constitutes a terminal represented by (CE_{b1}H_{3-b1})₃Si—O—. Specific examples of the Group 2 that constitutes the terminal include (CD₃)₃Si—O—.

The Group 2 may be contained in an organopolysiloxane group. Examples of the organopolysiloxane group including the Group 2 include a Group (A1).

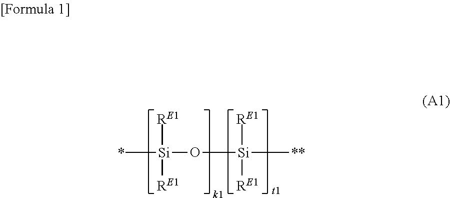

R^{E1} is each independently CE_{b3}H_{3-b3} or R², at least one of R^{E1} is CE_{b3}H_{3-b3}, b3 is 1, 2, or 3, k1 is a number of 1 or more, and t1 is 0 or 1.

R² and E have already been described above. From the viewpoint of liquid repellency, b3 is preferably from 2 to 3, and more preferably 3. k1 may be 1 or more, preferably from 2 to 500, and more preferably from 3 to 300.

Specific examples of the organopolysiloxane group including Group 2 include —Si(CD₃)₂OSi(CD₃)₂O—, and —Si(CD₃)₂OSi(CD₃)₂OSi(CD₃)₂O—.

Group 2 is preferably the following Group 2a from the viewpoint of more excellent liquid repellency.

—Si(CD₃)₂-O— Group 2a:

### <Group 3>

-M¹(CE_{b2}H_{3-b2})ₐ₂(R²)₂₋ₐ₂-(Q)ᵣ₁-R¹⁰ Group 3:

Only one Group 3 may be contained, or two or more Groups 3 may be contained. The Group 3 is a monovalent group, and thus is located at the terminal of the compound.

R² and E are similar to those stated in the above Group 2. b2 is preferably from 2 to 3, and more preferably 3 from the viewpoint of liquid repellency. Further, a2 is preferably 2 from the viewpoint of liquid repellency.

Q is —O— or —OC(═O)—, and when r1 is 0, M¹ and R¹⁰ are bonded directly to each other.

R¹⁰ is a hydrocarbon group, a trialkylsilyl group, a linear organopolysiloxane group, a cyclic organopolysiloxane group, or a cage-like organopolysiloxane group, and hydrogen atoms in these groups may be heavy hydrogen.

Examples of the hydrocarbon group include an alkyl group, a cycloalkyl group, and an aryl group, and the hydrocarbon group is preferably a saturated hydrocarbon group, and more preferably an alkyl group from the viewpoint of ease of synthesis. The number of carbon atoms of R¹⁰ is preferably from 1 to 8, more preferably from 1 to 4, and further preferably from 1 to 2. Specific examples of the hydrocarbon group in R¹⁰ include —CH₃, —CH₂CH₃, —C(CH₃)₃, —CD₃, —CD₂CD₃, and —C(CD₃)₃.

The trialkylsilyl group is preferably a group represented by —SiR⁴⁰₃. R⁴⁰ is each independently a hydrocarbon group. The hydrocarbon group in R⁴⁰ is preferably an alkyl group or an aryl group, and more preferably an alkyl group. The number of carbon atoms of R⁴⁰ is preferably from 1 to 6, more preferably from 1 to 4, and further preferably from 1 to 2. Specific examples of the hydrocarbon group in R⁴⁰ include —CH₃, —CH₂CH₃, —C(CH₃)₃, —CD₃, —CD₂CD₃, and —C(CD₃)₃. When Q is —O— and R¹⁰ is a trialkylsilyl group, QR¹⁰ is a trialkylsilyloxy group.

The linear organopolysiloxane group is preferably the following Group (A2):

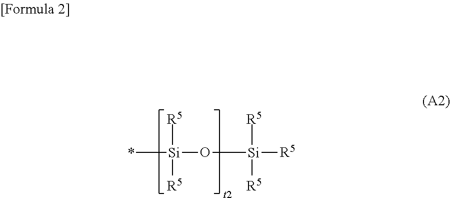

where R⁵ is each independently a hydrocarbon group, and t2 is a number of 1 or more.

The hydrocarbon group in R⁵ is preferably an alkyl group or an aryl group, and more preferably an alkyl group. The number of carbon atoms of R⁵ is preferably from 1 to 6, more preferably from 1 to 4, and further preferably from 1 to 2. Specific examples of the hydrocarbon group in R⁵ include —CH₃, —CH₂CH₃, —C(CH₃)₃, —CD₃, —CD₂CD₃, and —C(CD₃)₃.

k2 may be any number of 1 or more, preferably from 2 to 500, and more preferably from 3 to 300.

The cyclic organopolysiloxane group is preferably the following Group (A3):

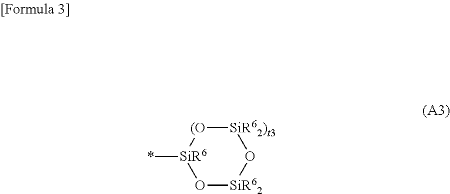

where R⁶ is each independently a hydrocarbon group, a hydrocarbon group having a substituent, or a trialkylsilyloxy group, and t3 is a number from 1 to 4.

The hydrocarbon group in R⁶ is preferably an alkyl group or an aryl group, and more preferably an alkyl group.

The alkyl group is any of a linear alkyl group, a branched alkyl group, and a cyclic alkyl group, and is preferably a linear alkyl group. The number of carbon atoms of the alkyl group is preferably from 1 to 10, more preferably from 1 to 8, and further preferably from 1 to 4. The same holds true for an alkyl group in a trialkylsilyloxy group.

In a hydrocarbon group having a substituent, examples of the substituent include a halogen atom, a hydroxyl group, an alkoxy group, a trialkylsilyloxy group, a trialkylsilyl group, an amino group, a nitro group, a cyano group, a sulfonyl group, and a trifluoromethyl group.

Specific examples of R⁶ include —CH₃, —CH₂CH₃, —C(CH₃)₃, —CD₃, —CD₂CD₃, —C(CD₃)₃, —OSi(CH₃)₃, and —OSi(CD₃)₃.

Specific examples of the cyclic organopolysiloxane group include the following groups. Note that hydrogen may be heavy hydrogen.

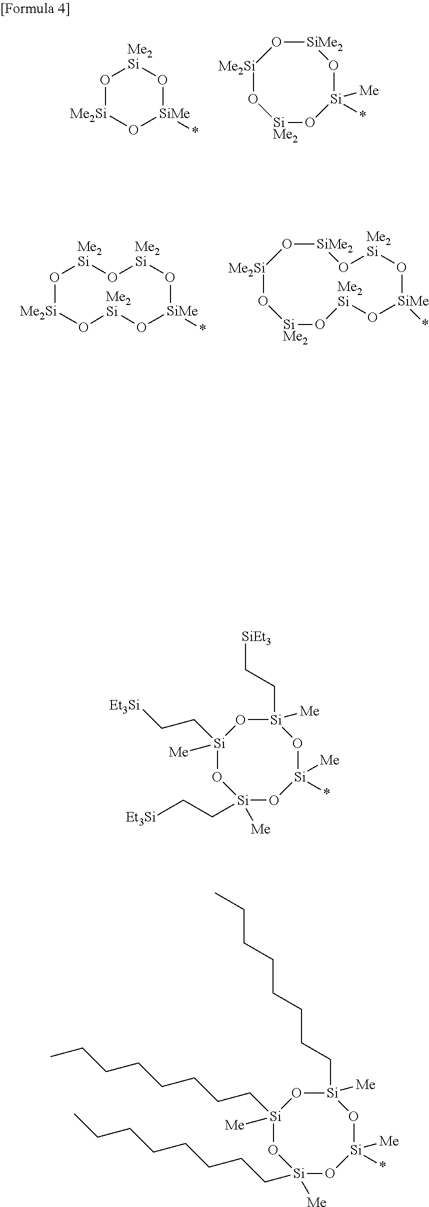

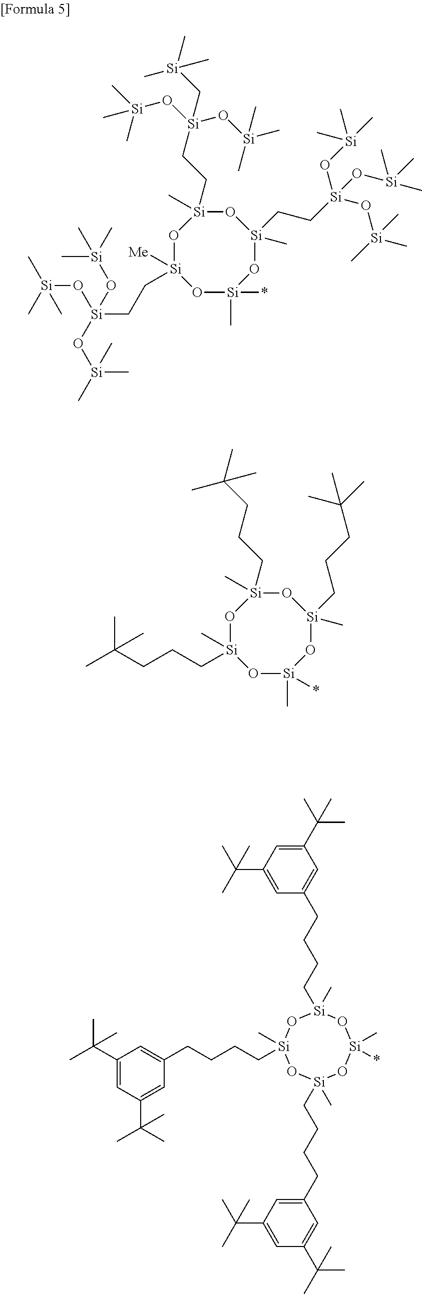

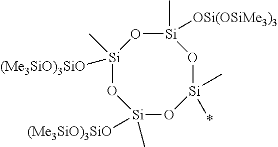

A cage-like organopolysiloxane group is preferably the following Group (A4).

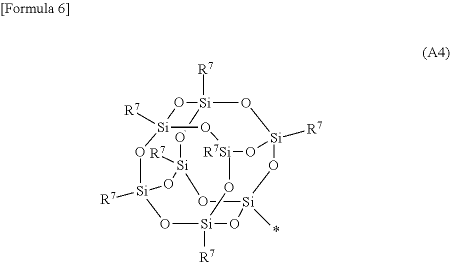

R⁷ is each independently a hydrocarbon group, a hydrocarbon group having a substituent, or a trialkylsilyloxy group.

The hydrocarbon group in R⁷ is preferably an alkyl group or an aryl group, and more preferably an alkyl group.

The alkyl group may be any of a linear alkyl group, a branched alkyl group, and a cyclic alkyl group, and is preferably a linear alkyl group. The number of carbon atoms of the alkyl group is preferably from 1 to 10, more preferably from 1 to 8, and further preferably from 1 to 4. The same holds true for an alkyl group in a trialkylsilyloxy group.

In a hydrocarbon group having a substituent, examples of the substituent include a halogen atom, a hydroxyl group, an alkoxy group, a trialkylsilyloxy group, a trialkylsilyl group, an amino group, a nitro group, a cyano group, a sulfonyl group, and a trifluoromethyl group.

Specific examples of R⁷ include —CH₃, —CH₂CH₃, —C(CH₃)₃, —CD₃, —CD₂CD₃, —C(CD₃)₃, —OSi(CH₃)₃, and —OSi(CD₃)₃.

Specific examples of the cage-like organopolysiloxane group include, for example, the following groups. Note that hydrogen may be heavy hydrogen.

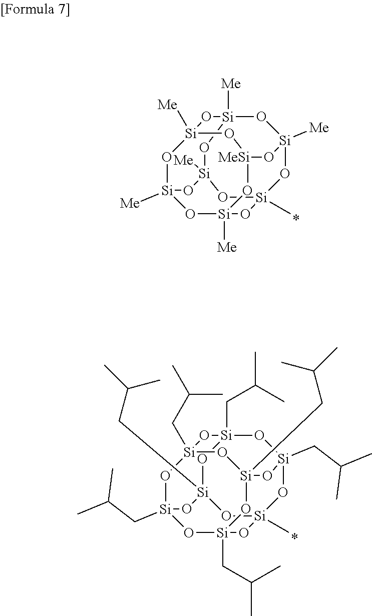

Group 3 is preferably represented by the following Group 3a from the viewpoint of more excellent liquid repellency:

-M¹(CD₃)₃ Group 3a:
where M¹ is Si, Sn, or Ge.

Hereinafter, the compound of the present disclosure will be described more specifically by showing general formulas. However, regarding parts similar to the groups described above, the above descriptions may be referred to and detailed descriptions thereof may be omitted.

<Compound (1a) and Compound (2a)>

One embodiment of the compound according to the present disclosure is a compound represented by the following Formula (1a) or Formula (2a):

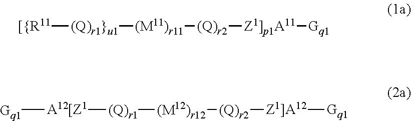

where
R¹¹ is each independently the following Group 3, or is a hydrocarbon group, a trialkylsilyl group, a linear organopolysiloxane group, a cyclic organopolysiloxane group, or a cage-like organopolysiloxane group, and hydrogen atoms in these groups may be heavy hydrogen,
M¹¹ is each independently Si, Sn, Ge, or a group including a partial structure having 2 to 5 atoms selected from Si, Sn, and Ge linked together,
M¹² is each independently a group including a partial structure having 2 to 5 atoms selected from Si, Sn, and Ge linked together,
Q is each independently —O— or —OC(═O)—,
u1 is 3 when M¹¹ is Si, Sn, or Ge,
u1 is 1 when M¹¹ includes a partial structure having 2 to 5 atoms selected from Si, Sn, and Ge linked together, or when r11 is 0,
r1 is each independently 0 or 1,
r2 is each independently 0 or 1,
r11 is each independently 0 or 1,
r12 is each independently 0 or 1,
when r11 is 0, r1+r2 is 0 or 1,
when r12 is 0, r1+r2 is 0 or 1,
Z¹ is each independently an alkylene group, a polyalkylene oxide group, an organopolysiloxane group, an organopolysiloxane group including the following Group 2, a combination of an alkylene group with an organopolysiloxane group, or a combination of an alkylene group and an organopolysiloxane group including the following Group 2,
A¹¹ is a single bond or a (p1+q1)-valent linking group,
A¹² is each independently a single bond or a (1+q1)-valent linking group.

G is the following Group 1,
p1 and q1 are each independently an integer of 1 or more,
Group 1 is —Si(R¹)ₙL₃₋ₙ,
Group 2 is —Si(CE_{b1}H_{3-b1})ₐ₁(R²)₂₋ₐ₁—O—,
Group 3 is -M¹(CE_{b2}H_{3-b2})ₐ₂(R²)₂₋ₐ₂-(Q)ᵣ₁-R¹⁰,
R¹ is each independently a hydrocarbon group,
L is each independently a hydrolyzable group or a hydroxyl group,
n is an integer from 0 to 2,
E is each independently D or T,
R² is each independently a hydrocarbon group,
R¹⁰ is a hydrocarbon group, a trialkylsilyl group, a linear organopolysiloxane group, a cyclic organopolysiloxane group, or a cage-like organopolysiloxane group, and hydrogen atoms in these groups may be heavy hydrogen,
M¹ is Si, Sn, or Ge,
a1 is 1 or 2,
b1 is 1, 2, or 3,
a2 is 1 or 2, and
b2 is 1, 2, or 3,
where in Formula (1a), at least one of R¹¹ is Group 3, or at least one of R¹¹ and Z¹ includes an organopolysiloxane group including Group 2, and
in Formula (2a), at least one of Z¹ includes an organopolysiloxane group including Group 2.

R¹¹ is each independently the following Group 3, or a hydrocarbon group, a trialkylsilyl group, a linear organopolysiloxane group, a cyclic organopolysiloxane group, a cage-like organopolysiloxane group that may contain heavy hydrogen.

Among them, a hydrocarbon group, a trialkylsilyl group, a linear organopolysiloxane group, a cyclic organopolysiloxane group, and a cage-like organopolysiloxane group are the same as those in the R¹⁰. Further, the Group 3 has already been described above.

Q is each independently —O— or —OC(═O)—, and is a single bond in a case where r1 or r2 is 0.

M¹¹ is each independently Si, Sn, Ge, or a group including a partial structure having 2 to 5 atoms selected from Si, Sn, and Ge linked together. A surface treatment layer that uses a compound comprising M¹¹ has excellent liquid repellency.

In a case where M¹¹ is Si, Sn, or Ge, u1 is 3, and the compound (1a) is represented by Formula (1aa):

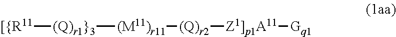

where each of the reference symbols in the Formulas has already been described above.

When M¹¹ is a group including a partial structure having 2 to 5 atoms selected from Si, Sn, and Ge linked together, u1 is 1, and the compound (1a) is represented by the Formula (1ab).

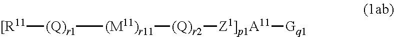

Specific examples of M¹¹ in Formula (1ab) include the following groups. M is each independently Si, Sn, or Ge, R is each independently a hydrocarbon group, and hydrogen may be heavy hydrogen.
-MR₂MR₂—, -MR(MR₃)—,
-MR₂MR₂MR₂—, -MR(MR₃)MR₂—, -MR₂MR(MR₃)—, -M(MR₃)₂—, -MR(MR₂MR₃)—,
-MR₂MR₂MR₂MR₂—, -MR(MR₃)MR₂MR₂—, -MR₂MR(MR₃)MR₂—, -MR₂MR₂MR(MR₃)—, -M(MR₃)₂MR₂—, -MR₂M(MR₃)₂—, -MR(MR₃)MR(MR₃)—, -M(MR₃)(MR₂MR₃)—, -MR(MR₂MR₂MR₃)—,
-MR₂MR₂MR₂MR₂MR₂—, -MR(MR₃)MR₂MR₂MR₂—, -MR₂MR(MR₃)MR₂MR₂—, -MR₂MR₂MR(MR₃)MR₂—, -MR₂MR₂MR₂MR(MR₃)—, -MR(MR₃)MR(MR₃)MR₂—, -MR₂MR(MR₃)MR(MR₃)—, -MR(MR₃)MR₂MR(MR₃)—, -M(MR₃)₂MR₂MR₂—, -MR₂M(MR₃)₂MR₂—, -MR₂MR₂MR(MR₃)₂—, -MR(MR₂MR₃)MR₂MR₂—, -MR₂MR(MR₂MR₃)MR₂—, -MR₂MR₂MR(MR₂MR₃)—, -M(MR₃)(MR₂MR₃)MR₂—, -MR₂M(MR₃)(MR₂MR₃)—, -MR(MR₃)MR(MR₃)₂—, -MR(MR₃)₂MR(MR₃)—, -MR(MR₃)MR(MR₂MR₃)—, -MR(MR₂MR₃)MR(MR₃)—, -MR(MR₂MR₂MR₃)MR₂—, -MR₂MR(MR₂MR₂MR₃)—, -MR(MR₂MR₂MR₂MR₃)—, -MR(MR₃)(MR₂MR₂MR₃)—, -MR(MR₂MR₃)₂—.

While the plurality of units of M in M¹¹ may be the same as each other or different from each other, the plurality of units of M in M¹¹ are preferably the same, and more preferably Si from the viewpoint of the ease of manufacturing. Further, the number of units of M in M¹¹ may be from 2 to 5, and from the viewpoint of the ease of manufacturing, preferably from 2 to 4, and more preferably 2.

Examples of the hydrocarbon group in R include an alkyl group, a cycloalkyl group, and an aryl group, and the hydrocarbon group in R is preferably a saturated hydrocarbon group, and more preferably an alkyl group from the viewpoint of ease of synthesis. The number of carbon atoms of R is preferably from 1 to 8, more preferably from 1 to 4, and further preferably from 1 to 2. Specific examples of the hydrocarbon group in R include —CH₃, —CH₂CH₃, —C(CH₃)₃, —CD₃, —CD₂CD₃, and —C(CD₃)₃.

As M¹¹, —Si(CH₃)₂Si(CH₃)₂—, —Si(CD₃)₂Si(CD₃)₂-, —Si(CH₃)₂Si(CD₃)₂-, —Sn(CH₃)₂Sn(CH₃)₂—, —Sn(CD₃)₂Sn(CD₃)₂-, —Sn(CH₃)₂Sn(CD₃)₂-, —Ge(CH₃)₂Ge(CH₃)₂—, —Ge(CD₃)₂Ge(CD₃)₂-, or —Ge(CH₃)₂Ge(CD₃)₂- is more preferable from the viewpoint of liquid repellency and the ease of manufacturing.

M¹² is each independently a group including a partial structure having 2 to 5 atoms selected from Si, Sn, and Ge linked together. Specific examples and preferable forms thereof are the same as those in the above M¹¹.

r11 is 0 or 1. When r11 is 0, u1 is 1, and r1+r2 is 0 or 1. That is, when r11 is 0, the compound (1a) is represented by Formula (1ac):

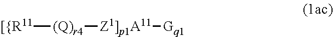

where r4 is 0 or 1, and the other reference symbols are the same as those stated above.

r12 is 0 or 1. When r12 is 0, r1+r2 is 0 or 1. That is, when r12 is 0, the compound (2a) is expressed by Formula (2aa):

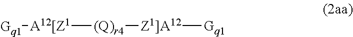

where r4 is 0 or 1, and the other reference symbols are the same as those stated above.

Z¹ is each independently an alkylene group, a polyalkylene oxide group, an organopolysiloxane group, an organopolysiloxane group including Group 2, a combination of an alkylene group with an organopolysiloxane group, or a combination of an alkylene group with an organopolysiloxane group including the Group 2.

The number of carbon atoms of the alkylene group in Z¹ may be 1 or more, and from the viewpoint of water repellency, the number of carbon atoms is preferably 2 or more, and more preferably 4 or more. The upper limit of the number of carbon atoms of the alkylene group is not particularly limited, and is, for example, 30 or less, and preferably 24 or less. The alkylene group in Z¹ may be linear, branched, or cyclic, and is preferably linear or branched, and more preferably linear.

The polyalkylene oxide group is preferably the following Group (A5):

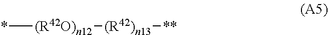

where R⁴² is each independently an alkylene group having a number of carbon atoms from 1 to 10, n12 is an integer from 1 to 200, n13 is 0 or 1, * is a bond on the side of Q, and ** is a bond on the side of A¹¹ or A12.

The number of carbon atoms of the alkylene group in R⁴² is preferably from 1 to 4, and more preferably from 1 to 2 from the viewpoint of the ease of manufacturing. Further, n12 is preferably from 3 to 200, and more preferably from 5 to 150 from the viewpoint of the ease of manufacturing.

The organopolysiloxane group is preferably the following Group (A6).

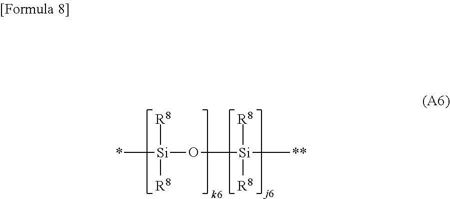

R⁸ is each independently a hydrocarbon group, k6 is an integer of 1 or more, j6 is 0 or 1, * is a bond on the side of Q, and ** is a bond on the side of A11 or A12.

Examples of the hydrocarbon group in R⁸ include an alkyl group, a cycloalkyl group, an alkenyl group, and an allyl group, and the hydrocarbon group in R⁸ is preferably a saturated hydrocarbon group, and more preferably an alkyl group from the viewpoint of ease of synthesis. The number of carbon atoms of R⁸ is preferably from 1 to 6, more preferably from 1 to 3. Among them, —CH₃ or —CH₂CH₃ is preferable.

k6 may be 1 or more, preferably from 2 to 500, and more preferably from 3 to 300.

The organopolysiloxane group including the Group 2 is preferably the Group (A1) according to the above item of <Group 2>.

Specific examples of the combination of the alkylene group with the organopolysiloxane group and the combination of the alkylene group with the organopolysiloxane group including the Group 2 may be the following Groups (B1) to (B4).

*—R^{Si}-Ak¹-** (B1)

*-Ak¹-R^{Si}—** (B2)

*-Ak¹-R^{Si}-Ak¹-** (B3)

*—R^{Si}-Ak¹-R^{Si}—** (B4)

Ak¹ is each independently an alkylene group, R^{Si} is each independently the Group (A1) or the Group (A6), * is a bond on the side of Q, and ** is a bond on the side of A11 or A12.

The number of carbon atoms of the alkylene group in Ak¹ may be 1 or more, preferably 2 or more, and further preferably 4 or more from the viewpoint of water repellency. The upper limit of the number of carbon atoms of the alkylene group is not particularly limited, and is, for example, 30 or less, and preferably 24 or less. The alkylene group in Ak¹ is preferably linear or branched, and more preferably linear.

The combination of the alkylene group with the organopolysiloxane group, or the combination of the alkylene group with the organopolysiloxane group including the Group 2 are preferably the Group (B1), the Group (B3), or the Group (B4), more preferably the Group (B1) or the Group (B4) from the viewpoint of water repellency, and further preferably the Group (B1) from the viewpoint of the ease of manufacturing.

A¹¹ is a single bond or a (p1+q1)-valent linking group. Further, A¹² is a single bond or a (1+q1)-valent linking group. Hereinafter, A¹¹ will be described as a representative example. A¹² is interpreted as the one in which p1 is limited to 1 in the following description of A¹¹.

A¹¹ may be any group that does not impair the effects of the present disclosure, and specific examples of A¹¹ may include an alkylene group that may have an etheric oxygen atom or a divalent organopolysiloxane group, a carbon atom, a nitrogen atom, a silicon atom, an organopolysiloxane group having a valence of 2 to 8, and a group obtained by removing —Si(R¹)ₙL₃₋ₙ (Group 1) from Formulas (3-1A), (3-1B), and (3-1A-1) to (3-1A-7) that will be described later.

In a case where the terminal of Z¹ on the side bonded to A¹¹ is an alkylene group, the terminal of A¹¹ on the side bonded to Z¹ is not an alkylene group. Further, in a case where Z¹ is not a polyalkylene oxide group, the terminal of A¹¹ on the side bonded to Z¹ is not an alkylene oxide group.

Further, A¹¹ may be Groups (g2-1) to (g2-14) that will be described later.

p1 is an integer of 1 or more. From the viewpoint of more excellent water repellency of a surface treatment layer, p1 is preferably from 1 to 6 or from 1 to 4.

q1 is an integer of 1 or more. From the viewpoint of more excellent abrasion resistance of the surface treatment layer, q1 is preferably from 1 to 15, more preferably from 1 to 6, further preferably from 2 to 4, and particularly preferably 2 or 3.

A group represented by A¹¹-G_{q1} in Formula (1a) is preferably a Group (3-1A) or a Group (3-1B), and more preferably a Group (3-1A).

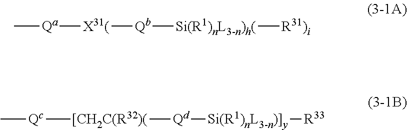

In Formulas (3-1A) and (3-1B), the definitions of R¹, L, and n are the same as those described above.

In Formula (3-1A), Q^{a} is a single bond or a divalent linking group.

Noe that, in a case where the terminal of Z¹ on the side bonded to A¹¹ is an alkylene group, the terminal of Q^{a} on the side bonded to Z¹ is not an alkylene group.

In a case where Z¹ is a polyalkylene oxide group, the terminal of Q^{a} on the side bonded to Z¹ is not an alkylene oxide group.

Specific examples of the divalent linking group include a divalent hydrocarbon group, a divalent heterocyclic group, —O—, —S—, —SO₂—, —N(R^{d})—, —C(O)—, —Si(R^{a})₂—, and a group obtained by combining the two or more kinds of groups of them.

The above divalent hydrocarbon group may be a divalent saturated hydrocarbon group, a divalent aromatic hydrocarbon group, an alkenylene group, or an alkynylene group. The divalent saturated hydrocarbon group may be linear, branched, or cyclic, and examples thereof include an alkylene group. The number of carbon atoms of the alkylene group is preferably from 1 to 30, more preferably from 1 to 20, further preferably from 4 to 20, and particularly preferably from 5 to 15. The divalent aromatic hydrocarbon group is preferably one having a number of carbon atoms from 5 to 20, and examples thereof include a phenylene group. An alkenylene group having a number of carbon atoms from 2 to 20 and an alkynylene group having a number of carbon atoms from 2 to 20 may also be used.

The R^{a} is an alkyl group (preferably having a number of carbon atoms from 1 to 10) or a phenyl group. The R^{d} is a hydrogen atom or an alkyl group (preferably having a number of carbon atoms from 1 to 10).

Examples of the group obtained by combining the two or more kinds of groups of them include —OC(O)—, —C(O)O—, —C(O)S—, —C(O)N(R^{d})—, —N(R^{d})C(O)—, —N(R^{d})C(O)N(R^{d})—, —N(R^{d})C(O)O—, —OC(O)N(R^{d})—, —SO₂N(R^{d})—, —N(R^{d})SO₂—, an alkylene group having —C(O)N(R^{d})—, an alkylene group having —N(R^{d})C(O)—, an alkylene group having —OC(O)N(R^{d})—, an alkylene group having an etheric oxygen atom, an alkylene group having —S—, an alkylene group having —OC(O)—, an alkylene group having —C(O)O—, an alkylene group having —C(O)S—, an alkylene group having —N(R^{d})—, an alkylene group having —N(R^{d})C(O)N(R^{d})—, an alkylene group having —SO₂N(R^{d})—, an alkylene group-Si(R^{a})₂-phenylene group-Si(R^{a})₂.

Among them, in a case where the terminal of Z¹ on the side bonded to A¹¹ is an alkylene group, Q^{a} is preferably a divalent hydrocarbon group other than an alkylene group, a divalent heterocyclic group, —O—, —S—, —SO₂—, —N(R^{d})—, —C(O)—, —Si(R^{a})₂—, —OC(O)—, —C(O)O—, —C(O)S—, —C(O)N(R^{d})—, —N(R^{d})C(O)—, —N(R^{d})C(O)N(R^{d})—, —N(R^{d})C(O)O—, —OC(O)N(R^{d})—, —SO₂N(R^{d})—, —N(R^{d})SO₂—, an alkylene group having —C(O)N(R^{d})—, an alkylene group having —N(R^{d})C(O)—, an alkylene group having —OC(O)N(R^{d})—, an alkylene group having an etheric oxygen atom, an alkylene group having —S—, an alkylene group having —OC(O)—, an alkylene group having —C(O)O—, an alkylene group having —C(O)S—, an alkylene group having —N(R^{d})—, an alkylene group having —N(R^{d})C(O)N(R^{d})—, or an alkylene group having —SO₂N(R^{d})—, more preferably —OC(O)—, an alkylene group having —C(O)N(R^{d})—, an alkylene group having —OC(O)N(R^{d})—, an alkylene group having an etheric oxygen atom, an alkylene group having —S—, an alkylene group having —C(O)O—, an alkylene group having —C(O)S—, an alkylene group having —N(R^{d})—, or an alkylene group having —N(R^{d})C(O)N(R^{d})—, and further preferably an alkylene group having —C(O)O—, or an alkylene group having —C(O)N(R^{d})—.

In a case where Z¹ is a polyalkylene oxide group, Q^{a} is preferably a divalent hydrocarbon group, a divalent heterocyclic group, —SO₂—, —C(O)—, —Si(R^{a})₂—, —C(O)O—, an —C(O)N(R^{d})—, —SO₂N(R^{d})—, an alkylene group having —C(O)N(R^{d})—, an alkylene group having —C(O)O—, an alkylene group having —SO₂N(R^{d})—, an alkylene group-Si(R^{a})₂-phenylene group-Si(R^{a})₂, and more preferably —C(O)—.

In Formula (3-1A), X³¹ is a single bond, an alkylene group, a carbon atom, a nitrogen atom, a silicon atom, an organopolysiloxane group having a valence of 2 to 8, or a group having a (h+i+1)-valent ring.

In a case where the terminal of Z¹ on the side bonded to A¹¹ is an alkylene group and Q^{a} is a single bond, the terminal of X³¹ on the side bonded to Z¹ is not an alkylene group.

In addition, in a case where Z¹ is a polyalkylene oxide group and Q^{a} is a single bond, the terminal of X³¹ on the side bonded to Z¹ is not a polyalkylene oxide group. In a case other than the above, the terminal of X³¹ may be an alkylene group or an alkylene oxide group.

The above alkylene group may have —O—, a silphenylene skeleton group, a divalent organopolysiloxane group, or a dialkylsilylene group. The alkylene group may have a plurality of groups selected from the group consisting of —O—, a silphenylene skeleton group, a divalent organopolysiloxane group, and a dialkylsilylene group.

The number of carbon atoms of the alkylene group represented by X³¹ is preferably from 1 to 20, and more preferably from 1 to 10.

Examples of the organopolysiloxane group having a valence of 2 to 8 include a divalent organopolysiloxane group, and a (w+1)-valent organopolysiloxane group described later.

In Formula (3-1A), when X³¹ is a group having a (h+i+1)-valent ring, Q^{a}, (-Q^{b}-Si¹(R)ₙL₃₋ₙ), and R³¹ are directly bonded to atoms constituting this ring. However, this ring is a ring other than the organopolysiloxane ring.

The ring in X³¹ may be any of a monocyclic ring, a condensed polycyclic ring, a bridged ring, a spiro ring, and an assembled polycyclic ring, and the atoms constituting the ring may be a carbon ring consisting solely of carbon atoms or a heterocyclic ring consisting of heteroatoms having a valence of 2 or more and carbon atoms. Further, the bond between the atoms constituting the ring may be a single bond or multiple bonds. Further, the ring may be an aromatic ring or a non-aromatic ring.

The monocyclic ring is preferably a 4 to 8 membered ring, and more preferably a 5 membered ring and a 6 membered ring. The condensed polycyclic ring is preferably a condensed polycyclic ring in which two or more 4 to 8 membered rings are condensed, more preferably a condensed polycyclic ring in which two or three rings each selected from a 5 membered ring and a 6 membered ring are bonded, and still more preferably a condensed polycyclic ring in which one or two rings each selected from a 5 membered ring and a 6 membered ring and one 4 membered ring are bonded. The bridged ring is preferably a bridged ring in which a 5 membered ring or a 6 membered ring is the largest ring, and the spiro ring is preferably a spiro ring consisting of two 4 to 6 membered rings. The assembled polycyclic ring is preferably an assembled polycyclic ring in which two or three rings each selected from a 5 membered ring and a 6 membered ring are bonded through a single bond, through one to three carbon atoms, or through one heteroatom having a valence of two or three. Note that in the assembled polycyclic ring, one of Q^{a}, (-Q^{b}-Si(R¹)ₙL₃₋ₙ), and R³¹ (when i=1 or more) is preferably bonded to each ring.

The heteroatoms constituting the aforementioned ring are preferably nitrogen atoms, oxygen atoms, and sulfur atoms, and more preferably nitrogen atoms and oxygen atoms. The number of heteroatoms constituting the ring is preferably three or smaller. Further, when the number of heteroatoms constituting the ring is two or more, these heteroatoms may be different from one another.

The ring in X³¹ is preferably one ring selected from the group consisting of a 3 to 8 membered aliphatic ring, a benzene ring, a 3 to 8 membered heterocyclic ring, a condensed ring in which two or three of these rings are condensed, a bridged ring in which a 5 membered ring or a 6 membered ring is the largest ring, and an assembled polycyclic ring having two or more of these rings in which an alkylene group having a number of carbon atoms of 3 or smaller of which the linking group is a single bond, an oxygen atom, or a sulfur atom in view of the ease of manufacturing of the compound and because the abrasion resistance of the surface treatment layer becomes more excellent.

Preferred rings are a benzene ring, a 5 or 6 membered aliphatic ring, a 5 or 6 membered heterocyclic ring having a nitrogen atom or an oxygen atom, and a condensed ring consisting of a 5 or 6 membered carbon ring and a 4 to 6 membered heterocyclic ring.

Specific examples of the ring include the below-shown rings, a 1,3-cyclohexadiene ring, a 1,4-cyclohexadiene ring, an anthracene ring, a cyclopropane ring, a decahydronaphthalene ring, a norbornene ring, a norbornadiene ring, a furan ring, a pyrrole ring, a thiophene ring, a pyrazine ring, a morpholine ring, an aziridine ring, an isoquinoline ring, an oxazole ring, an isoxazole ring, a thiazole ring, an imidazole ring, a pyrazole ring, a pyran ring, a pyridazine ring, a pyrimidine ring, and an indene ring. Note that rings having an oxo Group (═O) are also shown below.

### [Formula 9]

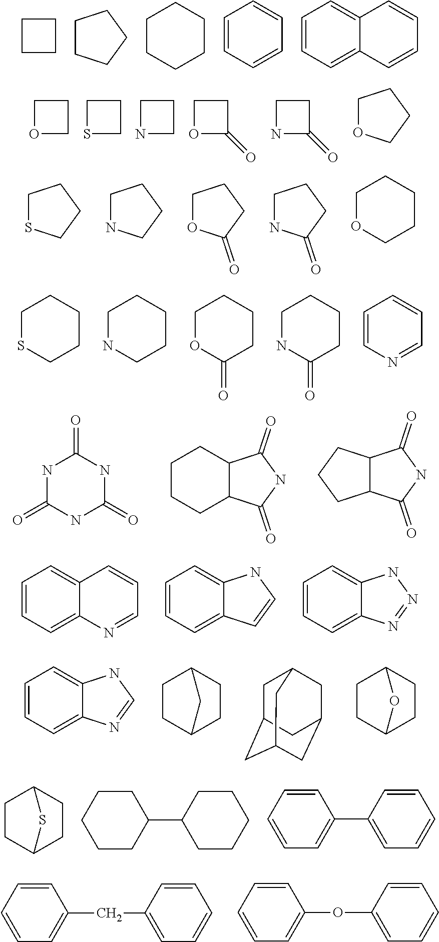

A bond that does not constitute a ring of atoms constituting the ring in X³¹ is a bond bonded to Q^{a}, (-Q^{b}-Si(R¹)ₙL₃₋ₙ), or R³¹. When there are remaining bonds, the remaining bonds are bonded to a hydrogen atom or a substituent. Specific examples of the substituent include a halogen atom, an alkyl group (an etheric oxygen atom may be contained between carbon atoms), a cycloalkyl group, an alkenyl group, an allyl group, an alkoxy group, and an oxo group (═O).

Further, in a case where one of the carbon atoms constituting the ring has two bonds bonded to Q^{a}, (-Q^{b}-Si(R¹)ₙL₃₋ₙ), or R³¹, Q^{a} and (-Q^{b}-Si(R¹)ₙL₃₋ₙ) may be bonded to one of the carbon atoms, or two (-Q^{b}-Si(R²)ₙL₃₋ₙ) may be bonded to one of the carbon atoms. Q^{a}, and (-Q^{b}-Si(R¹)ₙL₃₋ₙ) or R³¹ are preferably bonded to another ring-constituting atom. Each of h units of (-Q^{b}-Si(R¹)ₙL₃₋ₙ) may be bonded to a separate ring-constituting atom, and two of them may be bonded to one ring-constituting carbon atom, and there may be two or more ring-constituting carbon atoms to which two units of (-Q^{b}-Si(R¹)ₙL₃₋ₙ) are bonded. Each of i units of R³¹ may be bonded to a separate ring-constituting atom, two units of them may be bonded to one ring-constituting carbon atom, and further, there may be two or more ring-constituting carbon atoms to which two units of R³¹ are bonded.

Among them, X³¹ is preferably a carbon atom, a nitrogen atom, a silicon atom, an organopolysiloxane group having a valence of 4 to 8, or a group having a (h+i+1)-valent ring, and more preferably a carbon atom from the viewpoint of improving abrasion resistance of the surface treatment layer.

In Formula (3-1A), Q^{b} is a single bond or a divalent linking group.

The definition of the divalent linking group is the same as the definition according to Q^{a} described above.

In a case where the terminal of Z¹ on the side bonded to A¹¹ is an alkylene group and Q^{a} and X³¹ have a single bond, the terminal of Q^{b} on the side bonded to Z¹ is not an alkylene group.

Further, in a case where Z¹ is a polyalkylene oxide group and Q^{a} and X³¹ have a single bond, the terminal of Q^{b} on the side bonded to Z is not an alkylene oxide group. In a case other than the above, the terminal of Q^{b} may be an alkylene group or an alkylene oxide group.

Among them, Q^{b} is preferably an alkylene group which may have an etheric oxygen atom. The number of carbon atoms of the alkylene group is preferably from 1 to 30, more preferably from 1 to 20, further preferably from 2 to 20, and may be from 2 to 10, may be from 2 to 6, or may be from 2 to 5. The number of carbon atoms may be, for example, 2, 3, 8, 9, or 11. Further, the number of carbon atoms may be from 1 to 10.

In Formula (3-1A), R³¹ is a hydrogen atom, a hydroxyl group, or an alkyl group.

The number of carbon atoms of the alkyl group is preferably from 1 to 5, more preferably from 1 to 3, and further preferably 1.

When X³¹ is a single bond or an alkylene group, h is 1 and i is 0,
when X³¹ is a nitrogen atom, h is an integer from 1 to 2, i is an integer from 0 to 1, and h+i=2 is satisfied,
when X³¹ is a carbon atom or a silicon atom, h is an integer from 1 to 3, i is an integer from 0 to 2, and h+i=3 is satisfied,
when X³¹ is an organopolysiloxane group having a valence of 2 to 8, h is an integer from 1 to 7, i is an integer from 0 to 6, and h+i=1 to 7 is satisfied.

When X³¹ is a group having a (h+i+1)-valent ring, h is an integer from 1 to 7, i is an integer from 0 to 6, and h+i=1 to 7 is satisfied.

In a case where there are two or more units of (-Q^{b}-Si(R¹)ₙL₃₋ₙ), two or more units of (-Q^{b}-Si(R¹)ₙL₃₋ₙ) may be the same as each other or different from each other. In a case where there are two or more units of R³¹, two or more units of (—R³¹) may be the same as each other or different from each other.

Among them, i is preferably 0 from the viewpoint of improving abrasion resistance of the surface treatment layer.

In Formula (3-1A), in a case where Q^{a}, X³¹, and Q^{b} have a single bond, [Si(R¹)ₙL₃₋ₙ] is directly bonded to Z¹, and Z¹ is an alkylene group.

In Formula (3-1B), Q^{c} is a single bond or a divalent linking group.

However, in a case where the terminal of Z¹ on the side bonded to A¹¹ is an alkylene group, the terminal of Q^{c} on the side bonded to Z¹ is not an alkylene group.

In a case where Z¹ is a polyalkylene oxide group, the terminal of Q^{c} on the side bonded to Z¹ is not an alkylene oxide group.

The definition of the divalent linking group is the same as the definition according to Q^{a} described above.

In Formula (3-1B), R³² is a hydrogen atom or an alkyl group having a number of carbon atoms from 1 to 10, and is preferably a hydrogen atom from the viewpoint of the ease of manufacturing of the compound.

The alkyl group is preferably a methyl group.

In Formula (3-1B), Q^{d} is a single bond or an alkylene group. The number of carbon atoms of the alkylene group is preferably from 1 to 10, and more preferably from 1 to 6. Q^{d} is preferably a single bond or CH₂— from the viewpoint of the ease of manufacturing of the compound.

In Formula (3-1B), R³³ is a hydrogen atom or a halogen atom, and is preferably a hydrogen atom from the viewpoint of the ease of manufacturing of the compound.

y is an integer from 1 to 10, and preferably an integer from 1 to 6.

The two or more units of [CH₂C(R³²)(-Q^{d}-Si(R¹)ₙL₃₋ₙ)] may be the same as each other or different from each other.

The Group (3-1A) is preferably one of Groups (3-1A-1) to (3-1A-7).

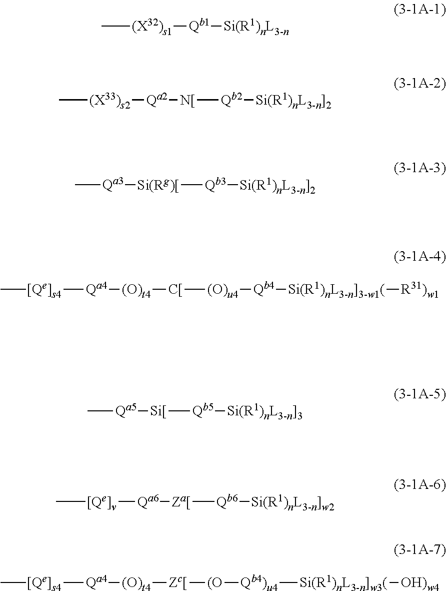

Note that in Formulas (3-1A-1) to (3-1A-7), the definitions of R¹, L, and n are the same as those described above.

Note that, in a case where the terminal of Z¹ on the side bonded to A¹¹ is an alkylene group, the terminal of the Groups (3-1A-1) to (3-1A-7) on the side bonded to Z¹ is not an alkylene group.

In a case where Z¹ is a polyalkylene oxide group, the terminal of the Groups (3-1A-1) to (3-1A-7) on the side bonded to Z¹ is not an alkylene oxide group.

Among them, the Group (3-1A) is preferably a Group (3-1A-4).

In the Group (3-1A-1), X³² is —O—, —S—, —N(R^{d})—, —C(O)—, —C(O)O—, —C(O)S—, —SO₂N(R^{d})—, —N(R^{d})SO₂—, —N(R^{d})C(O)—, —N(R^{d})C(O)N(R^{d})—, —OC(O)N(R^{d})—, or —C(O)N(R^{d})— (note that N in the formula is bonded to Q^{b1}).

The definition of R^{d} is the same as that described above.

s1 is 0 or 1.

Among them, in a case where the terminal of Z¹ on the side bonded to A¹¹ is an alkylene group, X³² is preferably —O—, —S—, —N(R^{d})—, —C(O)—, —C(O)O—, —C(O)S—, —SO₂N(R^{d})—, —N(R^{d})SO₂—, —N(R^{d})C(O)—, —N(R^{d})C(O)N(R^{d})—, —OC(O)N(R^{d})—, or —C(O)N(R^{d})—, more preferably —O—, —S—, —N(R^{d})—, —C(O)O—, —C(O)S—, —N(R^{d})C(O)N(R^{d})—, —OC(O)N(R^{d})—, or —C(O)N(R^{d})—, and further preferably —C(O)O— or —C(O)N(R^{d})—. In a case where Z¹ is a polyalkylene oxide group, X³² is preferably —C(O)O—, —C(O)S—, —SO₂N(R^{d})—, or —C(O)N(R^{d})—, and more preferably —C(O)—.

Q^{b1} is a single bond or an alkylene group. The alkylene group may have —O—, a silphenylene skeleton group, or a dialkylsilylene group. The alkylene group may have a plurality of groups selected from the group consisting of —O—, a silphenylene skeleton group, a divalent organopolysiloxane group, and a dialkylsilylene group.

When the alkylene group has —O—, a silphenylene skeleton group, a divalent organopolysiloxane group, or a dialkylsilylene group, it is preferable to have these groups between carbon atoms.

The number of carbon atoms of the alkylene group represented by Q^{b1} is preferably from 1 to 30, more preferably from 1 to 20, further preferably from 2 to 20, and particularly preferably from 2 to 6. Further, the number of carbon atoms may be from 1 to 10.

Among them, in a case where the terminal of Z¹ on the side bonded to A¹¹ is an alkylene group, s1 is preferably 1, and Q^{b1} is preferably an alkylene group having a number of carbon atoms from 2 to 6.

In a case where Z¹ is a polyalkylene oxide group, s1 is preferably 0, and Q^{b1} is preferably an alkylene group having a number of carbon atoms from 2 to 6.

In a case where the terminal of Z¹ on the side bonded to A¹¹ is an alkylene group, specific examples of the Group (3-1A-1) include the following groups. In the following formula, * represents a bonding position with Z¹.

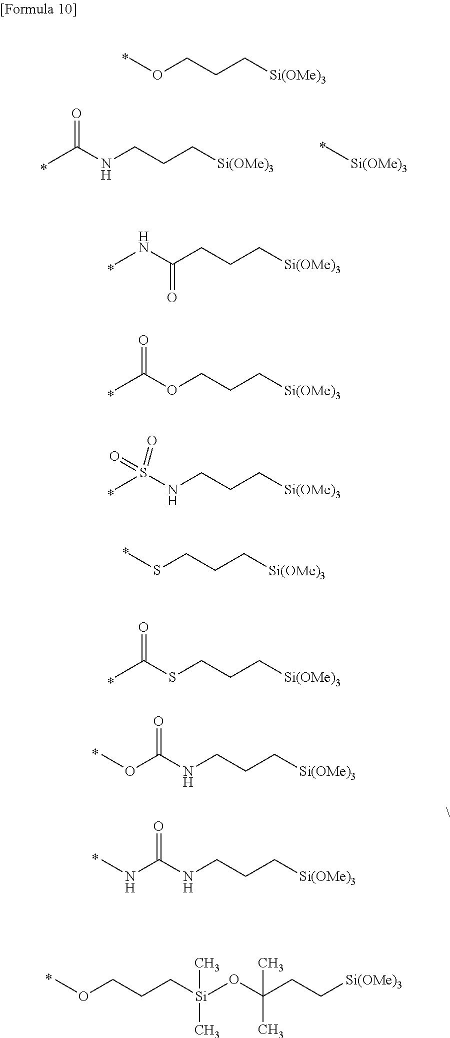

In a case where Z¹ is a polyalkylene oxide group, specific examples of the Group (3-1A-1) include the following groups. In the following formula, * represents a bonding position with Z¹.

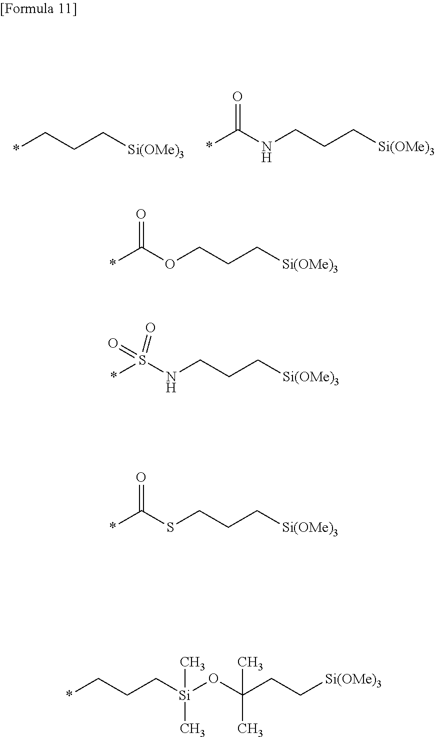

In the Group (3-1A-2), X³³ is —O—, —S—, —N(R^{d})—, —C(O)—, —C(O)O——C(O)S—, —SO₂N(R^{d})—, —N(R^{d})SO₂—, —N(R^{d})C(O)—, —N(R^{d})C(O)N(R^{d})—, —OC(O)N(R^{d})—, or —C(O)N(R^{d})—.

The definition of R^{d} is the same as that described above.

s2 is 0 or 1. s2 is preferably 0 from the viewpoint of the ease of manufacturing of the compound.

Among them, in a case where the terminal of Z¹ on the side bonded to A¹¹ is an alkylene group, X³³ is preferably —O—, —S—, —N(R^{d})—, —C(O)—, —C(O)O—, —C(O)S—, —SO₂N(R^{d})—, —N(R^{d})SO₂—, —N(R^{d})C(O)—, —N(R^{d})C(O)N(R^{d})—, —OC(O)N(R^{d})—, or —C(O)N(R^{d})—.

When Z¹ is a polyalkylene oxide group, X³³ is preferably —C(O)O—, —C(O)S—, —SO₂N(R^{d})—, or —C(O)N(R^{d})—.

Q^{a2} is a single bond, an alkylene group, —C(O)—, or a group having an etheric oxygen atom, —C(O)—, —C(O)O—, —OC(O)—, —C(O)N(R^{d})—, —N(R^{d})C(O)—, —N(R^{d})C(O)N(R^{d})—, —N(R^{d})C(O)O—, —OC(O)N(R^{d})—, —SO₂N(R^{d})—, —N(R^{d})SO₂—, —C(O)N(R^{d})—, or —NH— between carbon atoms of an alkylene group having a number of carbon atoms of 2 or more.

The number of carbon atoms of the alkylene group represented by Q^{a2} is preferably from 1 to 20, more preferably from 1 to 10, further preferably from 1 to 6, and particularly preferably from 1 to 3.

The number of carbon atoms of the group having an etheric oxygen atom, —C(O)—, —C(O)O—, —OC(O)—, —C(O)N(R^{d})—, —N(R^{d})C(O)—, —N(R^{d})C(O)N(R^{d})—, —N(R^{d})C(O)O—, —OC(O)N(R^{d})—, —SO₂N(R^{d})—, —N(R^{d})SO₂—, —C(O)N(R^{d})—, or —NH— between carbon atoms of an alkylene group having a number of carbon atoms of 2 or more represented by Q^{a2} is preferably from 2 to 10, and more preferably from 2 to 6.

Q^{a2} is preferably a single bond from the viewpoint of the ease of manufacturing of the compound.

Q^{b2} is an alkylene group, or a group having a divalent organopolysiloxane group, an etheric oxygen atom, or —NH— between carbon atoms of an alkylene group having a number of carbon atoms of 2 or more.

The number of carbon atoms of the alkylene group represented by Q^{b2} is preferably from 1 to 30, more preferably from 1 to 20, further preferably from 2 to 20, may be from 2 to 10, and may be from 2 to 6. The number of carbon atoms may be, for example, 2, 3, 8, 9, or 11. Further, the number of carbon atoms may be from 1 to 10.

The number of carbon atoms of the group having a divalent organopolysiloxane group or a group having an etheric oxygen atom or —NH— between carbon atoms of the alkylene group having a number of carbon atoms of 2 or more represented by Q^{b2} is preferably from 2 to 10, and more preferably from 2 to 6.

Q^{b2} is preferably —CH₂CH₂CH₂— or —CH₂CH₂OCH₂CH₂CH₂— from the viewpoint of the ease of manufacturing of the compound (note that the right side is bonded to Si).

Two units of [-Q^{b2}-Si(R¹)ₙL₃₋ₙ] may be the same as each other or different from each other.

In a case where the terminal of Z¹ on the side bonded to A¹¹ is an alkylene group, specific examples of the Group (3-1A-2) include the following groups. In the following formula, * represents a bonding position with Z¹. Further, in the formula, α in (CH₂)_{α} that is bonded to a reactive silyl group is an integer indicating the number of methylene groups, is preferably from 1 to 30, more preferably from 1 to 20, further preferably from 2 to 20, may be from 2 to 10, and may be from 2 to 6. The number of carbon atoms may be, for example, 2, 3, 8, 9, or 11. Further, the number of carbon atoms may be from 1 to 10. A plurality of a contained in the same compound may be the same as each other or different from each other, and are preferably the same as each other. For example, the plurality of α contained in the same compound are all 2, 3, 8, 9, and 11. The same applies hereinafter.

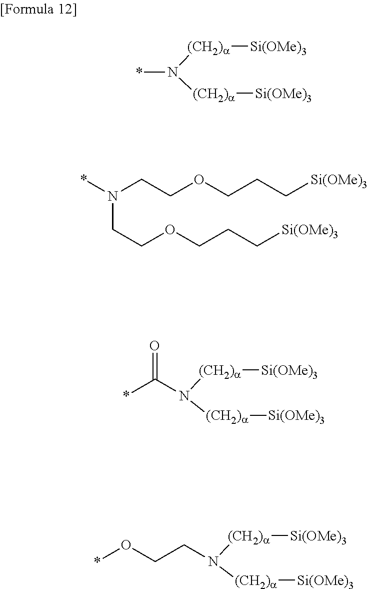

In a case where Z¹ is a polyalkylene oxide group, specific examples of the Group (3-1A-2) include the following groups. In the following formula, * represents a bonding position with Z¹.

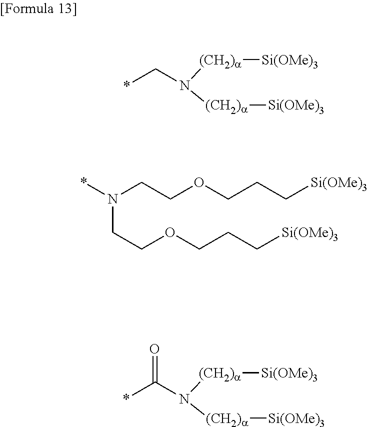

In the Group (3-1A-3), Q^{a3} is a single bond or an alkylene group which may have an etheric oxygen atom. From the viewpoint of the ease of manufacturing of the compound, Q^{a3} is preferably a single bond.

The number of carbon atoms of the alkylene group which may have an etheric oxygen atom is preferably from 1 to 10, and particularly preferably from 2 to 6.

R⁹ is a hydrogen atom, a hydroxyl group, or an alkyl group.

R⁹ is preferably a hydrogen atom or an alkyl group from the viewpoint of the ease of manufacturing of the compound. The number of carbon atoms of the alkyl group is preferably from 1 to 10, more preferably from 1 to 4, and further preferably a methyl group.

Q^{b3} is an alkylene group, or a group having an etheric oxygen atom or a divalent organopolysiloxane group between carbon atoms of the alkylene group having a number of carbon atoms of 2 or more.

The number of carbon atoms of the alkylene group represented by Q^{b3} is preferably from 1 to 30, more preferably from 1 to 20, further preferably from 2 to 20, may be from 2 to 10, and may be from 2 to 6. The number of carbon atoms may be, for example, 2, 3, 8, 9, or 11. Further, the number of carbon atoms may be from 1 to 10.

The number of carbon atoms of the group having an etheric oxygen atom or a divalent organopolysiloxane group between carbon atoms of the alkylene group having a number of carbon atoms of 2 or more represented by Q^{b3} is preferably from 2 to 20, more preferably from 2 to 10, and further preferably from 2 to 6.

Q^{b3} is preferably —CH₂CH₂—, —CH₂CH₂CH₂—, or —CH₂CH₂CH₂CH₂CH₂CH₂CH₂CH₂— from the viewpoint of the ease of manufacturing of the compound.

Two units of [-Q^{b3}-Si(R¹)ₙL₃₋ₙ] may be the same as each other or different from each other.

In a case where the terminal of Z¹ on the side bonded to A¹¹ is an alkylene group, specific examples of the Group (3-1A-3) include the following groups. In the following formula, * represents a bonding position with Z¹.

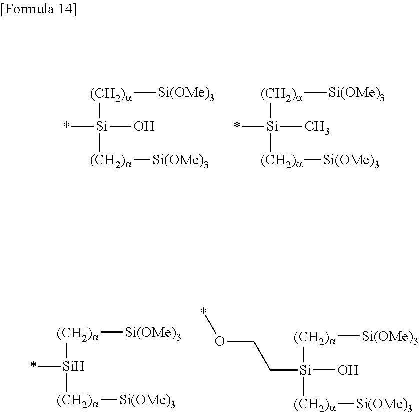

In a case where Z¹ is a polyalkylene oxide group, specific examples of the Group (3-1A-3) include the following groups. In the following formula, * represents a bonding position with Z¹.

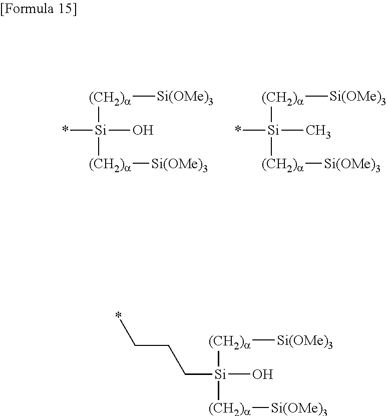

In the Group (3-1A-4), Q^{e} is —C(O)O—, —SO₂N(R^{d})—, —N(R^{d})SO₂—, —N(R^{d})C(O)—, or —C(O)N(R^{d})—.

The definition of R³¹ is the same as that described above. When w1 is 1 or 2, R³¹ is preferably a hydrogen atom.

s4 is 0 or 1.

Q^{a4} is a single bond, or an alkylene group which may have an etheric oxygen atom.

The number of carbon atoms of the alkylene group which may have an etheric oxygen atom is preferably from 1 to 20, more preferably from 1 to 10, further preferably from 1 to 6, and particularly preferably from 1 to 3.

t4 is 0 or 1 (when Q^{a4} is a single bond, t4 is 0).

As -Q^{a4}-(O)ₜ₄—, in a case where s4 is 0, a single bond, —CH₂O—, —CH₂OCH₂—, —CH₂OCH₂CH₂O—, —CH₂OCH₂CH₂OCH₂—, or —CH₂OCH₂CH₂CH₂CH₂OCH₂— is preferable, and in a case where s4 is 1, a single bond, —CH₂—, or —CH₂CH₂— is preferable from the viewpoint of the ease of manufacturing of the compound.

Q^{b4} is an alkylene group, and the alkylene group may have —O—, —C(O)N(R^{d})— (the definition of R^{d} is the same as that described above), a silphenylene skeleton group, a divalent organopolysiloxane group, or a dialkylsilylene group.

When the alkylene group has —O— or a silphenylene skeleton group, the alkylene group preferably has —O— or a silphenylene skeleton group between carbon atoms. When the alkylene group has —C(O)N(R^{d})—, a dialkylsilylene group, or a divalent organopolysiloxane group, it is preferable to have these groups between carbon atoms or at the terminal on the side which is bonded to (O)ᵤ₄.

The number of carbon atoms of the alkylene group represented by Q^{b4} is preferably from 1 to 30, more preferably from 1 to 20, further preferably from 2 to 20, may be from 2 to 10, and may be from 2 to 6. The number of carbon atoms may be, for example, 2, 3, 8, 9, or 11. Further, the number of carbon atoms may be from 1 to 10.

u4 is 0 or 1.

As —(O)ᵤ₄-Q^{b4}-, —CH₂CH₂—, —CH₂CH₂CH₂—, —CH₂OCH₂CH₂CH₂—, —CH₂OCH₂CH₂CH₂CH₂CH₂—, —OCH₂CH₂CH₂—, —OSi(CH₃)₂CH₂CH₂CH₂—, —OSi(CH₃)₂OSi(CH₃)₂CH₂CH₂CH₂—, and —CH₂CH₂CH₂Si(CH₃)₂PhSi(CH₃)₂CH₂CH₂— are preferable from the viewpoint of the ease of manufacturing of the compound (note that the right side is bonded to Si).

w1 is an integer from 0 to 2, preferably 0 or 1, and particularly preferably 0.

In a case where there are two or more units of [—(O)ᵤ₄-Q^{b4}-Si(R¹)ₙL₃₋ₙ], two or more units of [—(O)ᵤ₄-Q^{b4}-Si(R¹)ₙL₃₋ₙ] may be the same as each other or different from each other.

In a case where there are two or more units of R³¹, two or more units of (—R³¹) may be the same as each other or different from each other.

In a case where the terminal of Z¹ on the side bonded to A¹¹ is an alkylene group, specific examples of the Group (3-1A-4) include the following groups. In the following formula, * represents a bonding position with Z¹.

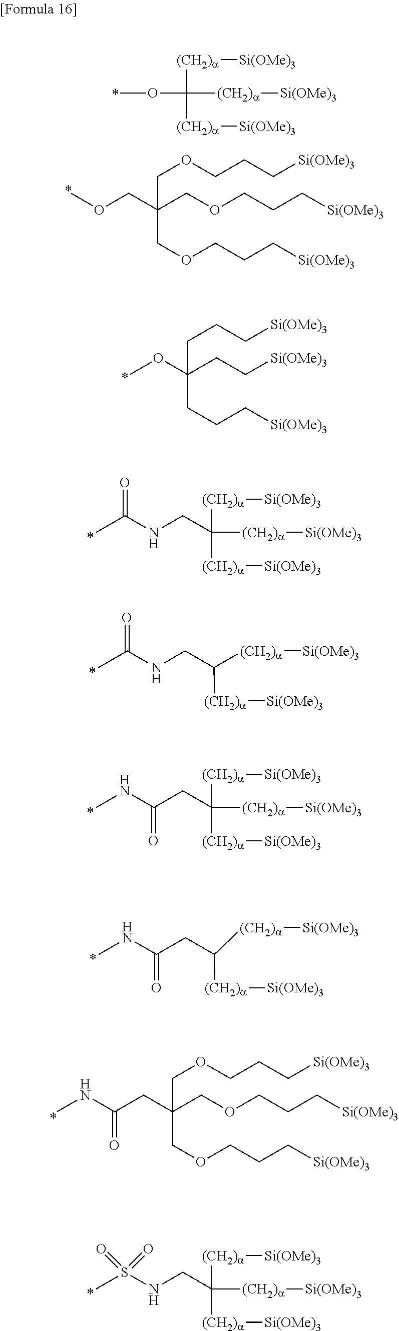

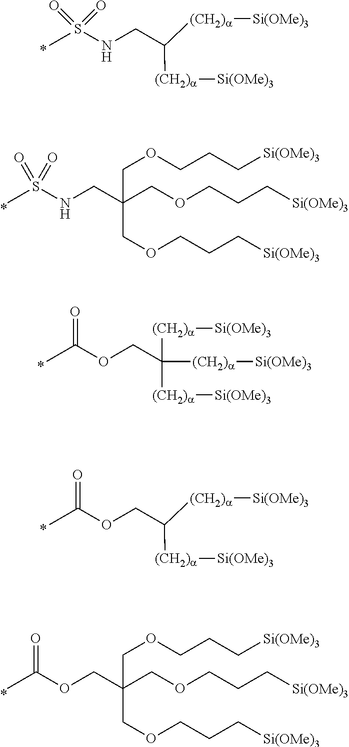

In a case where Z¹ is a polyalkylene oxide group, specific examples of the Group (3-1A-4) include the following groups. In the following formula, * represents a bonding position with Z¹.

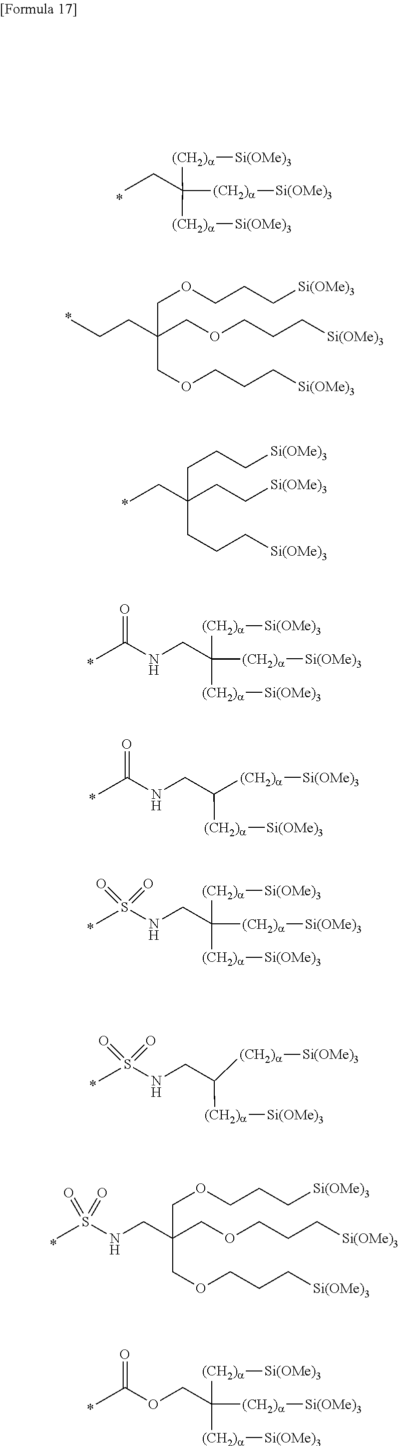

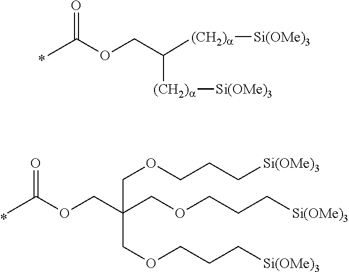

In the Group (3-1A-5), Q^{a5} is an alkylene group which may have an etheric oxygen atom.

The number of carbon atoms of the alkylene group which may have an etheric oxygen atom is preferably from 1 to 10, and particularly preferably from 2 to 6.

As Q^{a5}, from the viewpoint of the ease of manufacturing of the compound, —OCH₂CH₂CH₂—, —OCH₂CH₂OCH₂CH₂CH₂—, —CH₂CH₂—, and —CH₂CH₂CH₂— are preferable (note that the right side is bonded to Si).

Q^{b5} is an alkylene group, or a group having an etheric oxygen atom or a divalent organopolysiloxane group between carbon atoms of the alkylene group having a number of carbon atoms of 2 or more.

The number of carbon atoms of the alkylene group represented by Q^{b5} is preferably from 1 to 30, more preferably from 1 to 20, further preferably from 2 to 20, may be from 2 to 10, and may be from 2 to 6. The number of carbon atoms may be, for example, 2, 3, 8, 9, or 11. Further, the number of carbon atoms may be from 1 to 10.

The number of carbon atoms of the group having an etheric oxygen atom or a divalent organopolysiloxane group between carbon atoms of the alkylene group having a number of carbon atoms of 2 or more represented by Q^{b5} is preferably from 2 to 20, more preferably from 2 to 10, and further preferably from 2 to 6.

Q^{b5} is preferably —CH₂CH₂CH₂— or —CH₂CH₂OCH₂CH₂CH₂— from the viewpoint of the ease of manufacturing of the compound (note that the right side is bonded to Si(R¹)ₙL₃₋ₙ).

Three units of [-Q^{b5}-Si(R¹)ₙL₃₋ₙ] may be the same as each other or different from each other.

In a case where the terminal of Z¹ on the side bonded to A¹¹ is an alkylene group, specific examples of the Group (3-1A-5) include the following groups. In the following formula, * represents a bonding position with Z¹.

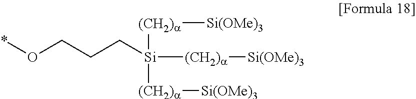

In a case where Z¹ is a polyalkylene oxide group, specific examples of the Group (3-1A-5) include the following groups. In the following formula, * represents a bonding position with Z¹.

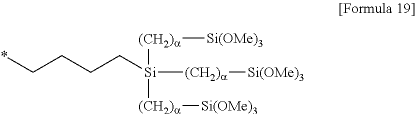

The definition of Q^{e} in Group (3-1A-6) is as defined in the above Group (3-1A-4).

v is 0 or 1.

Q^{a6} is an alkylene group which may have an etheric oxygen atom.

The number of carbon atoms of the alkylene group which may have an etheric oxygen atom is preferably from 1 to 10, and particularly preferably from 2 to 6.

Q^{a6} is preferably —CH₂OCH₂CH₂CH₂—, —CH₂OCH₂CH₂OCH₂CH₂CH₂—, —CH₂CH₂—, - or CH₂CH₂CH₂— from the viewpoint of the ease of manufacturing of the compound (note that the right side is bonded to Z^{a}).

Z^{a} is a (w2+1)-valent organopolysiloxane group or a (w2+1)-valent group having an alkylene group between the organopolysiloxane group and the organopolysiloxane group.

w2 is an integer from 2 to 7.

Examples of the (w2+1)-valent organopolysiloxane group and the (w2+1)-valent group having an alkylene group between the organopolysiloxane group and the organopolysiloxane group include the following groups. However, R^{a} in the following formula is as described above. * represents a bonding position.

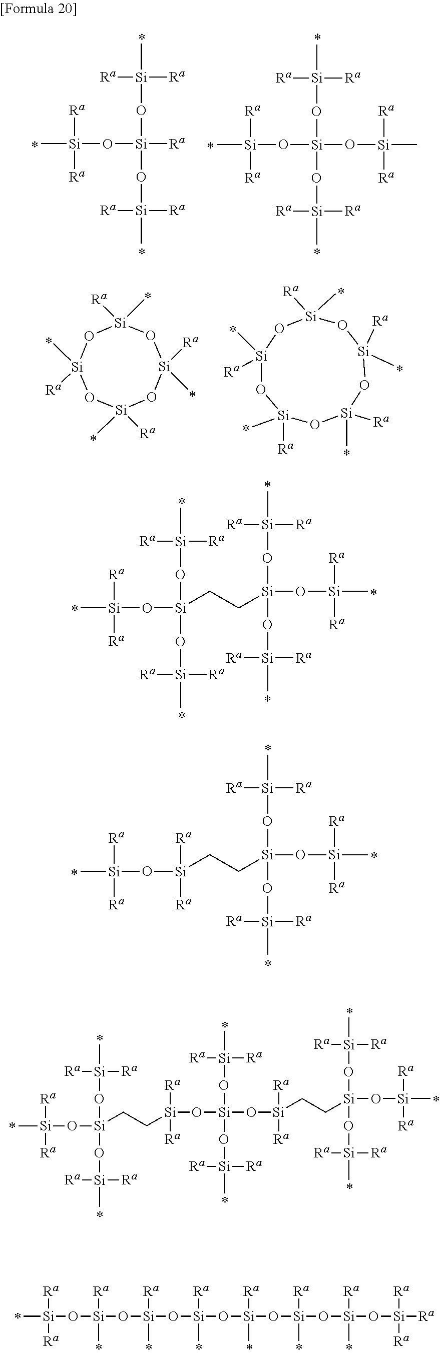

Q^{b6} is an alkylene group, or a group having an etheric oxygen atom or a divalent organopolysiloxane group between carbon atoms of the alkylene group having a number of carbon atoms of 2 or more.

The number of carbon atoms of the alkylene group represented by Q^{b6} is preferably from 1 to 30, more preferably from 1 to 20, further preferably from 2 to 20, may be from 2 to 10, and may be from 2 to 6. The number of carbon atoms may be, for example, 2, 3, 8, 9, or 11. Further, the number of carbon atoms may be from 1 to 10.

The number of carbon atoms of the group having an etheric oxygen atom or a divalent organopolysiloxane group between carbon atoms of the alkylene group having a number of carbon atoms of 2 or more represented by Q^{b6} is preferably from 2 to 20, more preferably from 2 to 10, and further preferably from 2 to 6.

Q^{b6} is preferably —CH₂CH₂— or —CH₂CH₂CH₂— from the viewpoint of the ease of manufacturing of the compound.

w2 units of [-Q^{b6}-Si(R¹)ₙL₃₋ₙ] may be the same as each other or different from each other.

Specific examples of the Group (3-1A-6) include the following groups. In the following formula, * represents a bonding position with Z¹.

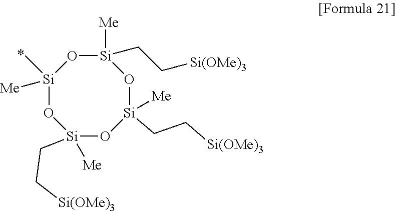

In the Group (3-1A-7), Z^{e} is a (w3+w4+1)-valent hydrocarbon group.

w3 is an integer of 4 or more.

w4 is an integer of 0 or more.

The definitions and preferred ranges of Q^{e}, s4, Q^{a4}, t4, Q^{b4}, and u4 are the same as the definitions of the respective reference symbols in the Group (3-1A-4).

Z^{e} may consist of a hydrocarbon chain, may have an etheric oxygen atom between carbon atoms of a hydrocarbon chain, and preferably consists of a hydrocarbon chain.

The valence of Z^{e} is preferably from 5 to 20, more preferably from 5 to 10, still more preferably from 5 to 8, and particularly preferably from 5 to 6.

The number of carbon atoms of Z^{e} is preferably from 3 to 50, more preferably from 4 to 40, and further preferably from 5 to 30.

w3 is preferably from 4 to 20, more preferably from 4 to 16, further preferably from 4 to 8, and particularly preferably from 4 to 5.

w4 is preferably from 0 to 10, more preferably from 0 to 8, further preferably from 0 to 6, particularly preferably from 0 to 3, and most preferably from 0 to 1.

In a case where there are two or more units of [—(O-Q^{b4})ᵤ₄-Si(R¹)ₙL₃₋ₙ], the two or more units of [—(O-Q^{b4})ᵤ₄-Si(R¹)ₙL₃₋ₙ] may be the same as each other or different from each other.

In a case where the terminal of Z¹ on the side of A¹¹ is an alkylene group, specific examples of the Group (3-1A-7) include the following groups. In the following formula, * represents a bonding position with Z¹.

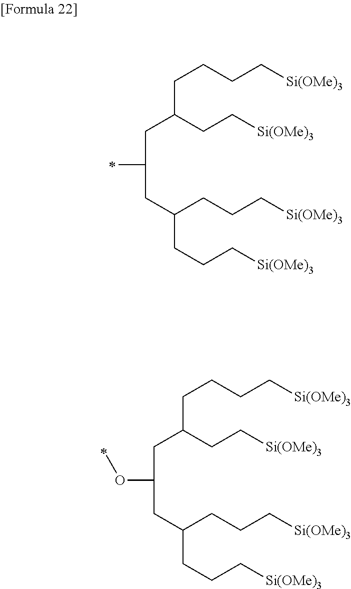

In a case where the terminal of Z¹ on the side of A¹¹ is a polyalkylene oxide group, specific examples of the Group (3-1A-7) include the following groups. In the following formula, * represents a bonding position with Z¹.

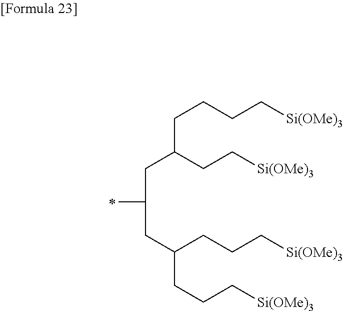

A¹¹ may be a Group (g2-1) (where j1=d1+d3, g1=d2+d4), a Group (g2-2) (where j1=e1, g1=e2), a Group (g2-3) (where j1=1, g1=2), a Group (g2-4) (where j1=h1, g1=h2), a Group (g2-5) (where j1=i1, g1=i2), a Group (g2-6) (where j1=1, g1=1), or a Group (g2-7) (where j1=1, g1=i3).

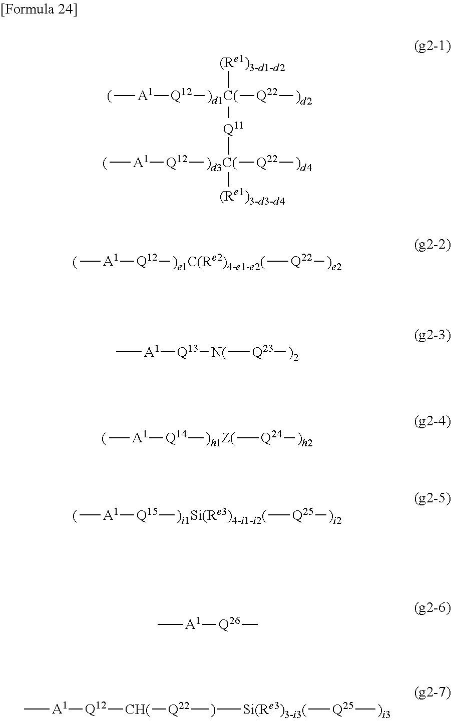

Note that in Formulas (g2-1) to (g2-7), the A¹ side is bonded to Z, and the Q²², Q²³, Q²⁴, Q²⁵ or Q²⁶ side is bonded to [—Si(R²)ₙL₃₋ₙ].

In a case where the terminal of Z on the side bonded to A¹ is an alkylene group, the terminal of A¹ on the side bonded to Z is not an alkylene group.

In a case where Z is a polyalkylene oxide group, the terminal of A¹ on the side bonded to Z is not an alkylene oxide group.

A¹ is a single bond, —C(O)NR⁶—, —C(O)—, —OC(O)O—, —NHC(O)O—, —NHC(O)NR⁶—, —O—, or —SO₂NR⁶—.

Q¹¹ is a single bond, —O—, an alkylene group, or a group having —C(O)NR⁶—, —C(O)—, —NR⁶—, or —O— between carbon atoms of an alkylene group having a number of carbon atoms of 2 or more.

Q¹² is a single bond, an alkylene group, or a group having —C(O)NR⁶—, —C(O)—, —NR⁶—, or O— between carbon atoms of an alkylene group having a number of carbon atoms of 2 or more, and when A¹¹ has two or more units of Q¹², two or more units of Q¹² may be the same as each other or different from each other.

Q¹³ is a single bond (where A¹ is —C(O)—), an alkylene group, a group having —C(O)NR⁶—, —C(O)—, —NR⁶—, or O— between carbon atoms of an alkylene group having a number of carbon atoms of 2 or more, or a group having —C(O)— at an N-side terminal of an alkylene group.

Q¹⁴ is Q¹² when the atom in Z to which Q¹⁴ is bonded is a carbon atom, Q¹⁴ is Q¹³ when the atom in Z to which Q¹⁴ is bonded is a nitrogen atom, and when A¹¹ has two or more units of Q¹⁴, two or more units of Q¹⁴ may be the same as each other or different from each other.

Q¹⁵ is an alkylene group, or a group having —C(O)NR⁶—, —C(O)—, —NR⁶—, or O— between carbon atoms of an alkylene group having a number of carbon atoms of 2 or more, and when A¹¹ has two or more units of Q¹⁵, two or more units of Q¹⁵ may be the same as each other or different from each other.

Q²² is an alkylene group, a group having —C(O)NR⁶—, —C(O)—, —NR⁶—, or O— between carbon atoms of an alkylene group having a number of carbon atoms of 2 or more, a group having —C(O)NR⁶—, —C(O)—, —NR⁶—, or O— at a terminal of the alkylene group on a side which is not connected to Si, or a group having —C(O)NR⁶—, —C(O)—, —NR⁶—, or O— between carbon atoms of an alkylene group having a number of carbon atoms of 2 or more and having —C(O)NR⁶—, —C(O)—, —NR⁶—, or O— at a terminal on a side which is not connected to Si, and when A¹¹ has two or more units of Q²², two or more units of Q²² may be the same as each other or different from each other.

Q²³ is an alkylene group, or a group having —C(O)NR⁶—, —C(O)—, —NR⁶—, or O— between carbon atoms of an alkylene group having a number of carbon atoms of 2 or more, and two units of Q²³ may be the same as each other or different from each other.

Q²⁴ is Q²² when the atom in Z to which Q²⁴ is bonded is a carbon atom, Q²³ when the atom in Z to which Q²⁴ is bonded is a nitrogen atom, and when A¹¹ has two or more units of Q²⁴, two or more units of Q²⁴ may be the same as each other or different from each other.

Q²⁵ is an alkylene group, or a group having —C(O)NR⁶—, —C(O)—, —NR⁶—, or O— between carbon atoms of an alkylene group having a number of carbon atoms of 2 or more, and when A¹¹ has two or more units of Q²⁵, two or more units of Q²⁵ may be the same as each other or different from each other.

Q²⁶ is an alkylene group, or a group having —C(O)NR⁶—, —C(O)—, —NR⁶—, or O— between carbon atoms of an alkylene group having a number of carbon atoms of 2 or more.

In a case where Q²², Q²³, Q²⁴, Q²⁵, and Q²⁶ are alkylene groups, the number of carbon atoms is preferably from 1 to 20, more preferably from 1 to 10, and further preferably from 1 to 6.

Z is a group having a (h1+h2)-valent ring structure having a carbon atom or a nitrogen atom to which Q¹⁴ is directly bonded and having a carbon atom or a nitrogen atom to which Q²⁴ is directly bonded.

R^{e1} is a hydrogen atom or an alkyl group, and when A¹¹ has two or more units of Re, two or more units of R^{e1} may be the same as each other or different from each other.

R^{e2} is a hydrogen atom, a hydroxyl group, an alkyl group, or an acyloxy group.

R^{e3} is an alkyl group. R⁶ is a hydrogen atom, an alkyl group having a number of carbon atoms from 1 to 6, or a phenyl group.

d1 is an integer from 0 to 3, and preferably 1 or 2.

d2 is an integer from 0 to 3, and preferably 1 or 2.

d1+d2 is an integer from 1 to 3.

d3 is an integer from 0 to 3, and preferably 0 or 1.

d4 is an integer from 0 to 3, and preferably 2 or 3.

d3+d4 is an integer from 1 to 3.

d1+d3 is an integer from 1 to 5, and preferably 1 or 2.

d2+d4 is an integer from 1 to 5, and preferably 4 or 5.

e1+e2 is 3 or 4.

e1 is an integer from 1 to 3, and preferably 1 or 2.

e2 is an integer from 1 to 3, and preferably 2 or 3.

h1 is an integer of 1 or more, and preferably 1 or 2.

h2 is an integer of 1 or more, and preferably 2 or 3.

i1+i2 is 3 or 4.

i1 is an integer from 1 to 3, and preferably 1 or 2.

i2 is an integer from 1 to 3, and preferably 2 or 3.

i3 is 2 or 3.

The number of carbon atoms of the alkylene groups of Q¹¹, Q¹², Q¹³, Q¹⁴, Q¹⁵, Q²², Q²³, Q²⁴, Q²⁵, and Q²⁶ are preferably from 1 to 30, more preferably from 1 to 20, further preferably from 2 to 20, may be from 2 to 10, and may be from 2 to 6 from the viewpoint of the ease of manufacturing of the compound and because the abrasion resistance of the surface treatment layer becomes more excellent. The number of carbon atoms may be, for example, 2, 3, 8, 9, and 11. The number of carbon atoms may be from 1 to 10, from 1 to 6, or from 1 to 4. However, when the alkylene group has a specific bond between carbon atoms, the lower limit value of its number of carbon atoms is 2.

Examples of the ring structure in Z include the ring structures described above, and preferred forms are also the same.

Q¹⁴ and Q²⁴ are directly bonded to the ring structure in Z, and thus, for example, there is no such a case where an alkylene group is linked to the ring structure and Q¹⁴ and Q²⁴ are linked to the alkylene group.

The number of carbon atoms of the alkyl group of R^{e1}, R^{e2}, or R^{e3} is preferably from 1 to 6, more preferably from 1 to 3, and particularly preferably from 1 to 2 from the viewpoint of the ease of manufacturing of the compound.

The number of carbon atoms of the alkyl group moiety of the acyloxy group of R^{e2} is preferably from 1 to 6, more preferably from 1 to 3, and particularly preferably from 1 to 2 from the viewpoint of the ease of manufacturing of the compound.

h1 is preferably from 1 to 6, more preferably from 1 to 4, further preferably 1 or 2, and particularly preferably 1 from the viewpoint of the ease of manufacturing of the compound and because the abrasion resistance of the surface treatment layer becomes more excellent.

h2 is preferably from 2 to 6, more preferably from 2 to 4, and particularly preferably 2 or 3 from the viewpoint of the ease of manufacturing of the compound and because the abrasion resistance of the surface treatment layer becomes more excellent.

Other forms of A¹¹ include a Group (g2-8) (where j1=d1+d3, g1=d2×k3+d4×k3), a Group (g2-9) (where j1=e1, g1=e2×k3), a Group (g2-10) (where j1=1, g1=2×k3), a Group (g2-11) (where j1=h1, g1=h2×k3), a Group (g2-12) (where j1=i1, g1=i2×k3), a Group (g2-13) (where j1=1, g1=k3), or a Group (g2-14) (where j1=1, g1=i3×k3).

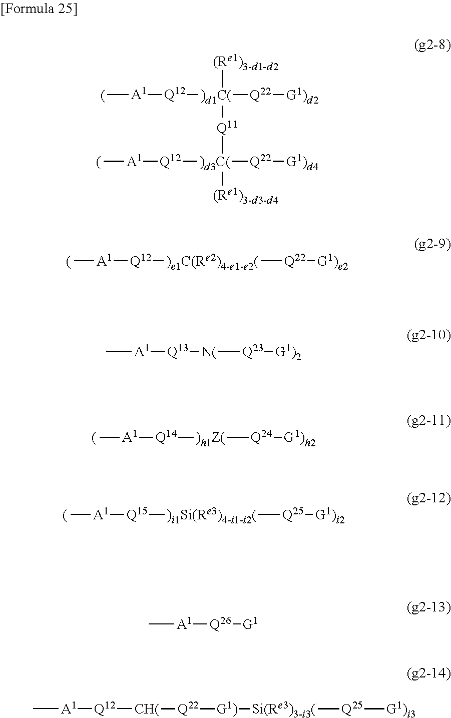

In Formulas (g2-8) to (g2-14), the A¹ side is bonded to Z¹ and the G¹ side is bonded to [—Si(R²)ₙL₃₋ₙ].

In a case where the terminal of Z¹ on the side bonded to A¹ is an alkylene group, the terminal of A¹ on the side bonded to Z¹ is not an alkylene group.

In a case where Z¹ is a polyalkylene oxide group, the terminal of A¹ on the side bonded to Z¹ is not an alkylene oxide group.

G¹ is the following Group (g3), and two or more units of G¹ of A¹¹ may be the same as each other or different from each other. The reference symbols other than G¹ are the same as the reference symbols in Formulas (g2-1) to (g2-7):

—Si(R⁸)₃₋ₖ₃(-Q³-)ₖ₃ (g3)
where in the Group (g3), the Si side is connected to Q²², Q²³, Q²⁴, Q²⁵, and Q²⁶, and the Q³ side is connected to [—Si(R²)ₙL₃₋ₙ]. R⁸ is an alkyl group. Q³ is an alkylene group, a group having —C(O)NR⁶—, —C(O)—, —NR⁶— or —O— between carbon atoms of an alkylene group having a number of carbon atoms of 2 or more, or (OSi(R⁹)₂)ₚ—O—, and two or more units of Q³ may be the same as each other or different from each other. k3 is 2 or 3. R⁶ is a hydrogen atom, an alkyl group having a number of carbon atoms from 1 to 6, or a phenyl group. R⁹ is an alkyl group, a phenyl group, or an alkoxy group, and two units of R⁹ may be the same as each other or different from each other. p is an integer from 0 to 5, and when p is 2 or more, 2 or more (OSi(R⁹)₂) may be the same as each other or different from each other.

The number of carbon atoms of the alkylene group of Q³ is preferably from 1 to 30, more preferably from 1 to 20, further preferably from 2 to 20, may be from 2 to 10, and may be from 2 to 6 from the viewpoint of the ease of manufacturing of the compound and because the abrasion resistance of the surface treatment layer becomes more excellent. The number of carbon atoms may be, for example, 2, 3, 8, 9, or 11. Further, the number of carbon atoms may be from 1 to 10, may be from 1 to 6, or may be from 1 to 4. However, when the alkylene group has a specific bond between carbon atoms, the lower limit value of its number of carbon atoms is 2.

The number of carbon atoms of the alkyl group of R⁸ is preferably from 1 to 6, more preferably from 1 to 3, and further preferably from 1 to 2 from the viewpoint of the ease of manufacturing of the compound.

The number of carbon atoms of the alkyl group of R⁹ is preferably from 1 to 6, more preferably from 1 to 3, and further preferably from 1 to 2 from the viewpoint of the ease of manufacturing of the compound.

The number of carbon atoms of the alkoxy group of R⁹ is preferably from 1 to 6, more preferably from 1 to 3, and particularly preferably from 1 to 2 from the viewpoint of more excellent storage stability of the compound.

p is preferably 0 or 1.

Examples of the compound according to the present disclosure include a compound of the following Formula. The compound of the following formula is preferable from the viewpoint of easy industrial production, easy handling, and being further excellent in water repellency and abrasion resistance of the surface treatment layer. R^{t} in the compound of the following formula is the same as [R¹¹-(Q)ᵣ₁-(M¹¹)ᵣ₁₁-(Q)ᵣ₂-Z¹] in Formula (1a), and a preferable form is also the same.

In a case where the terminal of Z¹ on the side bonded to A¹¹ is an alkylene group, examples of the compound in which A¹¹ in Formula 1 is a Group (g2-1) include a compound of the following Formula.

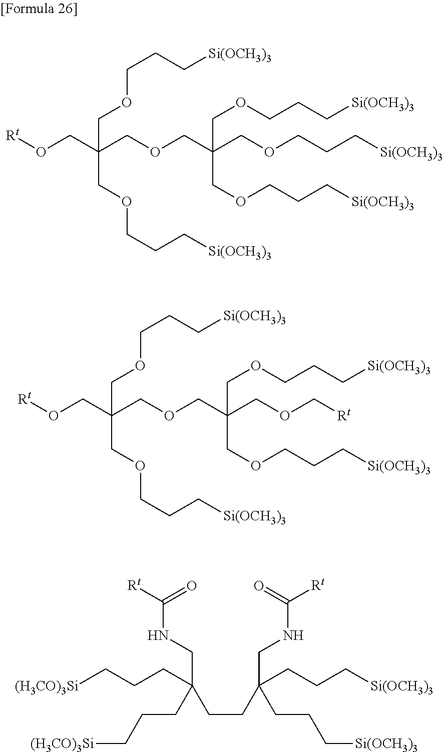

In a case where Z¹ is a polyalkylene oxide group, examples of the compound in which A¹¹ in Formula 1 is a Group (g2-1) include a compound of the following Formula.

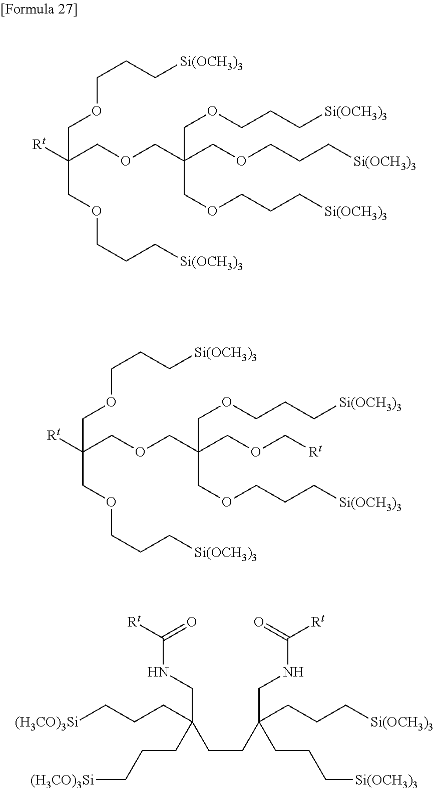

In a case where the terminal of Z¹ on the side bonded to A¹¹ is an alkylene group, examples of the compound in which A¹¹ in Formula 1 is a Group (g2-2) include a compound of the following Formula. In the following Formula, Ak means an alkyl group (e.g., an alkyl group having a number of carbon atoms from 1 to 15).

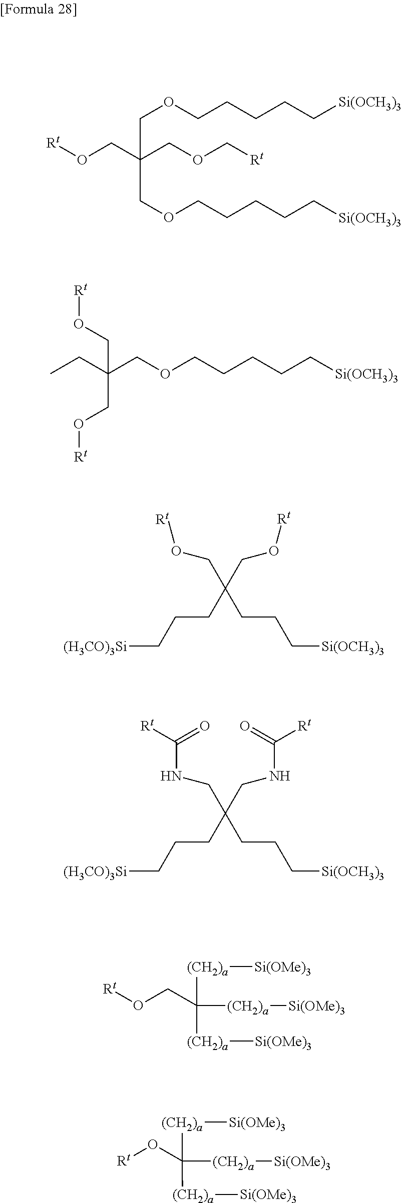

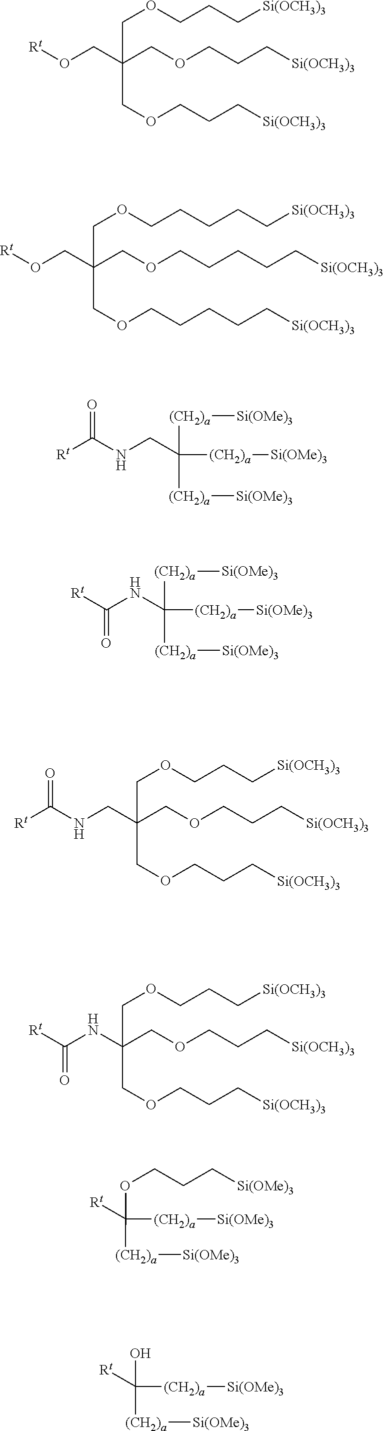

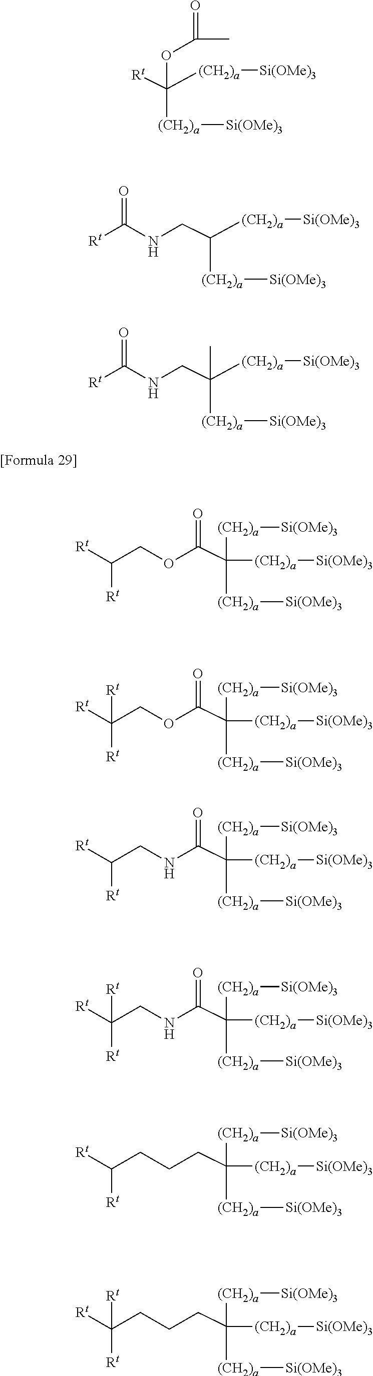

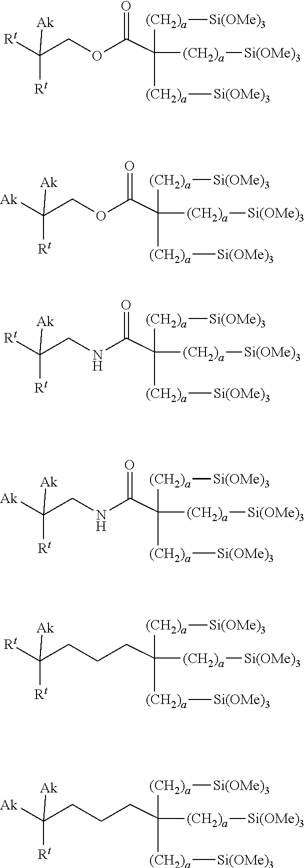

In a case where Z¹ is a polyalkylene oxide group, examples of the compound in which A¹¹ in Formula (1a) is a Group (g2-2) include a compound of the following Formula.

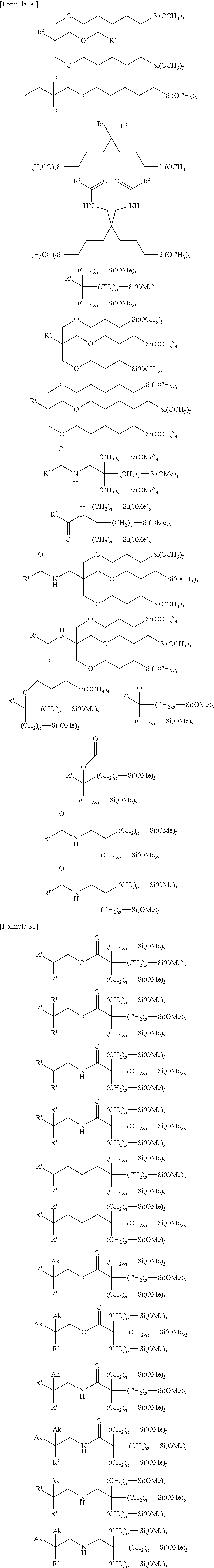

In a case where the terminal of Z¹ on the side bonded to A¹¹ is an alkylene group, examples of the compound in which A¹¹ in Formula 1 is a Group (g2-3) include a compound of the following Formula.

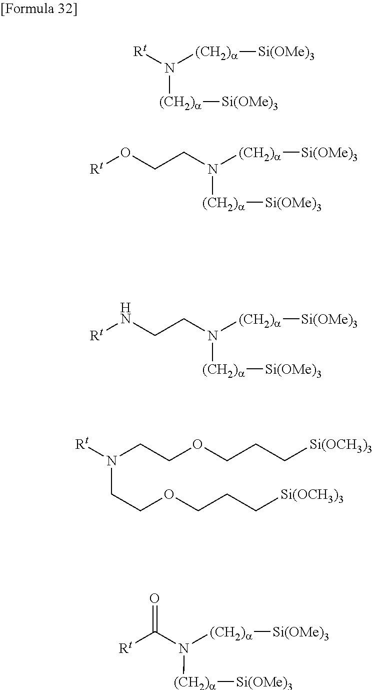

In a case where Z¹ is a polyalkylene oxide group, examples of the compound in which A¹¹ in Formula 1 is a Group (g2-3) include a compound of the following Formula.

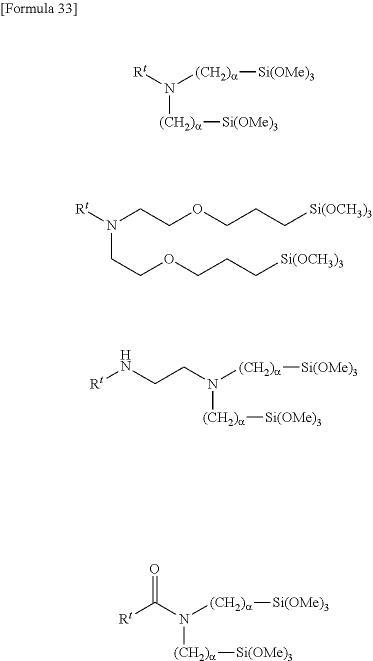

In a case where the terminal of Z¹ on the side bonded to A¹¹ is an alkylene group, examples of the compound in which A¹¹ in Formula 1 is a Group (g2-4) include a compound of the following Formula.

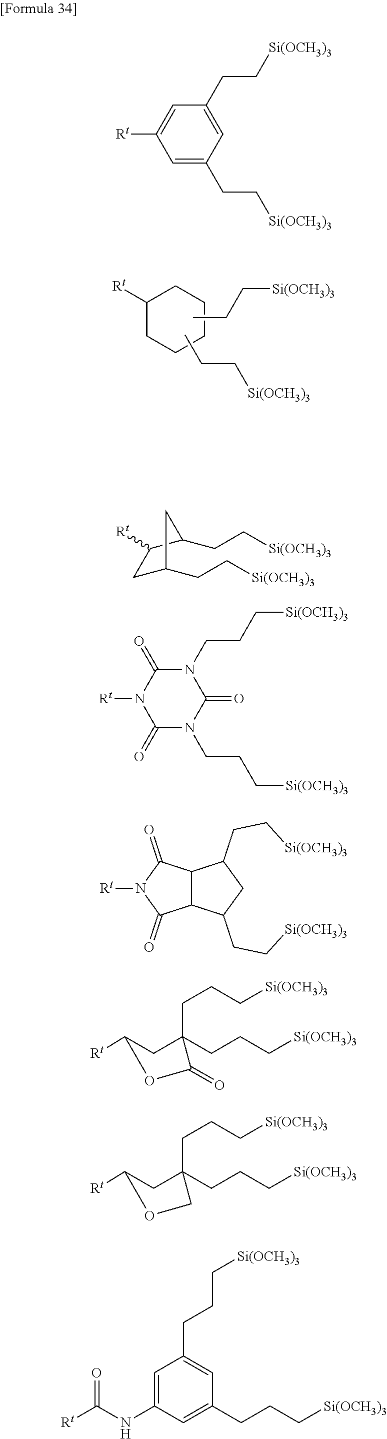

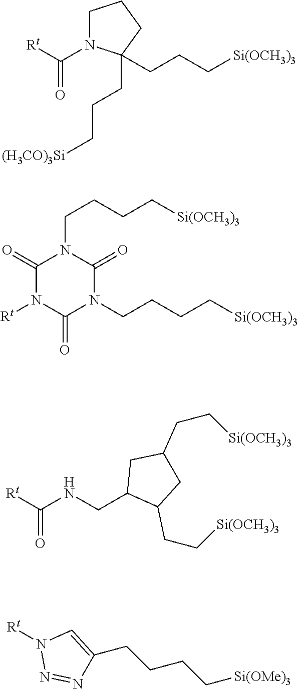

In a case where Z¹ is a polyalkylene oxide group, examples of the compound in which A¹¹ in Formula 1 is a Group (g2-4) include a compound of the following Formula.

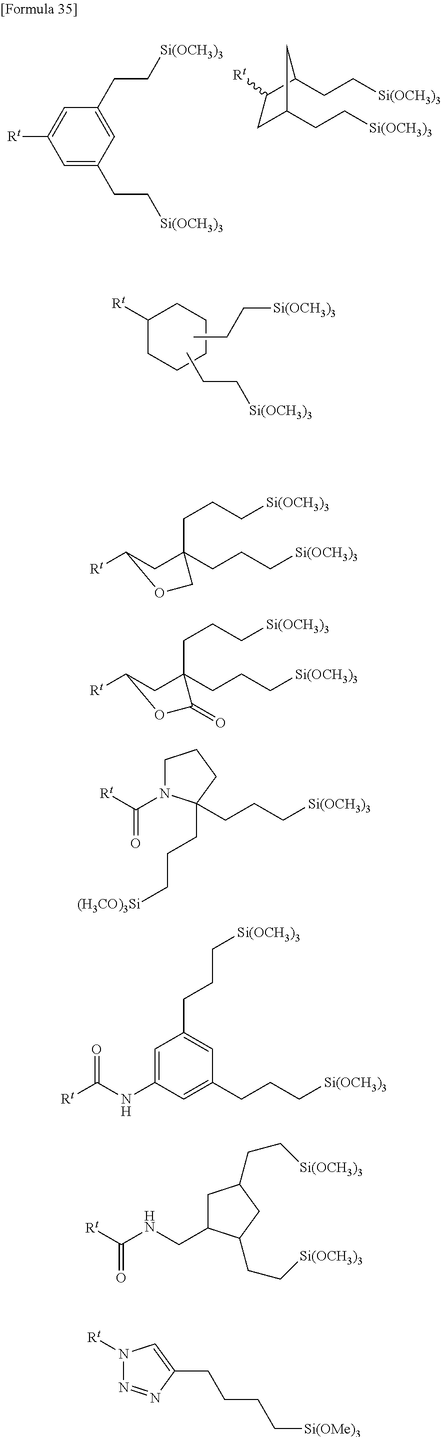

In a case where the terminal of Z¹ on the side bonded to A¹¹ is an alkylene group, examples of the compound in which A¹¹ in Formula 1 is a Group (g2-5) include a compound of the following Formula.

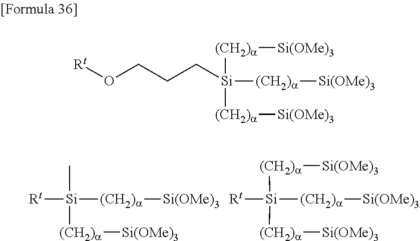

In a case where Z¹ is a polyalkylene oxide group, examples of the compound in which A¹¹ in Formula 1 is a Group (g2-5) include a compound of the following Formula.

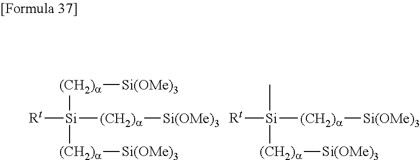

In a case where the terminal of Z¹ on the side bonded to A¹¹ is an alkylene group, examples of the compound in which A¹ in Formula 1 is a Group (g2-6) include a compound of the following Formula.

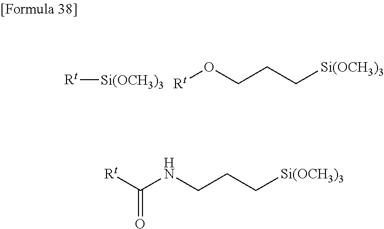

In a case where Z¹ is a polyalkylene oxide group, examples of the compound in which A¹¹ in Formula 1 is a Group (g2-6) include a compound of the following Formula.

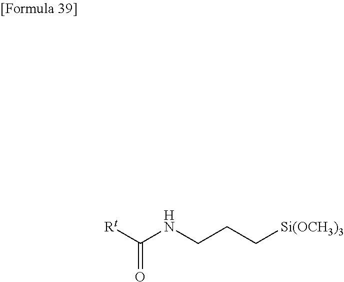

In a case where the terminal of Z¹ on the side bonded to A¹¹ is an alkylene group, examples of the compound in which A¹¹ in Formula 1 is a Group (g2-7) include a compound of the following Formula.

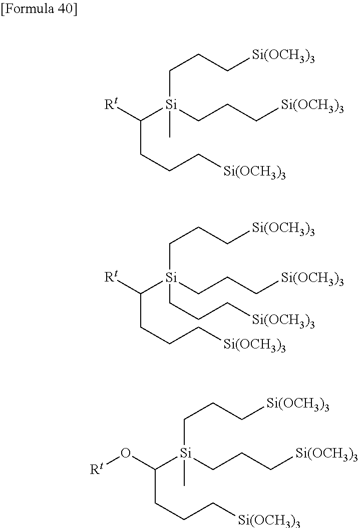

In a case where Z¹ is a polyalkylene oxide group, examples of the compound in which A¹¹ in Formula 1 is a Group (g2-7) include a compound of the following Formula.

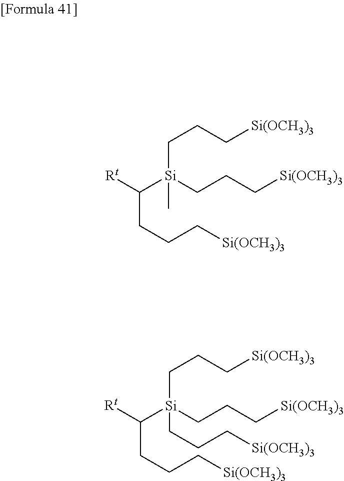

In a case where the terminal of Z¹ on the side bonded to A¹¹ is an alkylene group, examples of the compound in which A¹¹ in Formula 1 is a Group (g2-8) include a compound of the following Formula.

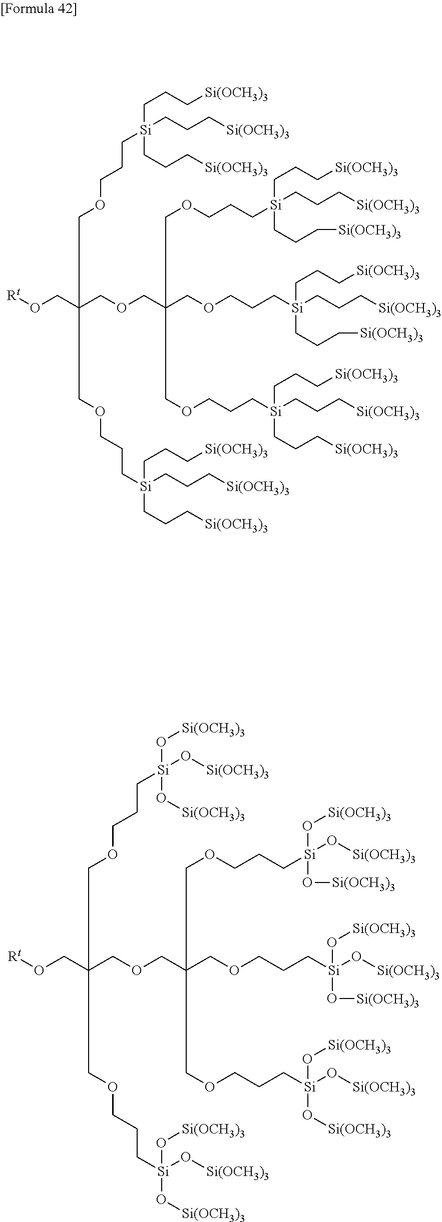

In a case where Z¹ is a polyalkylene oxide group, examples of the compound in which A¹¹ in Formula 1 is a Group (g2-8) include a compound of the following Formula.

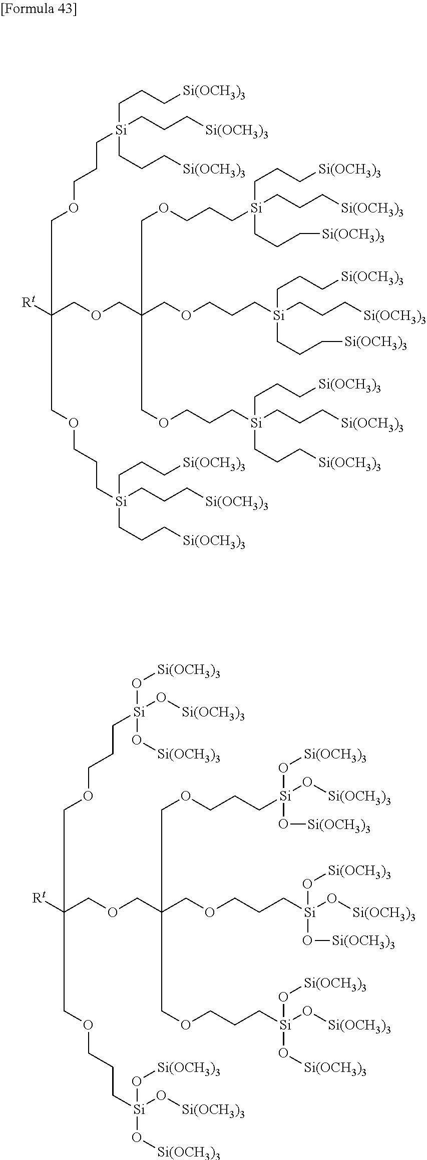

Examples of the compound in which A¹¹ in Formula 1 is a Group (g2-9) include a compound of the following Formula.

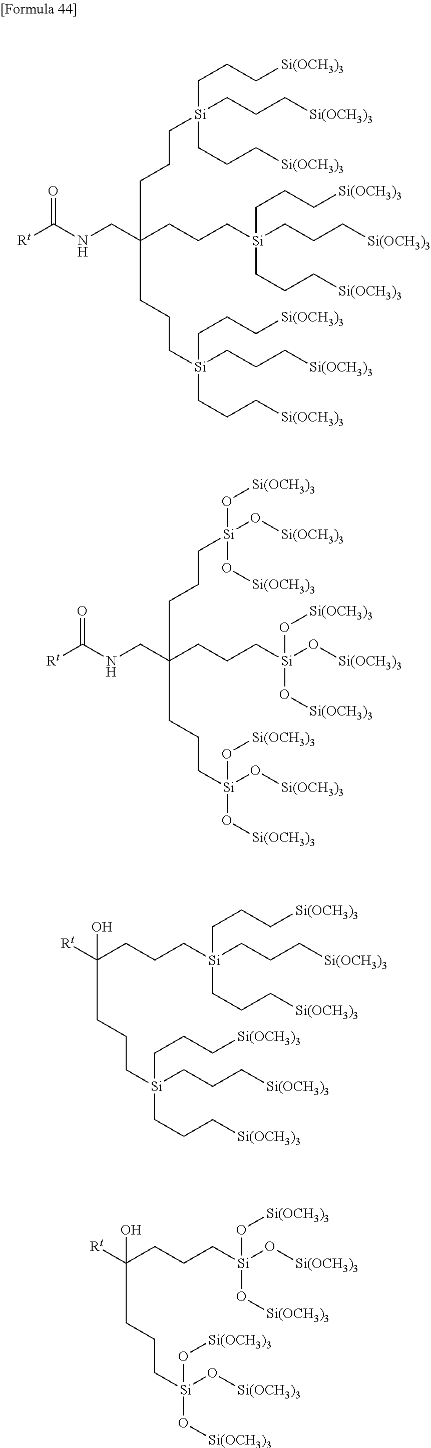

In a case where the terminal of Z¹ on the side bonded to A¹¹ is an alkylene group, examples of the compound in which A¹¹ in Formula 1 is a Group (g2-10) include a compound of the following Formula.

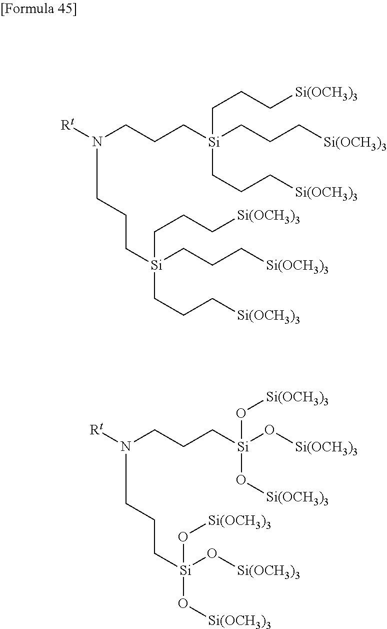

In a case where Z¹ is a polyalkylene oxide group, examples of the compound in which A¹¹ in Formula 1 is a Group (g2-10) include a compound of the following Formula.

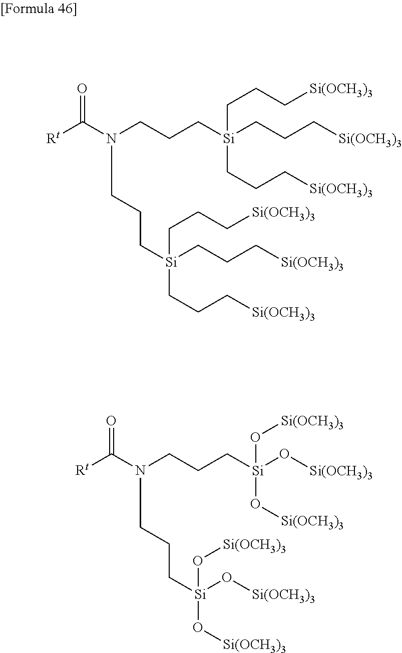

In a case where the terminal of Z¹ on the side bonded to A¹¹ is an alkylene group, examples of the compound in which A¹¹ in Formula 1 is a Group (g2-11) include a compound of the following Formula.

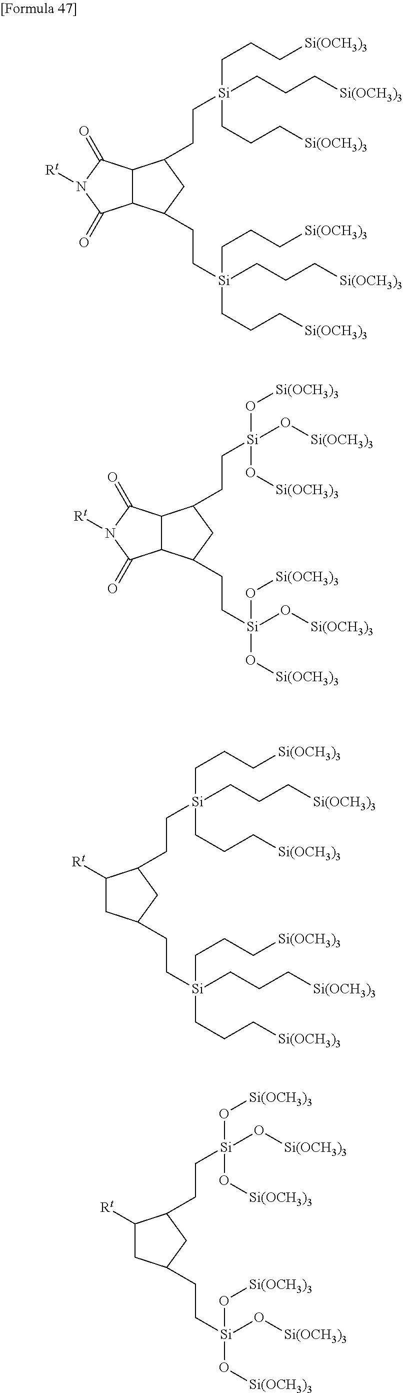

In a case where Z¹ is a polyalkylene oxide group, examples of the compound in which A¹¹ in Formula 1 is a Group (g2-11) include a compound of the following Formula.

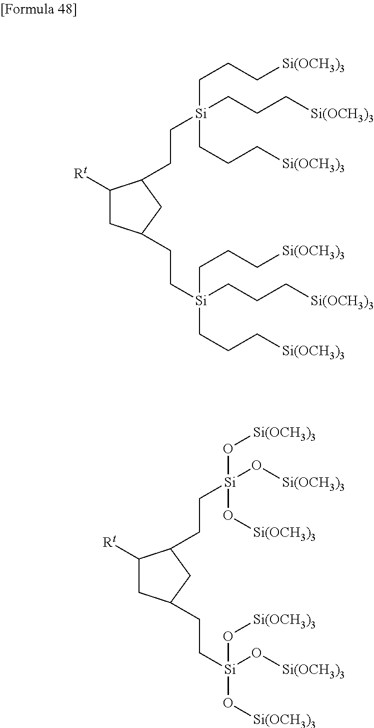

In a case where the terminal of Z¹ on the side bonded to A¹¹ is an alkylene group, examples of the compound in which A¹¹ in Formula 1 is a Group (g2-12) include a compound of the following Formula.

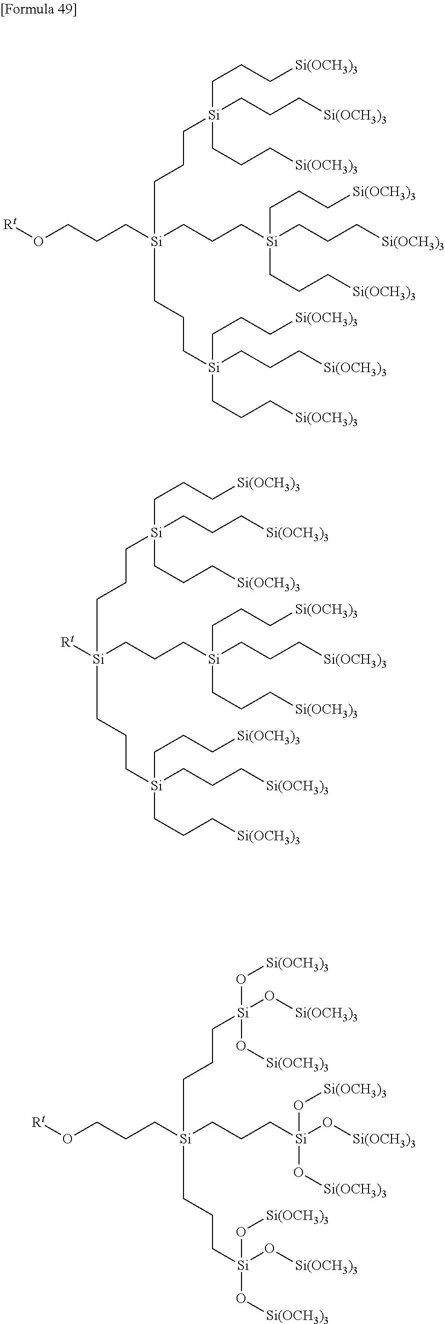

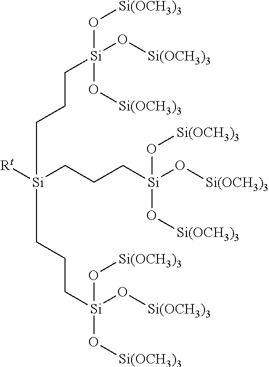

In a case where Z¹ is a polyalkylene oxide group, examples of the compound in which A¹¹ in Formula 1 is a Group (g2-12) include a compound of the following Formula.

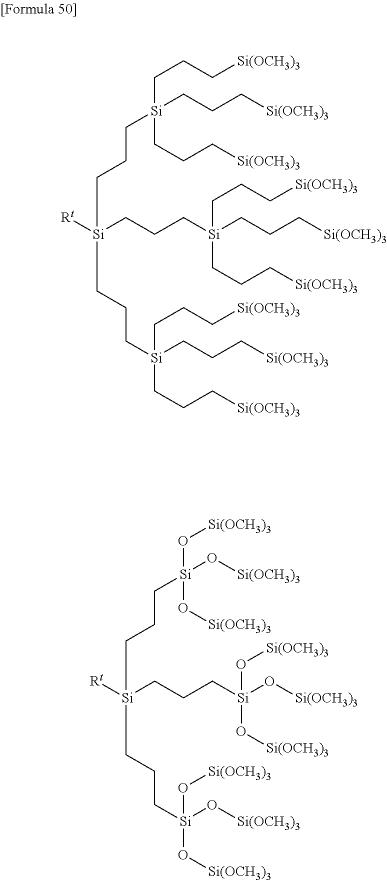

In a case where the terminal of Z¹ on the side bonded to A¹¹ is an alkylene group, examples of the compound in which A¹¹ in Formula 1 is a Group (g2-13) include a compound of the following Formula.

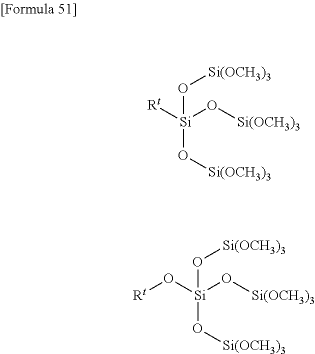

In a case where Z¹ is a polyalkylene oxide group, examples of the compound in which A¹¹ in Formula 1 is a Group (g2-13) include a compound of the following Formula.

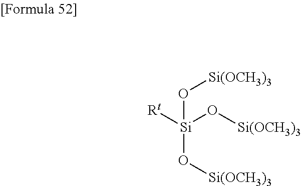

Examples of the compound in which A¹¹ in Formula 1 is a Group (g2-14) include a compound of the following Formula.

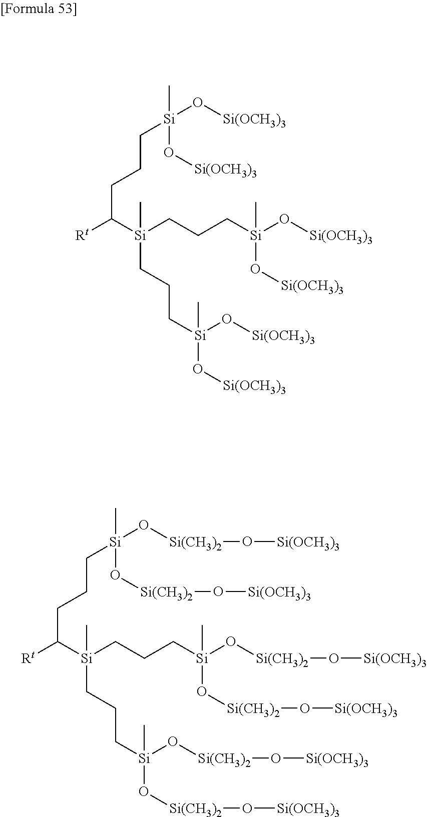

<Compound (1b), Compound (2b)>

In Formula (1a), r11 is 0 or r11 is 1, and M¹¹ is preferably Si, Sn, or Ge from the viewpoint of abrasion resistance and water repellency.

Further, in Formula (2a), r12 is preferably 0 from the viewpoint of abrasion resistance and water repellency. Among them, compounds represented by the following Formula (1b) or the following Formula (2b) are preferable from the viewpoint of abrasion resistance and water repellency:

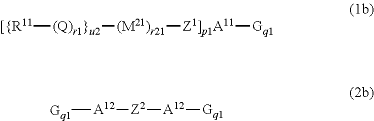

where
M²¹ is Si, Sn, or Ge,
r21 is 0 or 1,
u2 is 1 when r21 is 0 and is 3 when r21 is 1,
Z² is an organopolysiloxane group including Group 2, or a combination of an alkylene group with an organopolysiloxane group including the Group 2,
each of the other reference symbols is similar to each of reference symbols in Formulas (1a) and (2a), and
in Formula (1b), at least one of R¹¹ is Group 3, or at least one of R¹¹ or Z¹ is an organopolysiloxane group including Group 2.

The organopolysiloxane group including Group 2 in Z² is the same as that in the above Group (A1), and preferable forms thereof are also the same.

Examples of the combination of the alkylene group with the organopolysiloxane group including the Group 2 in Z² may include the Groups (B1) to (B4), and at least one of R^{Si} is the Group (A1). In Z², both * and ** in the Groups (B1) to (B4) are bonds with A¹². Among them, the combination of the alkylene group with the organopolysiloxane group including the Group 2 in Z² is preferably a Group (B3).

In the compounds (1a) and (1b), Z¹ is preferably a group represented by the following Formula (3):

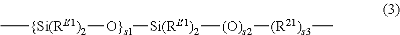

where R^{E1} is each independently a hydrocarbon group in which a hydrogen atom may be heavy hydrogen,
R²¹ is an alkylene group in which a hydrogen atom may be heavy hydrogen,
s1 is an integer from 0 to 500,
s2 is 0 or 1, and
s3 is 0 or 1.

Note that the Group (3) has one or more heavy hydrogen.

R^{E1} is the same as R^{E1} in the above Formula (A1), and preferable forms thereof are also the same. At least one of R^{E1} is preferably a hydrocarbon group including heavy hydrogen.

The number of carbon atoms of the alkylene group in R²¹ may be 1 or more, preferably 2 or more, and more preferably 4 or more. The upper limit of the number of carbon atoms of the alkylene group is not particularly limited, and is, for example, 30 or less, and preferably 24 or less. The alkylene group in Ak¹ is preferably linear or branched, and more preferably linear.

A group represented by the Formula (3) is further preferably a group represented by the following Formula (3A):

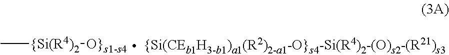

where R⁴ is a hydrocarbon group that does not include heavy hydrogen,
s4 is an integer from 0 to s1, and
each of the other reference symbols is similar to each of reference symbols in Formulas (1a) and (3).

Among them, s4 is preferably from 1 to 24, and more preferably from 1 to 12.

Further, in the compound (2b), Z² is preferably a group represented by the following Formula (4):

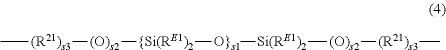

where each of the reference symbols is similar to those in the Formula (3).

A group represented by the Formula (4) is further preferably a group represented by the following Formula (4A):

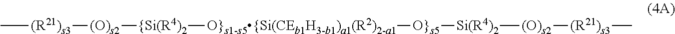

where R⁴ is a hydrocarbon group that does not include heavy hydrogen,
s5 is an integer from 0 to s1, and
each of the other reference symbols is similar to each of reference symbols in Formulas (1b) and (4).

Among them, s5 is preferably from 1 to 24, and more preferably from 1 to 12.

<Compound (1c), Compound (2c)>

The compound according to the present disclosure is preferably a compound represented by the following Formula (1c) or the following Formula (2c) from the viewpoint of abrasion resistance and water repellency:

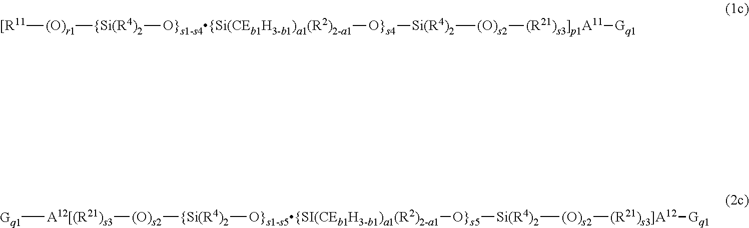

where each of the reference symbols in the Formulas is the same PGP as that described above.

Specific examples of the compound of the present disclosure include, but not limited to, the following compounds.

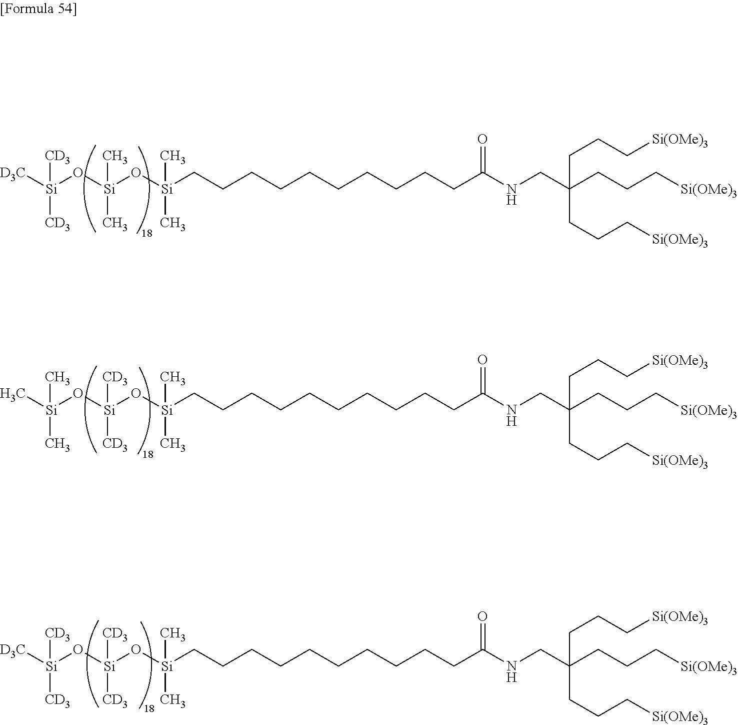

The number average molecular weight (Mn) of the compound according to the present disclosure is preferably from 500 to 20,000, more preferably from 600 to 18,000, and further preferably from 700 to 15,000.

When Mn is 500 or more, the abrasion resistance of the surface treatment layer is more excellent. When Mn is 20,000 or less, the viscosity is easily adjusted within an appropriate range, and the solubility is improved, and thus handleability at the time of film formation is excellent.

### [Method for Manufacturing Compound of Present Disclosure]

The aforementioned method for manufacturing the compound of the present disclosure is not particularly limited. For example, by adding dimethylchlorosilane to a compound having an unsaturated double bond to synthesize a compound represented by ClSi(Me)₂-R (where R is an organic group capable of introducing the Group 1), and causing Si(CH₃)OH or (—Si(CH₃)₂O—)ₙ to be reacted with the synthesized compound, organopolysiloxane can be introduced. By using Si(CD₃)₃₀H or (—Si(CD₃)₂O—)ₙ in this reaction, a compound having an organopolysiloxane group including Group 2 can be synthesized. Next, the Group 1 is introduced by a known method, whereby the compound of the present disclosure can be obtained.

The above Si(CD₃)₃₀H and (—Si(CD₃)₂O—)ₙ can be synthesized by a known method, and as an example, Examples 1 and 2 that will be described later can be referred to.

Another manufacturing method includes a method for manufacturing the compound of the present disclosure through a hydrogen-heavy hydrogen exchange reaction of a compound having the Group 1.

### [Composition]

The composition of the present disclosure may contain the compound of the present disclosure, and components other than the compound of the present disclosure are not particularly limited.

That is, the composition of the present disclosure may contain at least one type selected from the compound (1a), the compound (1b), the compound (1c), the compound (2a), the compound (2b), and the compound (2c). Note that the composition of the present disclosure may contain both of at least one type selected from the compound (1a), the compound (1b), and the compound (1c) and at least one type selected from the compound (2a), the compound (2b), and the compound (2c). The composition of the present disclosure may contain, for example, the compound (1a) and the compound (2a).

The composition of the present disclosure preferably contains the compound of the present disclosure and a liquid medium. When the composition of the present disclosure contains a liquid medium, the composition according to the present disclosure may be a solution, or may be a dispersion, as long as the composition is liquid.

The composition of the present disclosure may contain the compound of the present disclosure, and may contain impurities such as by-products generated in the step of manufacturing the compound of the present disclosure.

The content of the compound of the present disclosure is preferably from 0.001 to 40 mass %, more preferably from 0.01 to 20 mass %, and further preferably from 0.1 to 10 mass % based on the total amount of the composition of the present disclosure. In the case of the composition of the present disclosure used in the wet coating method, the content of the compound of the present disclosure may be from 0.01 to 10 mass %, from 0.02 to 5 mass %, from 0.03 to 3 mass %, or from 0.05 to 2 mass % based on the total amount of the composition of the present disclosure.

The liquid medium contained in the composition of the present disclosure may be of only one kind or two or more kinds.

The liquid medium is preferably an organic solvent.

Examples of organic solvents include compounds consisting solely of hydrogen atoms and carbon atoms, and compounds consisting solely of hydrogen atoms, carbon atoms, and oxygen atoms. Specifically, examples include hydrocarbon-based organic solvents, ketone-based organic solvents, ether-based organic solvents, ester-based organic solvents, glycol-based organic solvents, and alcohol-based organic solvents.

Specific examples of hydrocarbon-based organic solvents include pentane, hexane, heptane, octane, hexadecane, isohexane, isooctane, isononane, cycloheptane, cyclohexane, bicyclohexyl, benzene, toluene, ethylbenzene, o-xylene, m-xylene, p-xylene, o-diethylbenzene, m-diethylbenzene, p-diethylbenzene, n-butylbenzene, sec-butylbenzene, and tert-butylbenzene.

Specific examples of ketone-based organic solvents include acetone, methyl ethyl ketone, methyl isobutyl ketone, diisobutyl ketone, cyclohexanone, 2-heptanone, 4-heptanone, 3,5,5-trimethyl-2-cyclohexene-1-one, 3,3,5-trimethylcyclohexanone, and isophorone.

Specific examples of ether-based organic solvents include diethyl ether, cyclopentyl methyl ether, tetrahydrofuran, and 1,4-dioxane.

Specific examples of ester-based organic solvents include methyl acetate, ethyl acetate, propyl acetate, isopropyl acetate, butyl acetate, isobutyl acetate, tert-butyl acetate, amyl acetate, isoamyl acetate, ethyl 3-ethoxypropionate, ethyl lactate, ethylene glycol monobutyl ether acetate, diethylene glycol monobutyl ether acetate, propylene glycol monomethyl ether acetate, dipropylene glycol methyl ether acetate, 3-methoxy-3-methylbutyl acetate, 3-methoxybutyl acetate, propylene glycol monomethyl acetate, propylene glycol dimethyl acetate, ethylene glycol monoethyl ether acetate, ethylene glycol monomethyl ether acetate, diethylene glycol monoethyl ether acetate, cyclohexanol acetate, propylene glycol diacetate, propylene glycol monomethyl ether acetate, propylene glycol monomethyl ether propionate, propylene glycol monoethyl ether acetate, propylene glycol monobutyl ether acetate, propylene glycol monopropyl ether acetate, propylene glycol diacetate, 1,3-butylene glycol diacetate, 1,4-butanediol diacetate, 1,3-butylene glycol diacetate, 1,6-hexanediol diacetate, γ-butyrolactone, triacetin, and 2,2,4-trimethyl-1,3-pentanediol monoisobutyrate.

Specific examples of glycol-based organic solvents include ethylene glycol, ethylene glycol monobutyl ether, diethylene glycol monobutyl ether, triethylene glycol monobutyl ether, tetraethylene glycol monobutyl ether, ethylene glycol monohexyl ether, diethylene glycol monohexyl ether, ethylene glycol mono-2-ethylhexyl ether, diethylene glycol mono-2-ethylhexyl ether, propylene glycol monomethyl ether, propylene glycol monoethyl ether, propylene glycol monobutyl ether, propylene glycol monopropyl ether, ethylene glycol monoisopropyl ether, ethylene glycol monoethyl ether, ethylene glycol mono tert-butyl ether, ethylene glycol monopropyl ether, ethylene glycol monomethyl ether, diethylene glycol monoisopropyl ether, diethylene glycol monomethyl ether, dipropylene glycol monoethyl ether, dipropylene glycol monobutyl ether, dipropylene glycol monopropyl ether, dipropylene glycol monomethyl ether, tripropylene glycol monobutyl ether, tripropylene glycol monomethyl ether, propylene glycol monophenyl ether, 1,3-butylene glycol, propylene glycol n-propyl ether, propylene glycol n-butyl ether, diethylene glycol monoethyl ether, dipropylene glycol n-propyl ether, dipropylene glycol n-butyl ether, tripropylene glycol methyl ether, tripropylene glycol n-butyl ether, ethylene glycol dimethyl ether, ethylene glycol diethyl ether, ethylene glycol dibutyl ether, diethylene glycol diethyl ether, diethylene glycol dimethyl ether, dipropylene glycol dimethyl ether, diethylene glycol dibutyl ether, tetraethylene glycol dimethyl ether, dipropylene glycol dimethyl ether pentane, triethylene glycol dimethyl ether, and polyethylene glycol dimethyl ether.

Specific examples of alcohol-based organic solvents include methanol, ethanol, 1-propanol, isopropyl alcohol, n-butanol, diacetone alcohol, isobutanol, sec-butanol, tert-butanol, pentanol, 3-methyl-1,3-butanediol, 1,3-butanediol, 1,3-butylene glycol, octanediol, 2,4-diethylpentanediol, butylethylpropanediol, 2-methyl-1,3-propanediol, 4-hydroxy-4-methyl-2-pentanone, 2-ethyl-1-hexanol, 3,5,5-trimethyl-1-hexanol, isodecanol, isotridecanol, 3-methoxy-3-methyl-1-butanol, 2-methoxybutanol, 3-methoxybutanol, cyclohexanol, furfuryl alcohol, tetrahydrofurfuryl alcohol, benzyl alcohol, and methylcyclohexanol.

Further, examples of organic solvents include a halogen-based organic solvent, a nitrogen-containing compound, a sulfur-containing compound, a siloxane compound, and a fluorine-containing organic solvent.

Specific examples of halogen-based organic solvents include dichloromethane, chloroform, carbon tetrachloride, dichloroethane, chlorobenzene, o-chlorotoluene, m-chlorotoluene, p-chlorotoluene, m-dichlorobenzene, and 1,2,3-trichloropropane.

Examples of nitrogen-containing compounds include nitrobenzene, acetonitrile, benzonitrile, N,N-dimethylformamide, N,N-dimethylacetamide, N-methylpyrrolidone, and 1,3-dimethyl-2-imidazolidinone.

Examples of sulfur-containing compounds include carbon disulfide and dimethyl sulfoxide.

Examples of siloxane compounds include hexamethyldisiloxane, hexaethyldisiloxane, octamethyltrisiloxane, octaethyltrisiloxane, hexamethylcyclotrisiloxane, hexaethylcyclotrisiloxane, octamethylcyclotetrasiloxane, octaethylcyclotetrasiloxane, and decamethyltetrasiloxane.

Examples of fluorine-containing organic solvents include polyfluoro aromatic hydrocarbons (e.g., 1,3-bis(trifluoromethyl) benzene); polyfluoro aliphatic hydrocarbons (e.g., C₆F₁₃CH₂CH₃ (e.g., Asahikrin (Registered Trademark) AC-6000 manufactured by AGC Inc.,), 1,1,2,2,3,3,4-heptafluorocyclopentane (e.g., Zeorora (Registered Trademark) H manufactured by Zeon Corporation); hydrofluoroethers (HFE) (e.g., perfluoroalkyl ethers (perfluoroalkyl group and alkyl group may be linear chain or branched) such as perfluoropropyl methyl ether (C₃F₇OCH₃) (e.g., Novec (Trademark) 7000 manufactured by 3M Japan Limited), perfluorobutyl methyl ether (C₄F₉OCH₃) (e.g., Novec (Trademark) 7100 manufactured by 3M Japan Limited), perfluorobutyl ethyl ether (C₄F₉OC₂H₅) (e.g., Novec (Trademark) 7200 manufactured by 3M Japan Limited), and perfluorohexyl methyl ether (C₂F₅CF(OCH₃)C₃F₇) (e.g., Novec (Trademark) 7300 manufactured by 3M Japan Limited), and CF₃CH₂OCF₂CHF₂ (e.g., Asahikrin (Registered Trademark) AE-3000 manufactured by AGC Inc.,); and hydrofluoroolefins (HFO) (e.g., 1-chloro-2,3,3-trifluoro-1-propene (HCFO-1233yd) (e.g., Amolea (Registered Trademark) AS-300 manufactured by AGC Inc.,)).

The content of the liquid medium is preferably from 60 to 99.999 wt. %, more preferably from 80 to 99.99 wt. %, and still more preferably from 90 to 99.9 wt. % based on the total amount of the composition according to the present disclosure. In the case where the composition according to the present disclosure is used in a wet coating method, the content of the liquid medium may be from 90 to 99.99 wt. %, from 95 to 99.98 wt. %, from 97 to 99.97 wt. %, or from 98 to 99.95 wt. % based on the total amount of the composition according to the present disclosure.

The composition of the present disclosure may contain other components in addition to the compound of the present disclosure and the liquid medium as long as the effects of the present disclosure are not impaired.

Examples of other components include known additives such as acid catalysts and basic catalysts that promote hydrolysis and condensation reaction of reactive silyl groups.

An arbitrary suitable acid or base, a transition metal (e.g., Ti, Ni, Sn, Zr, Al or B), a sulfur-containing compound having a non-covalent electron pair in the molecular structure, a nitrogen-containing compound (e.g., a sulfoxide compound, an aliphatic amine compound, an aromatic amine compound, a phosphoric acid amide compound, an amide compound, and a urea compound) can be used as the catalyst.

Examples of acid catalysts include acetic acid, formic acid, trifluoroacetic acid, hydrochloric acid, nitric acid, sulfuric acid, phosphoric acid, sulfonic acid, methanesulfonic acid, and p-toluenesulfonic acid.

Further, examples of base catalysts include ammonia, sodium hydroxide, and potassium hydroxide; and organic amines such as triethylamine and diethylamine.

Further, examples of other components also include metal compounds having hydrolyzable groups (metal compounds having hydrolyzable groups are hereinafter also referred to as “specific metal compounds”). When the composition according to the present disclosure contains a specific metal compound, the sliding property and the antifouling property of the surface treatment layer can be further improved. Examples of specific metal compounds include a metal compound represented by one of Formulas (M1) to (M3).

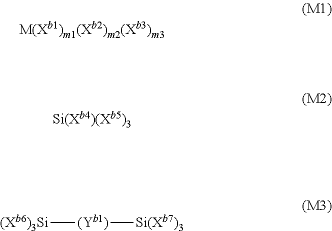

In Formula (M1),
M represents a trivalent or tetravelent metal atom.
X^{b1} each independently represents a hydrolyzable group.
X^{b2} each independently represents a siloxane skeleton-containing group.
X^{b3} each independently represents a hydrocarbon chain-containing group.
m1 is an integer from 2 to 4,
m2 and m3 are each independently an integer from 0 to 2, and
when M is a trivalent metal atom, m1+m2+m3 is 3, whereas when M is a tetravelent metal atom, m1+m2+m3 is 4.

In Formula (M2),
X^{b4} represents a hydrolyzable silane oligomer group, and
X^{b5} each independently represents a hydrolyzable group or an alkyl group having a number of carbon atoms from 1 to 4.

In Formula (M3),
X^{b6} and X^{b7} each independently represent a hydrolyzable group or a hydroxyl group, and
Y^{b1} represents a divalent organic group.

The metal represented by M in Formula (M1) includes semimetals such as Si and Ge. M is preferably a trivalent metal and a tetravalent metal, more preferably Al, Fe, In, Hf, Si, Ti, Sn, and Zr, further preferably Al, Si, Ti, and Zr, and particularly preferably Si.

Specific examples of the hydrolyzable group represented by X^{b1} in Formula (M1) include the same group as the hydrolyzable group represented by L in [—Si(R¹)ₙL₃₋ₙ] in the reactive silyl group.

The siloxane skeleton-containing group represented by X^{b2} has a siloxane unit (—Si—O—), and may be either linear or branched. The siloxane unit is preferably a dialkylsilyloxy group, and examples include a dimethylsilyloxy group and a diethylsilyloxy group. The number of repetitions of the siloxane unit in the siloxane skeleton-containing group is 1 or more, preferably from 1 to 5, more preferably from 1 to 4, and further preferably from 1 to 3.

The siloxane skeleton-containing group may contain a divalent hydrocarbon group in a part of the siloxane skeleton. Specifically, an oxygen atom in a part of the siloxane skeleton may be replaced with a divalent hydrocarbon group. Examples of the aforementioned divalent hydrocarbon group include alkylene groups such as a methylene group, an ethylene group, a propylene group, and a butylene group.

A hydrolyzable group, a hydrocarbon group (preferably an alkyl group), or the like may be bonded to a silicon atom at the end of the siloxane skeleton-containing group.

The number of elements of the siloxane skeleton-containing group is preferably 100 or less, more preferably 50 or less, and further preferably 30 or less. The number of elements is preferably 10 or more.

The siloxane skeleton-containing group is preferably a group represented by *—(O—Si(CH₃)₂)ₙCH₃, where n is an integer from 1 to 5, and * represents a part bonded to an adjacent atom.

The hydrocarbon chain-containing group represented by X^{b3} may be a group consisting solely of a hydrocarbon chain or a group having an etheric oxygen atom between carbon atoms of a hydrocarbon chain. The hydrocarbon chain may be linear or branched, and is preferably linear. The hydrocarbon chain may be a saturated hydrocarbon chain or an unsaturated hydrocarbon chain, and is preferably a saturated hydrocarbon chain. The number of carbon atoms of the hydrocarbon chain-containing group is preferably from 1 to 3, more preferably from 1 to 2, and further preferably 1. The hydrocarbon chain-containing group is preferably an alkyl group, and more preferably a methyl group, an ethyl group, or a propyl group.

m1 is preferably 3 or 4.

The compound represented by Formula (M1) is preferably a compound represented by the following Formulas (M1-1) to (M1-5) in which M is Si, and more preferably a compound represented by Formula (M1-1). The compound represented by Formula (M1-1) is preferably tetraethoxysilane, tetramethoxysilane, or triethoxymethylsilane.

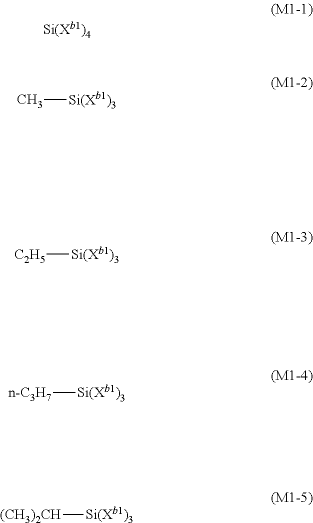

In Formula (M2), the number of silicon atoms contained in the hydrolyzable silane oligomer group represented by X^{b4} is preferably 3 or more, more preferably 5 or more, and further preferably 7 or more. The number of silicon atoms is preferably 15 or less, more preferably 13 or less, and further preferably 10 or less.

The hydrolyzable silane oligomer group may have an alkoxy group that is bonded to a silicon atom. Examples of the alkoxy group include a methoxy group, an ethoxy group, a propoxy group, a butoxy group, and the like, and preferably a methoxy group and an ethoxy group. The hydrolyzable silane oligomer group may have one or two or more types of alkoxy groups, and preferably has one type of alkoxy group.

Examples of the hydrolyzable silane oligomer group include (C₂H₅O)₃Si—(OSi(OC₂H₅)₂)₄O—*, and the like. Note that * represents a part bonded to an adjacent atom.

Examples of the hydrolyzable group represented by X^{b5} in Formula (M2) include the same group as the hydrolyzable group represented by L in [—Si(R′)ₙL₃₋ₙ] in the reactive silyl group, a cyano group, a hydrogen atom, and an allyl group, and preferably an alkoxy group or an isocyanato group. The alkoxy group is preferably an alkoxy group having a number of carbon atoms from 1 to 4.

X^{b5} is preferably a hydrolyzable group.

Examples of the compound represented by Formula (M2) include (H₅C₂O)₃—Si—(OSi(OC₂H₅)₂)₄OC₂H₅.

The compound represented by Formula (M3) is a compound having a reactive silyl group at both terminals of a divalent organic group, that is, bissilane.

Examples of the hydrolyzable group represented by X^{b6} and X^{b7} in Formula (M3) include an alkoxy group, an acyloxy group, a ketoxime group, an alkenyloxy group, an amino group, an aminoxy group, an amide group, an isocyanato group, and a halogen atom, and preferably an alkoxy group and an isocyanato group. As the alkoxy group, an alkoxy group having a number of carbon atoms from 1 to 4 is preferable, and a methoxy group and an ethoxy group are more preferable.

In Formula (M3), X^{b6} and X^{b7} may be the same group or different groups from each other. From the viewpoint of availability, X^{b6} and X^{b7} are preferably the same group.

In Formula (M3), Y^{b1} is a divalent organic group linking reactive silyl groups at both terminals. The number of carbon atoms in Y^{b1} of the divalent organic group is preferably from 1 to 8, and more preferably from 1 to 3.

Examples of Y^{b1} include an alkylene group, a phenylene group, and an alkylene group having an etheric oxygen atom between carbon atoms. Examples thereof include —CH₂CH₂—, —CH₂CH₂CH₂—, —CH₂CH₂CH₂CH₂—, —CH₂CH₂CH₂CH₂CH₂—, —CH₂CH₂CH₂CH₂CH₂CH₂—, —CH₂C(CH₃)₂CH₂—, —C(CH₃)₂CH₂CH₂C(CH₃)₂—, —CH₂CH₂OCH₂CH₂—, —CH₂CH₂CH₂OCH₂CH₂CH₂—, —CH(CH₃)CH₂OCH₂CH(CH₃)—, and —C₆H₄—.

Examples of the compound represented by Formula (M3) include (CH₃O)₃Si(CH₂)₂Si(OCH₃)₃, (C₂H₅O)₃Si(CH₂)₂Si(OC₂H₅)₃, (OCN)₃Si(CH₂)₂Si(NCO)₃, Cl₃Si(CH₂)₂SiCl₃, (CH₃O)₃Si(CH₂)₆Si(OCH₃)₃, and (C₂H₅O)₃Si(CH₂)₆Si(OC₂H₅)₃.

The content of other components that may be contained in the composition of the present disclosure is preferably 10 mass % or less and more preferably 1 mass % or less based on the total amount of the composition according to the present disclosure. When the composition according to the present disclosure contains a specific metal compound, the content of the specific metal compound is preferably from 0.01 to 30 mass %, more preferably from 0.01 to 10 mass %, and further preferably from 0.05 to 5 mass % based on the total amount of the composition of the present disclosure.

The total content (hereinafter also referred to as “solid content concentration”) of the compound of the present disclosure and the other components is preferably from 0.001 to 40 wt. %, more preferably from 0.01 to 20 wt. %, and still more preferably from 0.1 to 10 wt. % based on the total amount of the composition according to the present disclosure. The solid content concentration of the composition according to the present disclosure is a value calculated from the mass of the composition before being heated and the mass thereof after being heated in a convection-type dryer at 120° C. for 4 hours.

Since the composition according to the present disclosure contains a liquid medium, it is useful as a coating application and can be used as a coating liquid.

Examples of other components include compounds represented by the following Formula (5):

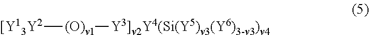

where in Formula 5, Y² is Si, Sn, or Ge,
Y¹ is each independently a hydrocarbon group or a trialkylsilyloxy group,
v1 is 0 or 1,
Y³ is each independently an alkylene chain or a polyalkylene oxide chain, or a combination of an alkylene chain with a divalent polysiloxane residue,
Y⁴ is a single bond or a (v2+v4)-valent linking group,
Y⁵ is each independently a hydrocarbon group,
Y⁶ is each independently a hydrolyzable group or a hydroxyl group,
v3 is each independently an integer from 0 to 2, and
v2 and v4 are each independently an integer of 1 or more.

Y³ is preferably an alkylene chain or a polyalkylene oxide chain.

Specific examples of the compound (5) include the compounds shown below. γ in the formula is preferably from 9 to 50, more preferably from 11 to 30, and particularly preferably from 11 to 25.

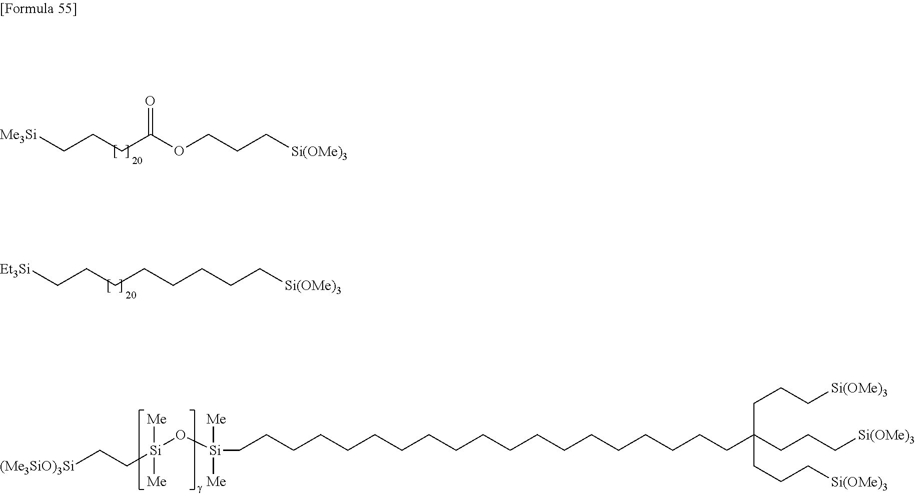

When the other component in the surface treatment agent disclosed herein is the compound (5), the content of the compound (5) is preferably 50 mass % or less, and more preferably 40 mass % or less.

### [Surface Treatment Agent]

In an embodiment, a surface treatment agent according to the present disclosure contains a compound according to the present disclosure. That is, the surface treatment agent according to the present disclosure may contain at least one type selected from the compound (1a), the compound (1b), the compound (1c), the compound (2a), the compound (2b), and the compound (2c). A surface treatment agent according to the present disclosure may contain both of one type selected from the compound (1a), the compound (1b), and the compound (1c) and one type selected from the compound (2a), the compound (2b), and the compound (2c), and may contain, for example, the compound (1a) and the compound (2a).

Further, the surface treatment agent according to the present disclosure may contain a compound according to the present disclosure and a liquid medium. The surface treatment agent according to the present disclosure may be a composition of the present disclosure. Preferable forms of the liquid medium included in the surface treatment agent are the same as preferable forms of the liquid medium included in the composition of the present disclosure.

The compound according to the present disclosure includes an alkyl group having a number of carbon atoms of 2 or more, an organopolysiloxane group, and a reactive silyl group. Therefore, by using a surface treatment agent comprising the compound of the present disclosure, a surface treatment layer with excellent water repellency and abrasion resistance can be formed.

The surface treatment agent according to the present disclosure is preferably used, in particular, for optical members.

### [Article]

In an embodiment, an article according to the present disclosure includes a substrate and a surface treatment layer which is disposed on the substrate and of which the surface is treated with a surface treatment agent according to the present disclosure.

The surface treatment layer may be formed on a part of the surface of the substrate or over the entire surface of the substrate. The surface treatment layer may be spread in the form of a film on the surface of the substrate or scattered in the form of dots.

In the surface treatment layer, the compound of the present disclosure is contained therein in a state in which the hydrolysis of some or all of reactive silyl groups have progressed and the dehydration condensation reaction of silanol groups have progressed.

The thickness of the surface treatment layer is preferably from 1 to 100 nm and more preferably from 1 to 50 nm. When the thickness of the surface treatment layer is 1 nm or more, the effects of the surface treatment are likely to be obtained satisfactorily. When the thickness of the surface treatment layer is 100 nm or smaller, the usage efficiency is high. The thickness of the surface treatment layer can be calculated from the oscillation cycle of the interference pattern of the reflected X-ray, which is obtained by an X-ray reflectivity method using an X-ray diffractometer for thin film analysis (Product name: ATX-G, manufactured by Rigaku Corporation).

The type of the substrate is not limited to any particular types, and examples include a substrate which is required to have water repellency. Examples of the substrate include a substrate which may be used while touching it with another article (e.g., a stylus) or a human hand or fingers; a substrate which may be held by a human hand or fingers during the operation; and a substrate which may be placed on another article (e.g., a mounting table).

Examples of the material of the substrate include metals, resins, glass, sapphire, ceramics, semiconductors, stones, fibers, nonwoven fabric, paper, wood, fur, natural leather, artificial leather, pottery, and composite materials thereof. The glass may be one that is chemically reinforced.

Examples of substrates include building materials, decorative building materials, interior goods, transportation apparatuses (e.g., automobiles), signboards, bulletin boards, drinking containers, tableware, water tanks, ornamental apparatuses (e.g., frames and boxes), laboratory apparatuses, furniture, textile products, and packaging containers; glass or resins used for art, sports, games and the like; glass or resins used for exterior parts (excluding display parts) of apparatuses such as mobile phones (e.g., smartphones), mobile information terminals, game machines, and remote controllers. The shape of the substrate may be plate-like or film-like.

The substrate is preferably a substrate for a touch panel, a substrate for a display, or a lens for eyeglasses, and particularly preferably a substrate for a touch panel. The material of a substrate for a touch panel is preferably glass or a transparent resin.

The substrate may be a substrate of which a surface treatment such as a corona discharge treatment, a plasma treatment, a plasma graft polymerization treatment, or the like has been performed on one or both of the surfaces. The substrate subjected to the surface treatment has a more excellent adhesive property for the surface treatment layer, and the abrasion resistance of the surface treatment layer is further improved. Therefore, it is preferred to perform a surface treatment on the surface of the substrate on the side which is brought into contact with the surface treatment layer. Further, in the case where an underlayer (which will be described later) is provided, the substrate subjected to the surface treatment has a more excellent adhesive property for the underlayer, and the abrasion resistance of the surface treatment layer is further improved. Therefore, in the case where an underlayer is provided, it is preferred to perform a surface treatment on the surface of the substrate on the side which is brought into contact with the underlayer.

The surface treatment layer may be directly disposed on the surface of the substrate, or an underlayer may be provided between the substrate and the surface treatment layer. In order to further improve the water repellency and the abrasion resistance of the surface treatment layer, an article according to the present disclosure preferably includes a substrate, an underlayer disposed on the substrate, and a surface treatment layer which is disposed on the underlayer and of which the surface is treated with a surface treatment agent according to the present disclosure.

The underlayer is preferably a layer containing an oxide containing silicon and at least one specific element selected from the group consisting of Group 1 elements, Group 2 elements, Group 4 elements, Group 5 elements, Group 13 elements, and Group 15 elements in the periodic table.

Group 1 elements in the periodic table (hereinafter also referred to as “Group 1 elements”) mean lithium, sodium, potassium, rubidium, and cesium. Group 1 elements are preferably lithium, sodium, and potassium, and more preferably sodium and potassium because the surface treatment layer can be formed more uniformly on the underlayer without defects or because variations in the composition of the underlayer among samples are more suppressed. The underlayer may contain two or more kinds of Group 1 elements.

Group 2 elements in the periodic table (hereinafter also referred to as “Group 2 elements”) mean beryllium, magnesium, calcium, strontium, and barium. Group 2 elements are preferably magnesium, calcium, and barium, and more preferably magnesium and calcium because the surface treatment layer can be formed more uniformly on the underlayer without defects or because variations in the composition of the underlayer among samples are more suppressed. The underlayer may contain two or more kinds of Group 2 elements.

Group 4 elements in the periodic table (hereinafter also referred to as “Group 4 elements”) mean titanium, zirconium, and hafnium. Group 4 elements are preferably titanium and zirconium, and more preferably titanium because the surface treatment layer can be formed more uniformly on the underlayer without defects or because variations in the composition of the underlayer among samples are more suppressed. The underlayer may contain two or more kinds of Group 4 elements.

Group 5 elements in the periodic table (hereinafter also referred to as “Group 5 elements”) mean vanadium, niobium, and tantalum. Group 5 elements are particularly preferably vanadium because the abrasion resistance of the surface treatment layer becomes more excellent. The underlayer may contain two or more kinds of Group 5 elements.

Group 13 elements in the periodic table (hereinafter also referred to as “Group 13 elements”) mean boron, aluminum, gallium, and indium. Group 13 elements are preferably boron, aluminum, and gallium, and more preferably boron and aluminum because the surface treatment layer can be formed more uniformly on the underlayer without defects or because variations in the composition of the underlayer among samples are more suppressed. The underlayer may contain two or more kinds of Group 13 elements.

Group 15 elements in the periodic table (hereinafter also referred to as “Group 15 elements”) mean nitrogen, phosphorus, arsenic, antimony, and bismuth. Group 15 elements are preferably phosphorus, antimony, and bismuth, and more preferably phosphorus and bismuth because the surface treatment layer can be formed more uniformly on the underlayer without defects or because variations in the composition of the underlayer among samples are more suppressed. The underlayer may contain two or more kinds of Group 15 elements.

The specific element contained in the underlayer is preferably a Group 1 element, a Group 2 element, and a Group 13 element, more preferably a Group 1 element and a Group 2 element, and still more preferably a Group 1 element because the abrasion resistance of the surface treatment layer becomes more excellent.

Only one kind of element or two or more kinds of elements may be contained as the specific element(s).

The oxide contained in the underlayer may be a mixture of oxides each comprising only one of the aforementioned elements (silicon and specific element) (e.g., a mixture of silicon oxide and an oxide of a specific element), a composite oxide comprising two or more kinds of the aforementioned elements, or a mixture of an oxide comprising only one of the aforementioned elements and a composite oxide.

The ratio of the total molarity of the specific element in the underlayer to the molarity of silicon in the underlayer (specific element/silicon) is preferably from 0.02 to 2.90, more preferably from 0.10 to 2.00, and still more preferably from 0.20 to 1.80 because the abrasion resistance of the surface treatment layer becomes more excellent.

The molarity (mol %) of each element in the underlayer can be measured, for example, by a depth-direction analysis by X-ray photoelectron spectroscopy (XPS) using ion sputtering.

The underlayer may be a single layer or may consist of a plurality of layer. The underlayer may have an uneven surface(s).

The thickness of the underlayer is preferably from 1 to 100 nm, more preferably from 1 to 50 nm, and still more preferably from 2 to 20 nm. When the thickness of the underlayer is equal to or larger than the aforementioned lower limit value, the adhesive property of the surface treatment layer by the underlayer is further improved, so that the abrasion resistance of the surface treatment layer becomes more excellent. When the thickness of the underlayer is equal to or less than the aforementioned upper limit value, the abrasion resistance of the underlayer itself becomes excellent.

The thickness of the underlayer is measured by observing the cross section of the underlayer by a transmission electron microscope (TEM).

The underlayer can be formed, for example, by a vapor-deposition method using a vapor-deposition material or a wet coating method.

The vapor-deposition material used in the vapor-deposition method preferably an oxide comprising silicon and a specific element.

Specific examples of the form of the vapor-deposition material include a powder, a molten material, a sintered compact, granules, and a crushed material. Further, a molten material, a sintered compact, and granules are preferred in view of the handling property.

Note that the molten material refer to a solid material obtained by melting a powder of a vapor-deposition material at a high temperature, and then cooling and solidifying the molten vapor-deposition material. The sintered compact refers to a solid material obtained by firing a powder of a vapor-deposition material. Further, if necessary, instead of the powder of the vapor-deposition material, a molded compact formed by pressing the powder may be used. The granules refers to a solid material obtained by mixing and kneading a powder of a vapor-deposition material and a liquid medium (e.g., water or an organic solvent) and thereby obtaining particles thereof, and then drying the obtained particles.

The vapor-deposition material can be manufactured, for example, by the following methods.
A method in which a powder of a vapor-deposition material is obtained by mixing a powder of silicon oxide with a powder of an oxide of a specific element.
A method in which granules of a vapor-deposition material is obtained by, after obtaining particles by mixing and kneading a powder of the aforementioned vapor-deposition material and water, drying the obtained particles.
A method in which a sintered compact is obtained by drying a mixture obtained by mixing a powder comprising silicon (e.g., a powder made of silicon oxide, silica sand, or silica gel), a powder comprising a specific element (e.g., a powder of an oxide of a specific element, a carbonate, a sulfate, a nitrate, an oxalate, or a hydroxide), and water, and then firing the dried mixture or a molded article obtained by press-molding the dried mixture.
A method in which a molten material is obtained by melting a powder comprising silicon (e.g., a powder made of silicon oxide, silica sand, or silica gel) and a powder comprising a specific element (e.g., a powder of an oxide of a specific element, a carbonate, a sulfate, a nitrate, an oxalate, or a hydroxide) at a high temperature, and then cooling and solidifying the molten substance.

Specific examples of the vapor-deposition method using a vapor-deposition material include a vacuum vapor-deposition method. The vacuum vapor-deposition method is a method in which a vapor-deposition material is evaporated in a vacuum chamber and deposited on the surface of a substrate.

The temperature during the vapor-deposition (e.g., in the case where a vacuum deposition apparatus is used, the temperature of a boat in which the deposition material is disposed) is preferably from 100 to 3,000° C. and more preferably from 500 to 3,000° C.

The pressure during the vapor-deposition (e.g., in the case where a vacuum deposition apparatus is used, the pressure in a chamber in which the deposition material is disposed) is preferably 1 Pa or lower, and more preferably 0.1 Pa or lower.

When an underlayer is formed by using a vapor-deposition material, one vapor-deposition material may be used, or two or more vapor-deposition materials comprising different elements may be used.

Specific examples of the method for evaporating a vapor-deposition material include a resistive heating method in which a vapor-deposition material is melted and evaporated on a resistive heating boat made of a high melting-point metal, and an electron gun method in which a vapor-deposition material is irradiated with an electron beam and thereby directly heated, so that its surface is melted and evaporated. As the method for evaporating a vapor-deposition material, the electron gun method is preferred because since the material can be locally heated, even a material having a high melting point can be evaporated, and since an area that is not irradiated with the electron beam is kept at a low temperature, there is no risk of reaction with the container nor risk of contamination by impurities.

As the method for evaporating a vapor-deposition material, a plurality of boats may be used, or the whole vapor-deposition materials may be put in a single boat. The vapor-deposition method may be co-vapor deposition or alternating vapor deposition. Specific examples include an example in which silica and a specific source are mixed and used in the same boat, an example in which co-vapor deposition is performed in a state in which silica and a specific element source are put in separate boats, and an example in which alternating vapor deposition is performed in a similar manner, i.e., in a state in which silica and a specific element source are put in separate boats. The conditions for the vapor deposition, the order, and the like are selected as appropriate according to the structure of the underlayer.

In the wet coating method, it is preferred to form an underlayer on a substrate by a wet coating method using a coating liquid comprising a compound comprising silicon, a compound comprising a specific element, and a liquid medium.

Specific examples of silicon compounds include silicon oxide, silicic acid, partial condensate of silicic acid, alkoxysilane, and partial hydrolysis condensate of alkoxysilane.

Specific examples of compounds comprising a specific element include oxides of the specific element, alkoxides of the specific element, carbonates of the specific element, sulfates of the specific element, nitrates of the specific element, oxalates of the specific element, and hydroxides of the specific element.

Examples of liquid mediums include those similar to liquid mediums contained in the composition according to the present disclosure.

The content of the liquid medium is preferably from 0.01 to 20 mass % and more preferably from 0.1 to 10 mass % based on the total amount of the coating liquid used for the formation of the underlayer.

Specific examples of the wet coating method for forming an underlayer include a spin coating method, a wipe coating method, a spray coating method, a squeegee coating method, a dip coating method, a die coating method, an ink jet method, a flow coating method, a roll coating method, a casting method, a Langmuir-Blodgett method, and a gravure coating method.

After the substrate or the like is wet-coated with the coating liquid, the coating is preferably dried. The drying temperature of the coating is preferably from 20 to 200° C. and more preferably from 80 to 160° C.

The article according to the present disclosure may be an optical member including a surface treatment layer as the outermost layer.

Examples of preferred optical members include a wide variety of optical members in addition to optical members related to displays or the like.

Examples of optical members include a cathode ray tube (CRT; e.g., a computer monitor), a display such as a liquid crystal display, a plasma display, an organic EL display, an inorganic thin film EL dot matrix display, a rear projection-type display, a vacuum fluorescent display (VFD), and a field emission display (FED), a protective plate for such a display, or those in which an antireflection film treatment is performed on their surfaces.

The article according to the present disclosure is preferably an optical member. Examples of optical members include a car navigation system, a mobile phone, a smartphone, a digital camera, a digital video camera, a PDA, a portable audio player, a car audio system, a game apparatus, a lens for eyeglasses, a camera lens, a lens filter, sunglasses, a medical apparatus such as a gastroscope, a copying machine, a PC, a display (e.g., a liquid crystal display, an organic EL display, a plasma display, and a touch panel display), a touch panel, a protective film, and an antireflection film.

Further, examples of optical members include a front protective plate for a display such as a PDP and an LCD, an antireflection plate, a polarizing plate, and an antiglare plate; a disk surface of an optical disk such as a Blu-ray (Blu-ray (Registered Trademark)) disk, a DVD disk, a CD-R, and an MO; an optical fiber; and a display surface of a clock or a watch.

In particular, the article according to the present disclosure is preferably a display or a touch panel.

The article according to the present disclosure may be a medical apparatus or a medical material. Further, the article according to the present disclosure may also be an automobile interior or exterior member. Examples of exterior members include a window, a light cover, and an external camera cover. Examples of interior members include an instrument panel cover, a navigation system touch panel, and a decorative interior member.

When the article according to the present disclosure is an optical member, the material constituting the surface of the substrate is a material for an optical member, e.g., glass or transparent plastic. Further, when the article according to the present disclosure is an optical member, a functional layer such as a hard coat layer or an antireflection layer may be formed on the surface (outermost layer) of the substrate. The antireflection layer may be either a single-layer antireflection layer or a multi-layer antireflection layer.

Examples of inorganic substances that can be used for the antireflection layer include SiO₂, SiO, ZrO₂, TiO₂, TiO, Ti₂O₃, Ti₂O₅, Al₂O₃, Ta₂O₅, Ta₃O₅, Nb₂O₅, HfO₂, Si₃N₄, CeO₂, MgO, Y₂O₃, SnO₂, MgF₂, and WO₃. These inorganic substances may be used alone or in combination (e.g., as a mixture) of two or more of them. In the case of the multi-layer antireflection layer, it is preferred to use SiO₂ and/or SiO in the outermost layer. When the article according to the present disclosure is an optical glass component for a touch panel, a thin film using a transparent electrode, e.g., indium tin oxide (ITO), indium zinc oxide, or the like, may be provided on a part of the surface of the substrate (glass). Further, the substrate may also include an insulating layer, an adhesive layer, a protective layer, a decorative frame layer (I-CON), an atomization film layer, a hard coating film layer, a polarizing film, a phase difference film, a liquid crystal display module, or the like according to its specific specifications.

### [Method for Manufacturing Article]

A method for manufacturing an article according to the present disclosure is, for example, a method for manufacturing an article including a surface treatment layer formed on a substrate by performing a surface treatment on the substrate by using a surface treatment agent according to the present disclosure. Examples of surface treatments include a dry coating method and a wet coating method.

Examples of dry coating methods include techniques such as vacuum vapor deposition, CVD, and sputtering. As the dry coating method, a vacuum vapor-deposition method is preferred in order to suppress the decomposition of the compound and in view of the simplicity of the apparatus. During the vacuum deposition, a pellet-like substance obtained by impregnating a porous material made of a metal such as iron or steel with a compound of the present disclosure may be used. A pellet-like substance, which is obtained by impregnating a porous material made of a metal such as iron or steel with a composition comprising a compound of the present disclosure and a liquid medium, and drying the liquid medium, may be used.

Examples of wet coating methods include a spin coating method, a wipe coating method, a spray coating method, a squeegee coating method, a dip coating method, a die coating method, an ink jet method, a flow coating method, a roll coating method, a casting method, a Langmuir-Blodgett method, and a gravure coating method.

In order to improve the abrasion resistance of the surface treatment layer, when necessary, an operation for accelerating the reaction between the compound of the present disclosure and the substrate may be performed. Examples of such operations include heating, humidification, and irradiation with light.

For example, it is possible to accelerate, by heating a substrate on which a surface treatment layer is formed in an atmosphere comprising moisture, the reaction such as a hydrolysis reaction of a hydrolyzable group, a reaction between a hydroxyl group or the like and a silanol group on the surface of the substrate, and formation of a siloxane bond by a condensation reaction of a silanol group.

After the surface treatment, compounds which are contained in the surface treatment layer and are not chemically bonded to other compounds nor the substrate may be removed as required. Examples of removal methods include a method in which a solvent is poured over the surface treatment layer, and a method in which the surface treatment layer or the like is wiped with a cloth impregnated with a solvent.

### EXAMPLES

The present invention will be described hereinafter in a more detailed manner by using Examples, but the present invention is not limited to these Examples. Note that, of Examples 1 to 9, Examples 1 to 8 are examples according to the present disclosure, and Example 9 is a comparative example.

### <Compound I-1>

2,3,4,5,6-pentafluorophenol (6.8 g), THF (tetrahydrofuran, 42 mL), and triethylamine (5.1 g) were added to a 100 mL flask under a nitrogen atmosphere, and the mixture was stirred at 25° C. Subsequently, 10-undecenoyl chloride (5.0 g) added dropwise to the reaction mixture, and the mixture was stirred for 1 hour at 25° C. 7.9 g of a compound I-1 was obtained by filtering the reaction solution, removing the solvent and low-boiling components by distillation under a reduced pressure, and performing flash column chromatography (developing solvent: hexane/ethyl acetate) using silica gel.

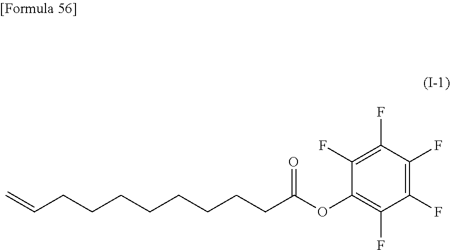 [SMILES: C=CCCCCCCCCC(=O)Oc1c(F)c(F)c(F)c(F)c1F]

### <Compound I-2>

The compound I-1 (5.6 g), THF (5.0 mL), chlorodimethylsilane (2.3 g), and a xylene solution of platinum/1,3-divinyl-1,1,3,3-tetramethyldisiloxane complex (platinum content: 3 mass %, 0.10 mL) were added to a 200 mL flask under a nitrogen atmosphere, and the mixture was stirred for 2 hours at 25° C. 7.2 g of a compound I-2 was obtained by removing the solvent and low-boiling components by distillation under a reduced pressure.

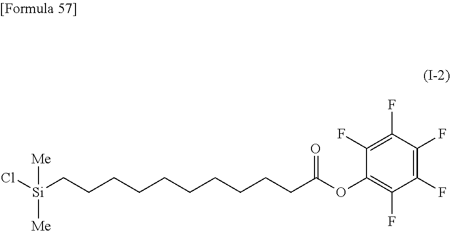 [SMILES: C[Si](C)(Cl)CCCCCCCCCCC(=O)Oc1c(F)c(F)c(F)c(F)c1F]

### <Compound I-3>

Diethyl ether (150 mL), ion-exchanged water (50 mL), and triethylamine (1.87 g) were added to a 500 mL flask, and the mixture was cooled with an ice bath. Next, trimethyl-d9-chlorosilane (2 g) was added dropwise while cooling the mixture with the ice bath, the temperature was raised to 25° C., and the mixture was stirred at 25° C. for 1 hour. Hexane (150 mL) was added to the reaction solution, the mixture was washed by ion-exchanged water and dehydrated by magnesium sulfate, and was removed by distillation under a reduced pressure to obtain 1.5 g of a compound I-3.

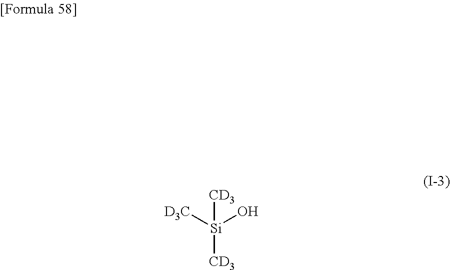 [SMILES: [2H]C([2H])([2H])[Si](O)(C([2H])([2H])[2H])C([2H])([2H])[2H]]

### <Compound I-4>

The compound I-3 (0.19 g) and THF (5 mL) were put in a 100 mL three-necked flask under a nitrogen atmosphere, and the mixture was stirred at 25° C. Next, the mixture was cooled to 0° C., and a hexane solution of n-BuLi (1.6 M, 0.9 mL) was added dropwise. Then, a THF solution of hexamethylcyclotrisiloxane (1.2 M, 1 mL) was added dropwise, and a THF solution of hexamethylcyclotrisiloxane (1.2 M, 9 mL) was added dropwise, and the mixture was stirred at 25° C. for 2 hours. After that, the compound I-2 (1.28 g) was added to the mixture, the mixture was stirred for 1 hour, 2,2-diallylpenta-4-ene-1-amine (0.95 g) was added, and the mixture was stirred at 25° C. for 1 hour. Then, hexane and ion-exchanged water were added to the reaction solution, and the organic layer was separated. 1.0 g of a compound I-4 was obtained by removing the solvent and low-boiling components by distillation under a reduced pressure and then performing flash column chromatography (developing solvent: hexane/ethyl acetate) using silica gel. The structure of the compound I-4 was confirmed from NMR data shown below. The average value of n in the compound I-4 was 18.

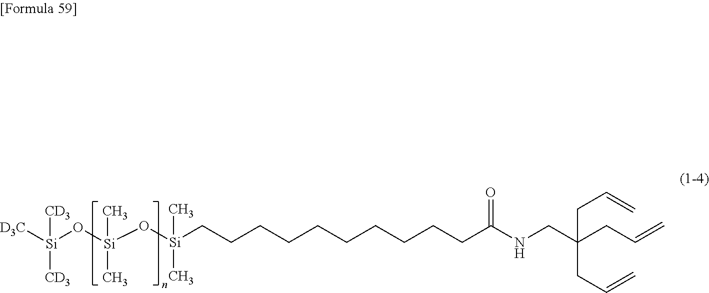

¹H-NMR (400 MHz, CDCl₃): δ=5.80 (ddt, J=17.7, 10.6, 7.4 Hz, 3H), 5.15-4.90 (m, 6H), 3.12 (d, J=6.3 Hz, 2H), 2.09 (t, J=7.5 Hz, 2H), 1.96 (dd, J=7.6, 1.3 Hz, 6H), 1.34-1.07 (m, 16H), 0.45 (t, J=7.6 Hz, 2H), 0.06-−0.10 (m, 114H).

### <Compound I-5>

Dichloromethane (10 g), the compound I-4 (0.80 g), a toluene solution of a platinum/1,3-divinyl-1,1,3,3-tetramethyldisiloxane complex (platinum content: 3 mass %, 4.1 mg), aniline (2.6 mg), and trimethoxysilane (0.40 g) were added to a 100 mL flask, and the mixture was stirred at 25° C. for 2 hours. 0.70 g of a compound I-5 was obtained by removing the solvent by distillation under a reduced pressure. The structure of the compound I-5 was confirmed from NMR data shown below. The average value of n in the compound I-5 was 18. As a result of the GPC measurement, the number average molecular weight (Mn) was 2,108.

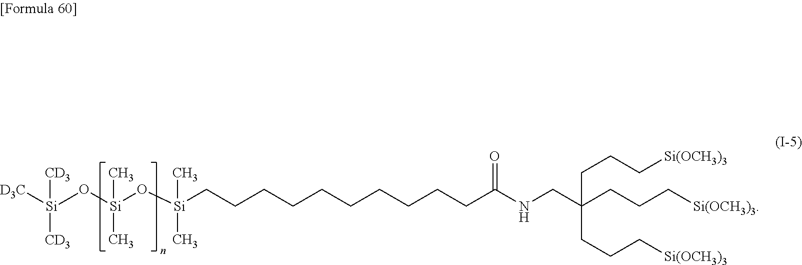

¹H-NMR (400 MHz, CDCl₃): δ=5.66 (d, J=6.4 Hz, 1H), 3.46 (s, 27H), 2.12 (t, J=7.5 Hz, 2H), 1.61-1.42 (m, 2H), 1.32-1.02 (m, 26H), 0.53 (t, J=7.7 Hz, 6H), 0.45 (t, J=7.0 Hz, 2H), 0.22-−0.24 (m, 114H).

### <Compound II-1>

A compound II-1 was obtained according to the method disclosed in Non-Patent Literature (Macromolecules 1982, 15, 1594).

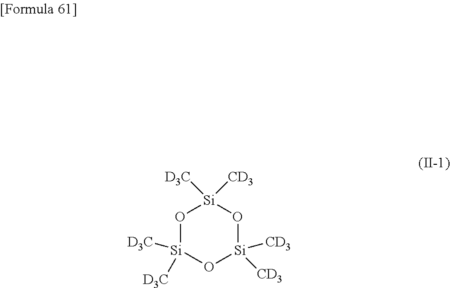 [SMILES: [2H]C([2H])([2H])[Si]1(C([2H])([2H])[2H])O[Si](C([2H])([2H])[2H])(C([2H])([2H])[2H])O[Si](C([2H])([2H])[2H])(C([2H])([2H])[2H])O1]

### <Compound II-2>

Trimethylsilanol (0.19 g) and THF (5 mL) were put in a 100 mL three-necked flask under a nitrogen atmosphere, and the mixture was stirred at 25° C. Next, the mixture was cooled to 0° C., and a hexane solution of n-BuLi (1.6 M, 0.9 mL) was added dropwise. Next, a THF solution of the compound II-1 (1.2 M, 1 mL) was added dropwise, a THF solution of the compound II-1 (1.2 M, 9 mL) was also added dropwise, and the mixture was stirred for 2 hours. After that, the compound I-1 (1.28 g) was added to the mixture, the mixture was stirred at 25° C. for 1 hour, then 2,2-diallylpenta-4-ene-1-amine (0.95 g) was added, and the mixture was stirred at 25° C. for 1 hour. Then, hexane and ion-exchanged water were added to the reaction solution, and the organic layer was separated. 1.1 g of a compound II-2 was obtained by removing the solvent and low-boiling components by distillation under a reduced pressure and then performing flash column chromatography (developing solvent: hexane/ethyl acetate) using silica gel. The structure of the compound II-2 was confirmed from NMR data shown below.

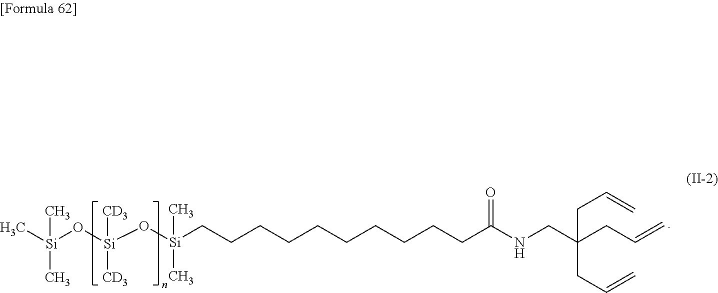

¹H-NMR (400 MHz, CDCl₃): δ=5.80 (ddt, J=17.7, 10.6, 7.4 Hz, 3H), 5.15-4.90 (m, 6H), 3.12 (d, J=6.3 Hz, 2H), 2.09 (t, J=7.5 Hz, 2H), 1.96 (dd, J=7.6, 1.3 Hz, 6H), 1.34-1.07 (m, 16H), 0.45 (t, J=7.6 Hz, 2H), 0.06-−0.10 (m, 15H).

### <Compound II-3>

Dichloromethane (10 g), the compound II-2 (0.80 g), a toluene solution of a platinum/1,3-divinyl-1,1,3,3-tetramethyldisiloxane complex (platinum content: 3 mass %, 4.1 mg), aniline (2.6 mg), and trimethoxysilane (0.40 g) were added to a 100 mL flask, and the mixture was stirred at 25° C. for 2 hours. 0.70 g of a compound II-3 was obtained by removing the solvent by distillation under a reduced pressure. The structure of the compound II-3 was confirmed from NMR data shown below.

As a result of the GPC measurement, the number average molecular weight (Mn) was 2,198.

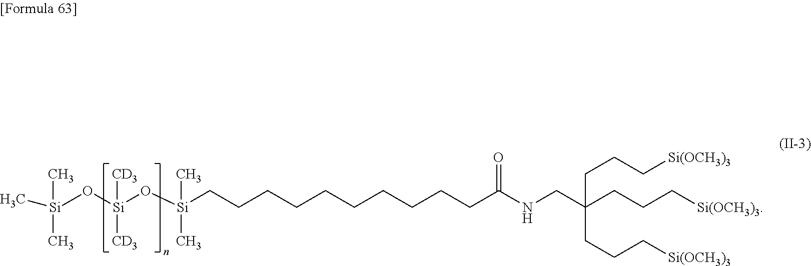

¹H-NMR (400 MHz, CDCl₃): δ=5.66 (d, J=6.4 Hz, 1H), 3.46 (s, 27H), 2.12 (t, J=7.5 Hz, 2H), 1.61-1.42 (m, 2H), 1.32-1.02 (m, 26H), 0.53 (t, J=7.7 Hz, 6H), 0.45 (t, J=7.0 Hz, 2H), 0.22-−0.24 (m, 15H).

### <Compound III-1>

A compound III-1 was synthesized according to Example 1 of International Patent Publication No. WO2023/017830.

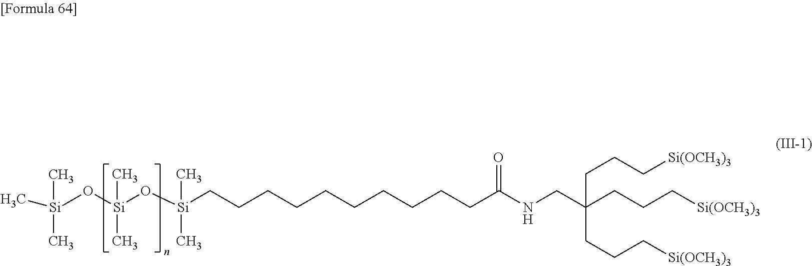

### <Compound IV-1>

18-bromo-1-octadecene (3 g), trichlorosilane (4 mL), and a toluene solution of a platinum/1,3-divinyl-1,1,3,3-tetramethyldisiloxane complex (platinum content: 3 mass %, 36 mg) were added to a 30 mL vial, and the mixture was stirred at 25° C. for 1 hour. After removing low-boiling components by distillation under a reduced pressure, THF, and a diethyl ether solution (1 M, 13 mL) of methyl-d3-magnesium iodide were added dropwise at 0° C., and the mixture was stirred for 3 hours. A mixed solution of triethylamine and water (1 mL/1 mL) was added, and the solution was stirred for 30 minutes. Hexane and a saturated salt solution were added to the reaction solution, and the organic layer was separated. 1.3 g of a compound IV-1 was obtained by removing the solvent and low-boiling components by distillation under a reduced pressure and performing flash column chromatography (developing solvent: hexane/ethyl acetate) using silica gel. The structure of the compound IV-1 was confirmed from NMR data shown below.

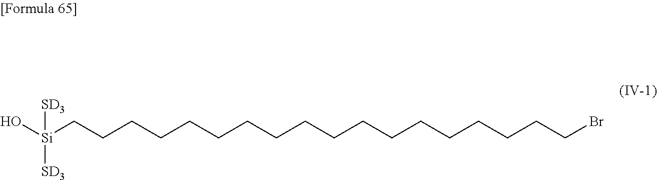

¹H-NMR (400 MHz, CDCl₃): δ=3.41 (t, J=6.9 Hz, 2H), 1.85 (m, 2H), 1.25 (s, 30H), 0.73-0.50 (m, 2H).

### <Compound IV-2>

A compound IV-2 was obtained according to the method according to Non-Patent Literature (Journal of the American Chemical Society 2016, 138, 4210).

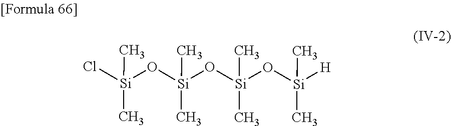 [SMILES: C[SiH](C)O[Si](C)(C)O[Si](C)(C)O[Si](C)(C)Cl]

### <Compound IV-3>

A compound IV-3 was obtained according to the method according to Non-Patent Literature (Polymer Chemistry 2020, 11, 7625).

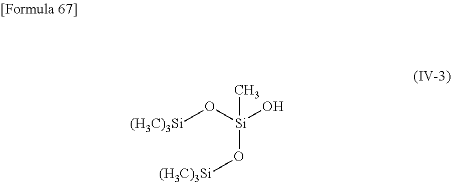

### <Compound IV-4>

The compound IV-2 (1.0 g), THF (10 mL), and triethylamine (0.9 mL) were added to a 30 mL vial, the compound IV-3 (1.34 g) was added dropwise, and the mixture was stirred at 25° C. for 2 hours. Hexane and ion-exchanged water were added to the mixture, and the organic layer was separated. 1.0 g of a compound IV-4 was obtained by removing the solvent and low-boiling components by distillation under a reduced pressure and then performing flash column chromatography (developing solvent: hexane/ethyl acetate) using silica gel. The structure of the compound IV-4 was confirmed from NMR data shown below.

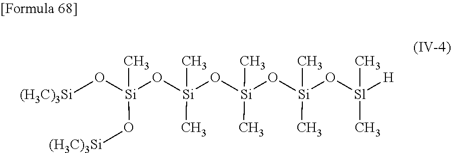

¹H-NMR (400 MHz, CDCl₃): δ=4.70 (sept, J=2.9 Hz, 1H), 0.26-−0.09 (m, 45H).

### <Compound IV-5>

A compound IV-4 (2 g), carbon tetrachloride (3.7 mL), and palladium chloride (0.007 g) were added to a 30 mL vial, and the mixture was stirred at 25° C. for 12 hours. 2.1 g of a compound IV-5 was obtained by filtering the reaction solution and removing the solvent and low-boiling components by distillation under a reduced pressure. The structure of the compound IV-5 was confirmed from NMR data shown below.

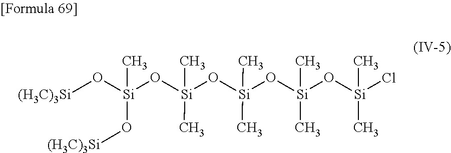

¹H-NMR (400 MHz, CDCl₃): δ=0.45 (s, 6H), 0.26-−0.09 (m, 39H).

### <Compound IV-6>

The compound IV-1 (0.4 g), dichloromethane (2 mL), and triethylamine (0.17 mL) were added to a 30 mL vial, the compound IV-(0.5 g) was added dropwise, and the mixture was stirred at 25° C. for 2 hours. Hexane and ion-exchanged water were added to the reaction solution, and the organic layer was separated. 0.5 g of a compound IV-6 was obtained by removing the solvent and low-boiling components by distillation under a reduced pressure and then performing flash column chromatography (developing solvent: hexane/ethyl acetate) using silica gel. The structure of the compound IV-6 was confirmed from NMR data shown below.

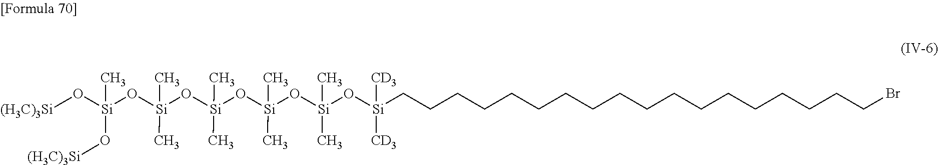

¹H-NMR (400 MHz, CDCl₃): δ=3.41 (t, J=6.9 Hz, 2H), 1.85 (dt, J=14.5, 7.0 Hz, 2H), 1.26 (d, J=4.1 Hz, 30H), 0.52 (m, 2H), 0.29-−0.27 (m, 45H).

### <Compound IV-7>

The compound IV-6 (0.5 g) and a THF solution of an Allyl Grignard reagent (2.0 M, 1.4 mL) were added under a nitrogen atmosphere, and the mixture was stirred at 0° C. for 12 hours. A saturated aqueous ammonium chloride solution and hexane were successively added to the reaction solution, and the organic layer was separated. 0.25 g of a compound IV-7 was obtained by removing the solvent and low-boiling components by distillation under a reduced pressure, performing flash column chromatography (developing solvent: hexane) using silica gel. The structure of the compound IV-7 was confirmed from NMR data shown below.

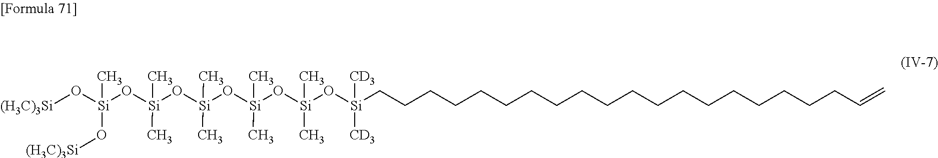

¹H-NMR (400 MHz, CDCl₃): δ=5.81 (ddt, J=17.0, 10.2, 6.7 Hz, 1H), 4.95 (m, 2H), 2.03 (m, 2H), 1.25 (m, 34H), 0.52 (m, J=7.7 Hz, 2H), 0.22-−0.14 (m, 45H).

### <Compound IV-8>

Dichloromethane (1 g), the compound IV-7 (0.25 g), a toluene solution of a platinum/1,3-divinyl-1,1,3,3-tetramethyldisiloxane complex (platinum content: 3 mass %, 0.004 mg), aniline (0.3 mg), and trimethoxysilane (0.05 g) were added to a 30 mL vial, and the mixture was stirred at 25° C. for 2 hours. 0.23 g of a compound IV-8 was obtained by removing the solvent by distillation under a reduced pressure. The structure of the compound IV-8 was confirmed from NMR data shown below.

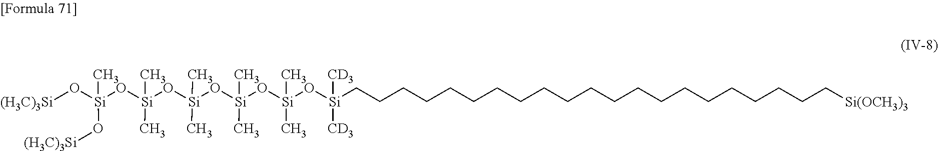

¹H-NMR (400 MHz, CDCl₃): δ=3.57 (s, 9H), 1.25 (m, 38H), 0.65 (m, 2H), 0.52 (m, 2H), 0.31-−0.14 (m, 45H).

### <Compound V-I>

The compound IV-1 (1.0 g), dichloromethane (2 mL), and triethylamine (0.17 mL) were added to a 30 mL vial, 1,7-dichloro-1,1,3,3,5,5,7,7-octamethyltetrasiloxane (1.0 g) was added dropwise, and the mixture was stirred at 25° C. for 2 hours. Hexane and ion-exchanged water were added to the reaction solution, and the organic layer was separated. 1.1 g of a compound V-1 was obtained by removing the solvent and low-boiling components by distillation under a reduced pressure and then performing flash column chromatography (developing solvent: hexane/ethyl acetate) using silica gel. The structure of the compound V-1 was confirmed from NMR data shown below.

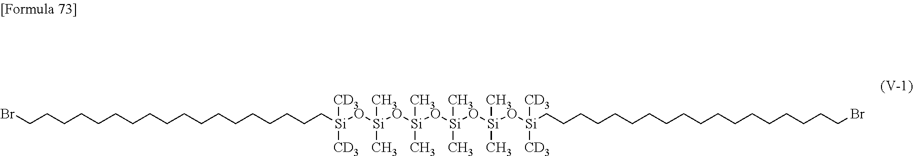 [SMILES: [2H]C([2H])([2H])[Si](CCCCCCCCCCCCCCCCCCBr)(O[Si](C)(C)O[Si](C)(C)O[Si](C)(C)O[Si](C)(C)O[Si](CCCCCCCCCCCCCCCCCCBr)(C([2H])([2H])[2H])C([2H])([2H])[2H])C([2H])([2H])[2H]]

¹H-NMR (400 MHz, CDCl₃): δ=3.41 (t, J=6.9 Hz, 4H), 1.85 (dt, J=14.5, 7.0 Hz, 4H), 1.26 (d, J=4.1 Hz, 60H), 0.52 (m, 4H), 0.29-−0.27 (m, 24H).

### <Compound V-2>

The compound V-1 (1.0 g) and a THF solution of an Allyl Grignard reagent (2.0 M, 3.0 mL) were added under a nitrogen atmosphere, and the mixture was stirred at 0° C. for 12 hours. A saturated aqueous ammonium chloride solution and hexane were successively added to the reaction solution, and the organic layer was separated. 0.7 g of a compound V-2 was obtained by removing the solvent and low-boiling components by distillation under a reduced pressure and performing flash column chromatography (developing solvent: hexane) using silica gel. The structure of the compound V-2 was confirmed from NMR data shown below.

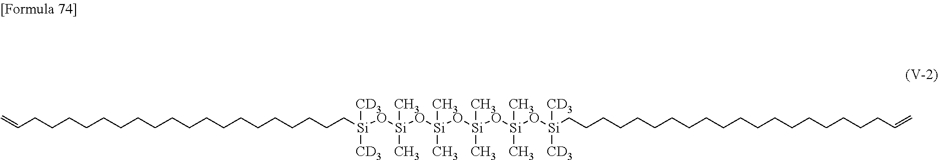 [SMILES: [2H]C([2H])([2H])[Si](CCCCCCCCCCCCCCCCCCCC=C)(O[Si](C)(C)O[Si](C)(C)O[Si](C)(C)O[Si](C)(C)O[Si](CCCCCCCCCCCCCCCCCCCC=C)(C([2H])([2H])[2H])C([2H])([2H])[2H])C([2H])([2H])[2H]]

¹H-NMR (400 MHz, CDCl₃): δ=5.81 (ddt, J=17.0, 10.2, 6.7 Hz, 2H), 4.95 (m, 4H), 2.03 (m, 4H), 1.25 (m, 68H), 0.52 (m, J=7.7 Hz, 4H), 0.22-−0.14 (m, 24H).

### <Compound V-3>

Dichloromethane (1 g), the compound V-2 (0.5 g), a toluene solution of a platinum/1,3-divinyl-1,1,3,3-tetramethyldisiloxane complex (platinum content: 3 mass %, 0.004 mg), aniline (0.3 mg), and trimethoxysilane (0.3 g) were added to a 30 mL vial, and the mixture was stirred at 25° C. for 2 hours. 0.5 g of a compound V-3 was obtained by removing the solvent by distillation under a reduced pressure. The structure of the compound V-3 was confirmed from NMR data shown below.

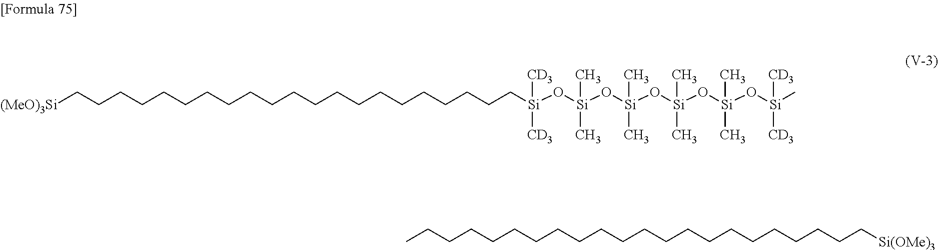

¹H-NMR (400 MHz, CDCl₃): δ=3.57 (s, 18H), 1.25 (m, 76H), 0.65 (m, 4H), 0.52 (m, 4H), 0.31-−0.14 (m, 24H).

### <Compound VI-1>

Hexane (2 mL) and methyldichlorosilane (2 g) were added to a 100 mL double-necked flask under a nitrogen atmosphere, and a hexane solution (10 mL) comprising pyridine (3.3 mL) and the compound I-3 (4 g) was added dropwise while heating this solution to 60° C. After the drop, the temperature was cooled to 25° C., and the mixture was stirred for 12 hours. The reaction solution was filtered, and hexane and ion-exchanged water were added to the filtrate, and the organic layer was separated. 2 g of a compound VI-1 was obtained by removing the solvent and low-boiling components by distillation under a reduced pressure and then performing flash column chromatography (developing solvent: hexane/ethyl acetate) using silica gel. The structure of the compound VI-1 was confirmed from NMR data shown below.

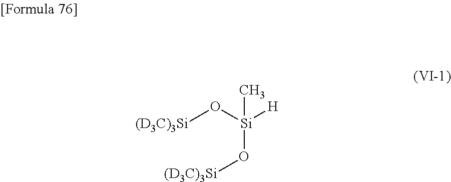

¹H-NMR (400 MHz, CDCl₃): δ=4.64 (s, 1H), 0.12 (s, 3H).

### <Compound VI-2>

18-bromo-1-octadecene (1 g), the compound VI-1 (1 g), and a toluene solution of a platinum/1,3-divinyl-1,1,3,3-tetramethyldisiloxane complex (platinum content: 3 mass %, 36 mg) were added to a 30 mL vial, and the mixture was stirred at 25° C. for 1 hour. Hexane and a saturated salt solution were added to the reaction solution, and the organic layer was separated. 1.3 g of a compound VI-2 was obtained by removing the solvent and low-boiling components by distillation under a reduced pressure and then performing flash column chromatography (developing solvent: hexane/ethyl acetate) using silica gel. The structure of the compound VI-2 was confirmed from NMR data shown below.

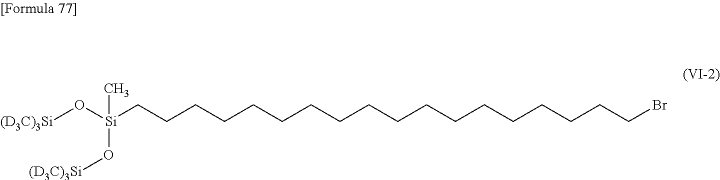

¹H-NMR (400 MHz, CDCl₃): δ=3.41 (t, J=6.9 Hz, 2H), 1.85 (m, 2H), 1.25 (s, 30H), 0.73-0.50 (m, 2H), 0.12 (s, 3H).

### <Compound VI-3>

The compound VI-2 (0.5 g) and a THF solution of an Allyl Grignard reagent (2.0 M, 1.4 mL) were added under a nitrogen atmosphere, and the mixture was stirred at 0° C. for 12 hours. a saturated aqueous ammonium chloride solution and hexane were successively added to the reaction solution, and the organic layer was separated. 0.5 g of a compound VI-3 was obtained by removing the solvent and low-boiling components by distillation under a reduced pressure and performing flash column chromatography (developing solvent: hexane) using silica gel. The structure of the compound VI-3 was confirmed from NMR data shown below.

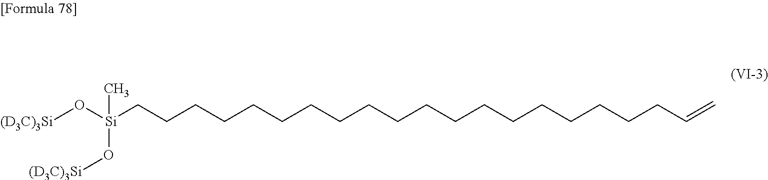

¹H-NMR (400 MHz, CDCl₃): δ=5.81 (ddt, J=17.0, 10.2, 6.7 Hz, 1H), 4.95 (m, 2H), 2.03 (m, 2H), 1.25 (m, 34H), 0.52 (m, J=7.7 Hz, 2H), 0.12 (s, 3H).

### <Compound VI-4>

Dichloromethane (1 g), the compound VI-3 (0.3 g), a toluene solution of a platinum/1,3-divinyl-1,1,3,3-tetramethyldisiloxane complex (platinum content: 3 mass %, 0.004 mg), aniline (0.3 mg), and trimethoxysilane (0.1 g) were added to a 30 mL vial, and the mixture was stirred at 25° C. for 2 hours. 0.28 g of a compound VI-4 was obtained by removing the solvent by distillation under a reduced pressure. The structure of the compound VI-4 was confirmed from NMR data shown below.

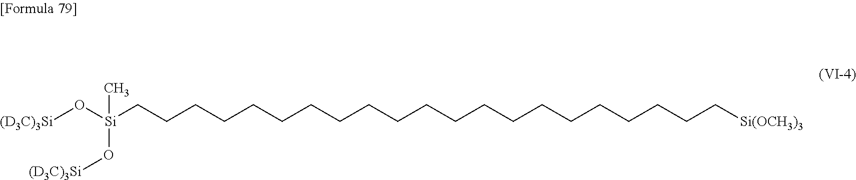

¹H-NMR (400 MHz, CDCl₃): δ=3.57 (s, 9H), 1.25 (m, 38H), 0.65 (m, 2H), 0.52 (m, 2H), 0.12 (s, 3H).

### <Compound VII-1>

17-octadecenoic acid (1 g), the compound VI-2 (1 g), and a toluene solution of a platinum/1,3-divinyl-1,1,3,3-tetramethyldisiloxane complex (platinum content: 3 mass %, 36 mg) were added to a 30 mL vial, and the mixture was stirred at 25° C. for 1 hour. Hexane and a saturated salt solution were added to the reaction solution, and the organic layer was separated. 0.4 g of a compound VII-1 was obtained by removing the solvent and low-boiling components by distillation under a reduced pressure and then performing flash column chromatography (developing solvent: hexane/ethyl acetate) using silica gel. The structure of the compound VII-1 was confirmed from NMR data shown below.

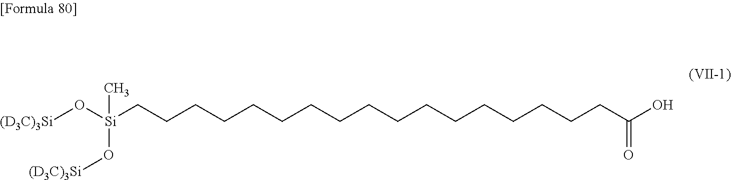

¹H-NMR (400 MHz, CDCl₃): δ=2.35 (t, J=7.6 Hz, 2H), 1.63 (m, 2H), 1.25 (s, 28H), 0.73-0.50 (m, 2H), 0.12 (s, 3H).

### <Compound VII-2>

The compound VII-1 (0.4 g), 2-allylpent-4-ene-1-amine (0.4 g), triethylamine (0.5 mL), and ethyl acetate (2 mL) were added to a 30 mL vial and the mixture was stirred. 50% Propylphosphonic Acid Anhydride, Ethyl Acetate Solution (1 mL) was added to the mixture, and the mixture was stirred at 25° C. for 12 hours. Hexane and a hydrochloric acid solution (1 M) were added to the reaction solution, and the organic layer was separated. 0.2 g of a compound VII-2 was obtained by removing the solvent and low-boiling components by distillation under a reduced pressure and then performing flash column chromatography (developing solvent: hexane/ethyl acetate) using silica gel. The structure of the compound VII-2 was confirmed from NMR data shown below.

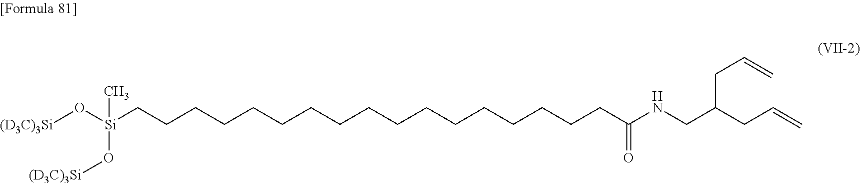

¹H-NMR (400 MHz, CDCl₃): δ=5.80 (m, 1H), 5.41 (s, 1H), 5.10 (m, 2H), 3.21 (m, 2H), 2.15 (t, J=7.6 Hz, 2H), 1.75 (m, 2H), 1.25 (s, 28H), 0.73-0.50 (m, 2H), 0.12 (s, 3H).

### <Compound VII-3>

Dichloromethane (1 g), the compound VII-2 (0.2 g), a toluene solution of a platinum/1,3-divinyl-1,1,3,3-tetramethyldisiloxane complex (platinum content: 3 mass %, 0.004 mg), aniline (0.3 mg), and trimethoxysilane (0.2 g) were added to a 30 mL vial, and the mixture was stirred at 25° C. for 2 hours. 0.22 g of a compound VII-3 was obtained by removing the solvent by distillation under a reduced pressure. The structure of the compound VII-3 was confirmed from NMR data shown below.

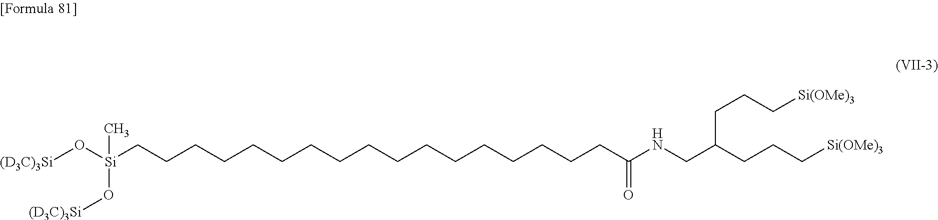

¹H-NMR (400 MHz, CDCl₃): δ=5.41 (s, 1H), 3.57 (s, 18H), 3.21 (m, 2H), 2.15 (t, J=7.6 Hz, 2H), 1.25 (m, 38H), 0.65 (m, 2H), 0.52 (m, 2H), 0.12 (s, 3H).

### <Compound VIII-1>

Triethylamine (1.5 g), dichloromethane (10 g), and chlorotrimethyl-d9-silane (0.3 g) were added to a compound IV-1 (0.5 g), and the mixture was stirred at 30° C. for 3 hours. 0.5 g of a compound VIII-1 was obtained by removing low-boiling components by distillation under a reduced pressure and performing flash column chromatography (developing solvent: hexane/dichloromethane) using silica gel. The structure of the compound VIII-1 was confirmed from NMR data shown below.

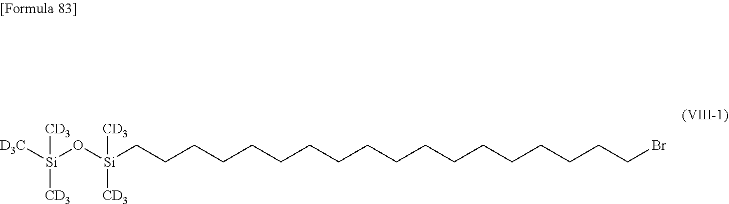 [SMILES: [2H]C([2H])([2H])[Si](CCCCCCCCCCCCCCCCCCBr)(O[Si](C([2H])([2H])[2H])(C([2H])([2H])[2H])C([2H])([2H])[2H])C([2H])([2H])[2H]]

¹H NMR (400 MHz, CDCl3) δ 3.41 (t, J=6.9 Hz, 2H), 1.99-1.72 (m, 2H), 1.49-0.97 (m, 30H), 0.50 (dd, J=9.5, 5.7 Hz, 2H).

### <Compound VIII-2>

THF (20 g) and magnesium (1.1 g) were added to 11-bromo-1-undecene (3.0 g), and the mixture was stirred at 60° C. for 2 hours. After the reaction solution was filtered, VIII-1 (0.5 g) and copper chloride (II) (0.05 g) were added, and the mixture was stirred at 40° C. for 24 hours. Hydrochloric acid and hexane were added to the reaction solution to perform extraction, low-boiling components were removed by distillation under a reduced pressure, and then flash column chromatography (developing solvent: hexane/dichloromethane) using 10% silver nitrate silica gel was performed to obtain 0.3 g of VIII-2. The structure of the compound VIII-2 was confirmed from NMR data shown below.

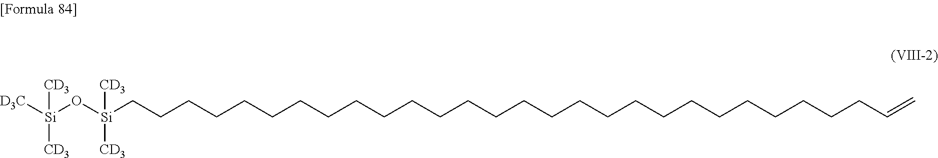 [SMILES: [2H]C([2H])([2H])[Si](CCCCCCCCCCCCCCCCCCCCCCCCCCCC=C)(O[Si](C([2H])([2H])[2H])(C([2H])([2H])[2H])C([2H])([2H])[2H])C([2H])([2H])[2H]]

¹H NMR (400 MHz, CDCl3) δ 5.81 (ddt, J=16.9, 10.1, 6.7 Hz, 1H), 5.10-4.69 (m, 2H), 2.04 (q, J=7.0 Hz, 2H), 1.49-0.97 (m, 50H), 0.49 (t, J=7.6 Hz, 2H).

### <Compound VIII-3>

A toluene solution of a platinum/1,3-divinyl-1,1,3,3-tetramethyldisiloxane complex (platinum content: 3 mass %, 5 mg), aniline (3 mg), and trimethoxysilane (0.60 g) were added to the compound VIII-2 (0.3 g) dissolved in dichloromethane (10 g), and the mixture was stirred at 50° C. for 2 hours. 0.4 g of a compound VIII-3 was obtained by removing the solvent by distillation under a reduced pressure. The structure of the compound VIII-3 was confirmed from NMR data shown below.

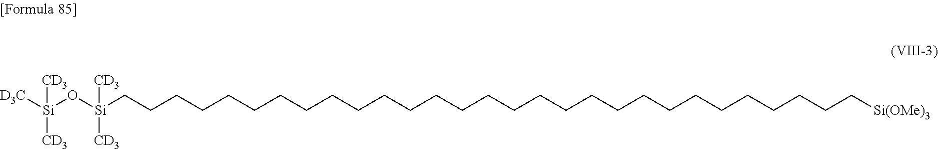

¹H NMR (400 MHz, CDCl3) δ 3.57 (s, 9H), 1.49-0.97 (m, 54H), 0.73-0.57 (m, 2H), 0.49 (t, J=6.8 Hz, 2H)

### <Compound IX-1>

Dichloromethane (10 g), trichlorosilane (3.0 g), triacetoxymethylsilane (0.05 g), and a toluene solution of a platinum/1,3-divinyl-1,1,3,3-tetramethyldisiloxane complex (platinum content: 3 mass %, 4.1 mg) were added to 18-bromo-1-octadecene (1.0 g), and the mixture was stirred at 25° C. for 2 hours. After low-boiling components were removed by distillation under a reduced pressure, a diethyl ether solution (1.0 M) (20 mL) of methyl-d3-magnesium iodide was added, and the mixture was stirred at 25° C. for 24 hours. Hydrochloric acid and hexane were added to the reaction solution to perform extraction, low-boiling components were removed by distillation under a reduced pressure, then flash column chromatography (developing solvent: hexane/dichloromethane) using silica gel was performed to obtain 1.0 g of a compound IX-1. The structure of the compound IX-1 was confirmed from NMR data shown below.

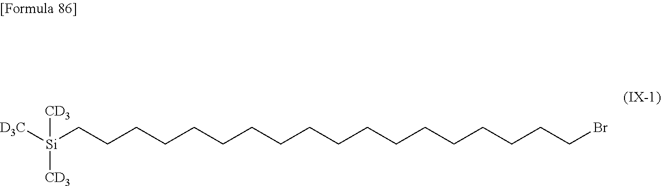 [SMILES: [2H]C([2H])([2H])[Si](CCCCCCCCCCCCCCCCCCBr)(C([2H])([2H])[2H])C([2H])([2H])[2H]]

¹H NMR (400 MHz, CDCl3) δ 3.41 (t, J=6.9 Hz, 2H), 1.90-1.69 (m, 2H), 1.47-1.00 (m, 30H), 0.48 (dq, J=10.3, 6.4, 5.7 Hz, 2H).

### <Compound IX-2>

THF (20 mL), a THF solution of allylmagnesium chloride (2.0 M, 20 mL), and copper chloride (II) (0.02 g) were added to the compound IX-1 (1.0 g), and the mixture was stirred at 50° C. for 2 hours. Hydrochloric acid and hexane were added to the reaction solution to perform extraction, low-boiling components were removed by distillation under a reduced pressure, then flash column chromatography (developing solvent: hexane/dichloromethane) using silica gel was performed to obtain 0.8 g of a compound IX-2. The structure of the compound IX-2 was confirmed from NMR data shown below.

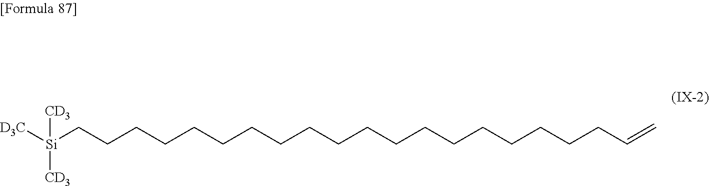 [SMILES: [2H]C([2H])([2H])[Si](CCCCCCCCCCCCCCCCCCCC=C)(C([2H])([2H])[2H])C([2H])([2H])[2H]]

¹H NMR (400 MHz, CDCl3) δ 5.84 (ddt, J=16.9, 10.2, 6.6 Hz, 1H), 5.21-4.79 (m, 2H), 2.18-1.87 (m, 2H), 1.47-1.00 (m, 34H), 0.64-0.35 (m, 2H).

### <Compound IX-3>

Dichloromethane (10 g) was added to the compound IX-2 (0.8 g) and the mixture was dissolved. Then, a toluene solution of a platinum/1,3-divinyl-1,1,3,3-tetramethyldisiloxane complex (platinum content: 3 mass %, 4.1 mg), aniline (2.6 mg), and trimethoxysilane (2.0 g) were added, and the mixture was stirred at 30° C. for 24 hours. 1.1 g of a compound IX-3 was obtained by removing the solvent by distillation under a reduced pressure. The structure of the compound IX-3 was confirmed from NMR data shown below.

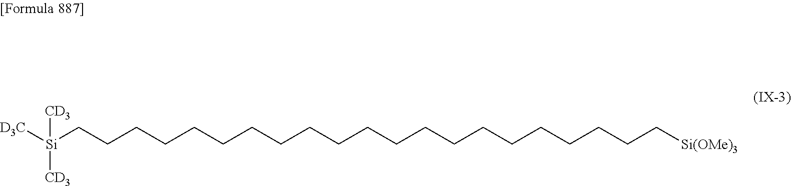

¹H NMR (400 MHz, CDCl3) δ 3.52 (s, 9H), 1.47-1.00 (m, 38H), 0.66-0.55 (m, 2H), 0.42 (t, J=7.7 Hz, 2H).

### [Manufacturing of Article]

A surface of a substrate was treated by using the compound I-5, the compound II-3, the compound III-1, the compound IV-8, the compound V-3, the compound VI-4, the compound VII-3, the compound VIII-3, and the compound IX-3 to obtain articles in Examples 1 to 3. As the surface treatment method, the following wet coating method was used for each of the examples. As the substrate, a chemically strengthened glass was used as the substrate. The obtained article was evaluated by using the following method. Table 1 shows the results.

30 g of silicon oxide was disposed as a vapor deposition source on a copper hearth in a vacuum vapor deposition apparatus (VTR-350M manufactured by ULVAC Kiko Co., Ltd.). A glass substrate was disposed in the vacuum vapor deposition apparatus, and the inside of the vacuum vapor deposition apparatus was evacuated until the pressure reached 5×10⁻³ Pa or less. The hearth was heated to about 2,000° C., and silicon oxide was vacuum-deposited on the surface of the substrate, whereby a substrate with the silicon oxide layer including a silicon oxide layer having a thickness of about 20 nm was prepared.

The substrate with the silicon oxide layer was placed on a sample stage of a spray coater (API-90RS manufactured by Apiros Co., Ltd.) so that the silicon oxide layer was on the surface. Next, 13 g of a heptane solution comprising 0.2% by mass of each compound obtained in each example was charged into a syringe in the spray coater, and spray-applied at an atomization pressure of 130 kPa, a distance between a nozzle and a sample surface of 50 mm, and a scanning speed of 300 mm/sec (wet coating method). Thereafter, the substrate with the silicon oxide layer having the surface on which the compound was applied was heat-treated at 140° C. for 30 minutes, whereby an evaluation sample (article) in which the substrate, the silicon oxide layer, and the surface treatment layer are layered in this order was obtained.

### [Evaluations]

### <Water Repellency>

Approximately 2 μL of distilled water was dropped on the surface treatment layer of the article, and the initial water contact angle was measured using a contact angle measuring apparatus (product name “DM-500”, manufactured by Kyowa Interface Science Co., Ltd.). The average value obtained by performing measurement at five locations on the surface treatment layer was set as a water contact angle. The water repellency of the surface treatment layer was evaluated based on the following criteria. The 20 method was used to calculate the water contact angle.
A: The initial water contact angle was over 1040
B: The initial water contact angle was from 100 to 1040
C: The initial water contact angle was less than 100°

### <Oil Repellency>

Approximately 2 μL of oleic acid was dropped on the surface treatment layer of the article, and the initial oil contact angle (oleic acid contact angle) was measured using a contact angle measuring apparatus (product name “DM-500”, manufactured by Kyowa Interface Science Co., Ltd.). The average value obtained by performing measurement at five locations on the surface treatment layer was set as an oil contact angle. The oil repellency of the surface treatment layer was evaluated based on the following criteria. The 20 method was used to calculate the oil contact angle.
A: The initial oil contact angle was larger than 540
B: The initial oil contact angle was from 50 to 540
C: The initial oil contact angle was less than 500

TABLE 1

Evaluations

| | | Water | Oil |
|---|---|---|---|
| | Compound | repellency | repellency |
| Example 1 | I-5 | A | A |
| Example 2 | II-3 | A | A |
| Example 3 | IV-8 | A | A |
| Example 4 | V-3 | A | A |
| Example 5 | VI-4 | A | A |
| Example 6 | VII-3 | A | A |
| Example 7 | VIII-3 | A | A |
| Example 8 | IX-3 | A | A |
| Example 9 | III-1 | B | B |

As shown in Table 1, it has been confirmed that, with the articles in Examples 1 to 8 which use the compound I-5, the compound II-3, the compound IV-8, the compound V-3, the compound VI-4, the compound VII-3, the compound VIII-3, and the compound IX-3 having the Group 1 and heavy hydrogen as surface treatment agents, a surface treatment layer having more excellent water repellency and oil repellency can be formed compared to the article in Example 9 using a compound III-1 that does not include heavy hydrogen.

A compound of the present disclosure is useful as a surface treatment agent. Such a surface treatment agent can be used, for example, for substrates in display devices such as touch panel displays, optical members, semiconductor elements, building materials, automobile components, and nanoimprinting technologies. Further, such a surface treatment agent can be used for bodies, window glasses (front glasses, side glasses, and rear glasses), mirrors, bumpers, and the like in transportation apparatuses such as trains, automobiles, ships, and airplanes. Further, such a surface treatment agent can be used for exterior walls of buildings, tents, photovoltaic modules, sound insulating plates, and outdoor articles such as concrete; and fishing nets, sweep nets, and water tanks. Further, such a surface treatment agent can be used for kitchens, bathrooms, washstands, mirrors, and toiletry components; chandeliers and ceramics such as tiles; and artificial marble and various indoor apparatuses such as air conditioners. Further, such a surface treatment agent can be used for antifouling treatments of jigs, inner walls, pipes, and the like in factories. Further, such a surface treatment agent can be used for goggles, glasses, helmets, pachinko, fibers, umbrellas, play equipment, and soccer balls. Further, such a surface treatment agent can be used as adhesion inhibitors for various packaging materials such as packaging materials for foods, packaging materials for cosmetics, and the interior of pots. Further, such a surface treatment agent can be used for car navigation systems, mobile phones, smart phones, digital cameras, digital video cameras, PDAs, portable audio players, car audios, game apparatuses, lenses for eyeglasses, camera lenses, lens filters, sunglasses, medical apparatuses such as gastroscopes, copy machines, PCs, displays (e.g., liquid crystal displays, organic EL displays, plasma displays, and touch panel displays), touch panels, protective films, and optical components such as antireflection films.

From the disclosure thus described, it will be obvious that the embodiments of the disclosure may be varied in many ways. Such variations are not to be regarded as a departure from the spirit and scope of the disclosure, and all such modifications as would be obvious to one skilled in the art are intended for inclusion within the scope of the following claims.

What is claimed is:

1. A compound comprising heavy hydrogen and the following Group 1,
wherein a ratio of the heavy hydrogen to light hydrogen is 0.1 mol % or more
—Si(R¹)ₙL₃₋ₙ Group 1:
where
R¹ is each independently a hydrocarbon group,
L is each independently a hydrolyzable group, a group having a hydrolyzable group, or a hydroxyl group, and
n is an integer from 0 to 2.

2. The compound according to claim 1, comprising at least one of the heavy hydrogen as a structure of the following Group 2 or Group 3:
—Si(CE_{b1}H_{3-b1})ₐ₁(R²)₂₋ₐ₁—O— Group 2:
-M¹(CE_{b2}H_{3-b2})ₐ₂(R²)₂₋ₐ₂-(Q)ᵣ₁-R¹⁰ Group 3:
where
E is each independently D or T,
R² is each independently a hydrocarbon group,
R¹⁰ is a hydrocarbon group, a trialkylsilyl group, a linear organopolysiloxane group, a cyclic organopolysiloxane group, or a cage-like organopolysiloxane group, and hydrogen atoms in these groups may be heavy hydrogen,
Q is —O— or —OC(═O)—,
M¹ is Si, Sn, or Ge,
r1 is 0 or 1,
a1 is 1 or 2,
b1 is 1, 2, or 3,
a2 is 1 or 2, and
b2 is 1, 2, or 3.

3. The compound according to claim 2, wherein the structure of the Group 2 or Group 3 is represented by the following Group 2a or Group 3a.
—Si(CD₃)₂-O— Group 2a:
-M¹(CD₃)₃ Group 3a:
where M¹ is Si, Sn, or Ge.

4. A compound represented by the following Formula (1a) or the following Formula (2a): 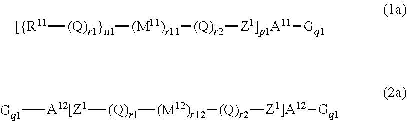
where
R¹¹ is each independently the following Group 3, or a hydrocarbon group, a trialkylsilyl group, a linear organopolysiloxane group, a cyclic organopolysiloxane group, or a cage-like organopolysiloxane group, and hydrogen atoms in these groups may be heavy hydrogen,
M¹¹ is each independently Si, Sn, Ge, or a group including a partial structure having 2 to 5 atoms selected from Si, Sn, and Ge linked together,
M¹² is each independently a group including a partial structure having 2 to 5 atoms selected from Si, Sn, and Ge linked together,
Q is each independently —O— or —OC(═O)—,
u1 is 3 when M¹¹ is Si, Sn, or Ge,
u1 is 1 when M¹¹ includes a partial structure having 2 to 5 atoms selected from Si, Sn, and Ge linked together, or when r11 is 0,
r1 is each independently 0 or 1,
r2 is each independently 0 or 1,
r11 is each independently 0 or 1,
r12 is each independently 0 or 1,
when r11 is 0, r1+r2 is 0 or 1,
when r12 is 0, r1+r2 is 0 or 1,
Z¹ is each independently an alkylene group, a polyalkylene oxide group, an organopolysiloxane group, an organopolysiloxane group including the following Group 2, a combination of an alkylene group with an organopolysiloxane group, or a combination of an alkylene group and an organopolysiloxane group including the following Group 2,
A¹¹ is a single bond or a (p1+q1)-valent linking group,
A¹² is each independently a single bond or a (1+q1)-valent linking group.
G is the following Group 1,
p1 and q1 are each independently an integer of 1 or more,
Group 1 is —Si(R¹)ₙL₃₋ₙ,
Group 2 is —Si(CE_{b1}H_{3-b1})ₐ₁(R²)₂₋ₐ₁—O—,
Group 3 is -M¹(CE_{b2}H_{3-b2})ₐ₂(R²)₂₋ₐ₂-(Q)ᵣ₁-R¹⁰,
R¹ is each independently a hydrocarbon group,
L is each independently a hydrolyzable group, a group having a hydrolyzable group, or a hydroxyl group,
n is an integer from 0 to 2,
E is each independently D or T,
R² is each independently a hydrocarbon group,
R¹⁰ is a hydrocarbon group, a trialkylsilyl group, a linear organopolysiloxane group, a cyclic organopolysiloxane group, or a cage-like organopolysiloxane group, and hydrogen atoms in these groups may be heavy hydrogen,
M¹ is Si, Sn, or Ge,
a1 is 1 or 2,
b1 is 1, 2, or 3,
a2 is 1 or 2, and
b2 is 1, 2, or 3,
where in Formula (1a), at least one of R¹¹ is Group 3, or at least one of R¹¹ and Z¹ includes an organopolysiloxane group including Group 2, and
in Formula (2a), at least one of Z¹ includes an organopolysiloxane group including Group 2.

5. A compound represented by the following Formula (1b) or the following Formula (2b): 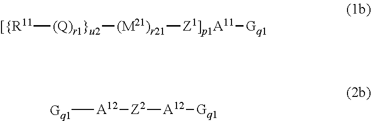
where
R¹¹ is each independently the following Group 3, or a hydrocarbon group, a trialkylsilyl group, a linear organopolysiloxane group, a cyclic organopolysiloxane group, or a cage-like organopolysiloxane group, and hydrogen atoms in these groups may be heavy hydrogen,
Q is each independently —O— or —OC(═O)—,
r1 is each independently 0 or 1,
M²¹ is Si, Sn, or Ge,
r21 is 0 or 1,
u2 is 1 when r21 is 0 and is 3 when r21 is 1,
Z¹ is each independently an alkylene group, a polyalkylene oxide group, an organopolysiloxane group, an organopolysiloxane group including the following Group 2, a combination of an alkylene group with an organopolysiloxane group, or a combination of an alkylene group and an organopolysiloxane group including the following Group 2,
Z² is an organopolysiloxane group including the following Group 2, or a combination of an alkylene group and an organopolysiloxane group including the following Group 2,
A¹¹ is a single bond or a (p1+q1)-valent linking group,
A¹² is each independently a single bond or a (1+q1)-valent linking group,
G is the following Group 1,
p1 and q1 are each independently an integer of 1 or more,
Group 1 is —Si(R¹)ₙL₃₋ₙ,
Group 2 is —Si(CE_{b1}H_{3-b1})ₐ₁(R²)₂₋ₐ₁—O—,
Group 3 is -M¹(CE_{b2}H_{3-b2})ₐ₂(R²)₂₋ₐ₂-(Q)ᵣ₁-R¹⁰,
R¹ is each independently a hydrocarbon group,
L is each independently a hydrolyzable group, a group having a hydrolyzable group, or a hydroxyl group,
n is an integer from 0 to 2,
E is each independently D or T,
R² is each independently a hydrocarbon group,
R¹⁰ is a hydrocarbon group, a trialkylsilyl group, a linear organopolysiloxane group, a cyclic organopolysiloxane group, or a cage-like organopolysiloxane group, and hydrogen atoms in these groups may be substituted with the E,
M¹ is Si, Sn, or Ge,
a1 is 1 or 2,
b1 is 1, 2, or 3,
a2 is 1 or 2, and
b2 is 1, 2, or 3,
where in Formula (1b), at least one of R¹¹ is Group 3, or at least one of R¹¹ and Z¹ includes an organopolysiloxane group including Group 2.

6. The compound according to claim 4, wherein the Z¹ is each independently a group represented by the following Formula (3): 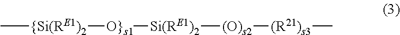
where
R^{E1} is each independently a hydrocarbon group in which a hydrogen atom may be heavy hydrogen,
R²¹ is an alkylene group,
s1 is an integer from 0 to 500,
s2 is 0 or 1, and
s3 is 0 or 1.

7. The compound according to claim 5, wherein the Z² is a group represented by the following Formula (4): 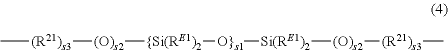
where
R^{E1} is each independently a hydrocarbon group in which a hydrogen atom may be heavy hydrogen,
R²¹ is each independently an alkylene group,
s1 is an integer from 0 to 500,
s2 is each independently 0 or 1, and
s3 is each independently 0 or 1.

8. A compound represented by the following Formula (1c) or the following Formula (2c): 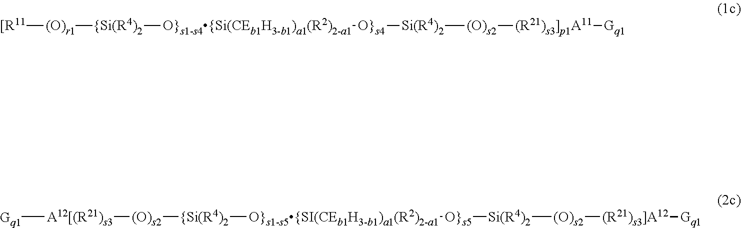
where
R¹¹ is each independently a hydrocarbon group, a trialkylsilyl group, a linear organopolysiloxane group, a cyclic organopolysiloxane group, a cage-like organopolysiloxane group, or the following Group 3,
R⁴ is each independently a hydrocarbon group,
E is each independently D or T,
R²¹ is each independently an alkylene group,
r1 is each independently 0 or 1,
a1 is 1 or 2,
b1 is 1, 2, or 3,
s1 is an integer from 0 to 500,
s2 is each independently 0 or 1,
s3 is each independently 0 or 1,
s4 is an integer from 0 to s1, and is an integer of 1 or more when R¹¹ is a group other than the Group 3,
s5 is an integer from 1 to s1,
A¹¹ is a single bond or a (p1+q1)-valent linking group,
A¹² is each independently a single bond or a (1+q1)-valent linking group.
G is the following Group 1,
p1 and q1 are each independently an integer of 1 or more,
Group 1 is —Si(R¹)ₙL₃₋ₙ,
Group 3 is -M¹(CE_{b2}H_{3-b2})ₐ₂(R²)₂₋ₐ₂-(Q)ᵣ₁-R¹⁰,
R¹ is each independently a hydrocarbon group,
L is each independently a hydrolyzable group, a group having a hydrolyzable group, or a hydroxyl group,
n is an integer from 0 to 2,
E is each independently D or T,
R² is each independently a hydrocarbon group,
R¹⁰ is a hydrocarbon group, a trialkylsilyl group, a linear organopolysiloxane group, a cyclic organopolysiloxane group, or a cage-like organopolysiloxane group, and hydrogen atoms in these groups may be substituted with the E,
M¹ is Si, Sn, or Ge,
a2 is 1 or 2, and
b2 is 1, 2, or 3.

9. The compound according to claim 8, wherein s1 is an integer of 1 or more.

10. The compound according to claim 8, wherein s3 is 1.

11. The compound according to claim 8, wherein the p1 is an integer from 1 to 4.

12. The compound according to claim 8, wherein the q1 is each independently an integer from 1 to 4.

13. A composition comprising the compound according to claim 1 and a liquid medium.

14. A surface treatment agent comprising the compound according to claim 1.

15. A surface treatment agent comprising the compound according to claim 1 and a liquid medium.

16. A method for manufacturing an article including a surface treatment layer formed on a substrate by performing a surface treatment on the substrate by using a surface treatment agent comprising the compound according to claim 1.

17. An article comprising a substrate, and a surface treatment layer disposed on the substrate, a surface of the surface treatment layer being treated with a surface treatment agent comprising the compound according to claim 1.

18. The article according to claim 17, wherein the article is an optical member.

19. The article according to claim 17, wherein the article is a display or a touch panel.
